(19) United States
(12) Patent Application Publication
(10) Pub. No.: US 2011/0319431 A1
(43) Pub. Date: Dec. 29, 2011

(54) Bis Aromatic Compounds for Use as LTC4 Synthase Inhibitors

(75) Inventors: Peter Nilsson, Uppsala (SE); Benjamin Pelcman, Uppsala (SE); Martins Katkevics, Riga (LV)

(73) Assignee: BIOLIPOX AB, Uppsala (SW)

(21) Appl. No.: 13/256,122

(22) PCT Filed: Mar. 15, 2010
(86) PCT No.: PCT/GB2010/000481

§ 371 Date: Sep. 12, 2011

Related U.S. Application Data
(60) Provisional application No. 61/202,559, filed on Mar. 12, 2009.

(30) Foreign Application Priority Data
Mar. 12, 2010 (GB) PCT/GB2010/000438
Mar. 12, 2010 (GB) PCT/GB2010/000439

(51) Int. Cl. A61K 31/505; A61K 31/196; C07D 239/47; C07D 213/78; C07C 59/84; A61P 31/12; A61P 11/00; A61P 29/00; A61P 37/08; A61P 11/06; A61P 9/00; A61P 27/16; A61P 27/02; A61P 17/00; A61P 1/00; A61P 13/00; A61P 25/00; A61P 7/06; A61P 31/04; A61P 31/10; A61K 31/44

(52) U.S. Cl. 514/256; 514/352; 514/562; 544/322; 546/310; 562/460

(57) ABSTRACT

There is provided compounds of formula I,

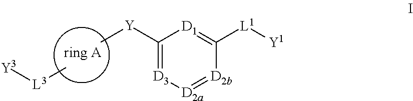

wherein Y, ring A, D₁, D₂ₐ, D_{2b}, D₃, L¹, Y¹, L³ and Y³ have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. They may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRas) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRas have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy.

Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

European patent application EP 344 775 discloses various biaryl/diaryl compounds for use as herbicides. However, there is no mention in that document of compounds containing three aromatic rings, nor does this document indicate that those compounds may be useful as medicaments.

International patent application WO 2008/107661 discloses various biphenyl/diphenyl compounds that may be useful as inhibitors of LTC₄ synthase, and therefore of use in the treatment of inflammation. However, the two phenyl rings are linked together with via a methylene group. Further, international patent application WO 2009/030887 discloses, for the same use, various biaryl compounds that may be linked via a carbonyl group (i.e. diarylketones). However, the document predominantly relates to biaryl/diaryl ring systems that are not further substituted via a sulfur containing linker group.

### DISCLOSURE OF THE INVENTION

According to the invention, there is provided a compound of formula I,

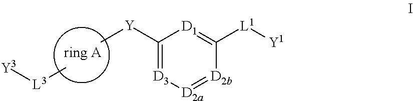

wherein
Y represents —C(O)— or —C(═N—OR²⁸)—;
either one of D₂ₐ and D_{2b} represents D₂, and the other represents —C(-L²-Y²)═;
R²⁸ represents hydrogen or C₁₋₆ alkyl optionally substituted by one or more halo atoms;
each of D₁, D₂ and D₃ respectively represent —C(R^{1a})═, —C(R^{1b})═ and —C(R^{1c})═, or,
each of D₁, D₂ and D₃ may alternatively and independently represent —N═;
ring A represents:

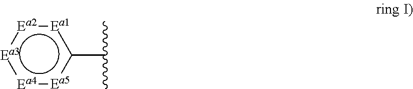

each of E^{a1}, E^{a2}, E^{a3}, E^{a4} and E^{a5} respectively represent —C(H)═, —C(R^{2b})═, —C(R^{2c})═, —C(R^{2d})═ and —C(H)═, or, each of E^{a1}, E^{a2}, E^{a3}, E^{a4} and E^{a5} may alternatively and independently represent —N═;
one of R^{2b}, R^{2c} and R^{2d} represents the requisite -L³-Y³ group, and the others independently represent hydrogen, -L^{1a}-Y^{1a} or a substituent selected from X¹;

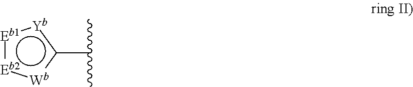

E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or

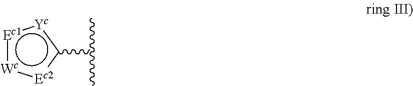

E^{c1} and E^{c2} each respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c} and R^{4d} represents the requisite -L³-Y³ group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b}, R^{1c}, independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{2a} independently represent —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}R⁵ⁿ, R^{6a} to R⁶ⁱ, R^{7a}, R^{7b}, R^{7d} and R^{7f} to R⁷ⁱ independently represent, on each occasion when used herein, H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O, —OR^{5h} and/or R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and/or —OS(O)₂N(R^{8g})R^{8h};
n represents 0, 1 or 2;
R^{8a}, R^{8b}, R^{8d}, R^{8a} and R^{8g} independently represent H or C₁₋₆ alkyl optionally substituted by one or more substituents selected from halo, ═O, —OR^{11a}, —N(R^{12a})R^{12b} and/or —S(O)₂-M¹;
R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and/or —S(O)₁-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O and/or C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, R^{15a} and R^{15b} independently represent H, —CH₃ or —CH₂CH₃,
Y¹ and Y^{1a} independently represent, on each occasion when used herein, —C(O)OR^{9a} or 5-tetrazolyl;
R^{9a} represents:
(i) hydrogen; or
(ii) C₁₋₈ alkyl or a heterocycloalkyl group, both of which are optionally substituted by one or more substituents selected from G¹ and/or Z¹;
one of Y² and Y³ represents an aryl group or a heteroaryl group (both of which groups are optionally substituted by one or more substituents selected from A) and the other represents either:
(a) an aryl group or a heteroaryl group (both of which groups are optionally substituted by one or more substituents selected from A); or
(b) C₁₋₁₂ alkyl or a heterocycloalkyl group, both of which are optionally substituted by one or more substituents selected from G¹ and/or Z¹;
A represents, on each occasion when used herein:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or −A¹—R^{16a};
wherein A¹ represents a single bond or a spacer group selected from —C(O)A²-, —S—, —S(O)ₘ₁A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents, on each occasion when used herein, ═O, ═S, ═NOR^{16b}, ═NS(O)₂N(R^{17f})R^{16c}, ═NCN or ═C(H)NO₂;
B represents, on each occasion when used herein:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —S(O)ₘ₁A⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁹ independently represent a single bond, —C(O)—, —C(O)N(R^{19d})—, —C(O)O—, —S(O)₂— or —S(O)₂N(R^{19e})—;
Z² represents, on each occasion when used herein, ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R¹⁹c, R^{19d}, R^{18e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{18a} to R^{18f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹—R^{20a};
wherein A¹¹ represents a single bond or a spacer group selected from —C(O)A¹²-, —S—, —S(O)ₘ₁A^{b 13}-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents, on each occasion when used herein, ═O, ═S, ═NOR^{20b}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L¹ and L^{1a} independently represent a single bond or —(CH₂)ₚ-Q-(CH₂)_{q}—;
Q represents —C(R^{y1})(R^{y2})—, —C(O)— or —O—;
R^{y1} and R^{y2} independently represent H, F or X⁴; or
R^{y1} and R^{y2} may be linked together to form a 3- to 6-membered ring, which ring optionally contains a heteroatom, and which ring is optionally substituted by one or more substituents selected from F, Cl, ═O and X⁵;
L² represents —S(O)ᵣ-A²¹-;
L³ represents a single bond or a spacer group selected from —(CH₂)ₚ—C(R^{y3})(R^{y4})—(CH₂)_{q}-A¹⁶-, —C(O)A¹⁷-, —S—, —S(O)—, —SC(R^{y3})(R^{y4})—, —S(O)₂A¹⁸-, —N(R^{w})A¹⁹- or —OA²⁰-, in which:
A¹⁶ represents a single bond, —O—, —N(R^{w})—, —C(O)—, or —S(O)ₘ—;
A¹⁷ and A¹⁸ independently represent a single bond, —C(R^{y3})(R^{y4})—, —O—, or —N(R^{w});
A¹⁹ and A²⁰ independently represent a single bond, —C(R^{y3})(R^{y4})—, —C(O)—, —C(O)C(R^{y3})(R^{y4})—, —C(O)N(R^{w})—, —C(O)O—, —S(O)₂— or —S(O)₂N(R^{w})—;
A²¹ A represents a single bond or —C(R^{y3})(R^{y4})—;
p, q and r independently represent, on each occasion when used herein, 0, 1 or 2;
m represents, on each occasion when used herein, 0, 1 or 2;
m1 represents, on each occasion when used herein, 1 or 2;
R^{y3} and R^{y4} independently represent, on each occasion when used herein, H, F or X⁶; or
R^{y3} and R^{y4} may be linked together to form a 3- to 6-membered ring, which ring optionally contains a heteroatom, and which ring is optionally substituted by one or more substituents selected from F, Cl, ═O and X⁷;
R^{w} represents, on each occasion when used herein, H or X⁸;
X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, —CN, C₁₋₄ alkyl (optionally substituted by one or more substituents selected from fluoro, chloro and ═O), —N(R^{24c})R^{25b} and —OR^{24d})), aryl or heteroaryl (which latter two groups are optionally substituted by one or more substituents selected from halo, —CN, C₁₋₄ alkyl (optionally substituted by one or more substituents selected from fluoro, chloro and ═O), —N(R^{26a})R^{26b} and —OR^{26c});
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b} and R^{26c} are independently selected from hydrogen and C₁₋₄ alkyl, which latter group is optionally substituted by one or more substituents selected from fluoro, —OH, —OCH₃, —OCH₂CH₃ and/or ═O,
or a pharmaceutically-acceptable salt thereof,
which compounds and salts are referred to hereinafter as “the compounds of the invention”. Such compounds of the invention are characterised in that L² represents —S(O)ᵣ—.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group). Where the number of carbon atoms permits, C_{1-q} alkyl groups may also be spiro-groups (i.e. two cycloalkyl rings linked together by a single common carbon atom), although they are preferably not so.

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} (e.g. C_{4-q}) heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl) and, more preferably, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselena-diazolyl (including 2,1,3-benzoselenadiazolyl), benzothienyl, carbazolyl, chromanyl, cinnolinyl, furanyl (i.e. furyl), imidazolyl, imidazopyridyl (such as imidazo[4,5-b]pyridyl, imidazo[5,4-b]pyridyl and, preferably, imidazo[1,2-a]pyridyl), indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are polycyclic, they are preferably linked to the rest of the molecule via an aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which X¹ and X² both represent R^{5a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when there are two X¹ substituents present, which represent —R^{5a} and —C(O)R^{5b} in which R^{5b} represents R^{5a}, then the identities of the two R^{3a} groups are not to be regarded as being interdependent. Likewise, when Y² or Y³ represent e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{5a} to R^{5h}” is employed herein, this will be understood by the skilled person to mean R^{5a}, R^{5b}, R^{5c}, R^{5d}, R^{5e}, R^{5f}, R^{5g} and R^{5h} inclusively. For the avoidance of doubt, when the term “an R⁵ group” is referred to herein, we mean any one of R^{5a} to R^{5k}, R^{5m}, R⁵ⁿ or R^{5p}.

For the avoidance of doubt, where it is stated herein that “any pair of R^{16a} to R^{16c} and R^{17a} to R¹⁷ . . . may . . . be linked together”, we mean that any one of R^{16a}, R^{16b} or R^{16c} may be linked with any one of R^{17a}, R^{17b}, R^{17c}, R^{17d}, R^{17e} or R^{17f} to form a ring as hereinbefore defined. For example, R^{16a} and R^{17b} (i.e. when a G¹ group is present in which G¹ represents -A¹-R^{16a}, A¹ represents —C(O)A² and A² represents —N(R^{17b})—) or R^{16c} and R^{17f} may be linked together with the nitrogen atom to which they are necessarily attached to form a ring as hereinbefore defined.

The skilled person will appreciate that, given that there is an essential ‘-L³-Y³’ group present in the compound of formula I, then when, for example, ring A represents ring I), then at least one of —C(R^{2b})═, —C(R^{2c})═ and —C(R^{2d})═ must be present, in which the any one of the relevant R^{2b}, R^{2c} and R^{2d} groups represents the essential -L³-Y³ group.

Compounds of the invention that may be mentioned include those in which A²¹ represents a single bond, i.e. L² represents —S(O)ᵣ—.

Compounds of the invention that may be mentioned include those in which:

the D₁ to D₃-containing ring does not contain —N═ (i.e. D₁, D₂ and D₃ respectively represent —C(R^{1a})═, —C(R^{1b})═ and —C(R^{1c})═);
ring A (e.g. when it represents ring (I)) does not contain —N═ (e.g. E^{a1}, E^{a2}, E^{a3}, E^{a4} and E^{a5} respectively represent —C(H)═, —C(R^{2b})═, —C(R^{2c})═, —C(R^{2d})═ and —C(H)═); D_{2b} represents —C(-L²-Y²)═;
Z^{1a} and Z^{2a} do not represent —C(O)OR^{5c} (i.e. each independently represent —R^{5a}, —C(O)R^{5b}, —C(O)N(R^{6a})R^{7a}, —S(O)ₘR^{5j} or —S(O)₂N(R^{6h})R^{7h}).

Further compounds of the invention that may be mentioned include those in which:

D_{2b} represents —C(-L²-Y²)═;
D₂ represents —C(R^{1b})═, in which R^{1b} represents halo, —CN or, preferably, hydrogen;
D₁, D₂ and D₃ respectively represent —C(R^{1a})═, —C(R^{1b})═ and —C(R^{1c})═, in which R^{1a}, R^{1b} and R^{1c} independently represent hydrogen, halo or —CN (preferably, hydrogen).

Compounds of the invention that may be mentioned include those in which, for example, when D₁, D₂ and D₃ respectively represent —C(R^{1a})═, —C(R^{1b})═ and —C(R^{1c})═; ring A represents ring (I) and E^{a1}, E^{a2}, E^{a3}, E^{a4} and E^{a5} respectively represent —C(H)═, —C(R^{2b})═, —C(R^{2c})═, —C(R^{2d})═ and —C(H)═, then:

when Y² and Y³ both represent a heteroaryl (e.g. a 4- to 10-membered heteroaryl) group, then L¹ and, if present, L^{1a}, independently represent a single bond, —(CH₂)ₚ-Q-(CH₂)_{q}— in which Q represents —C(O)—, or, —(CH₂)ₚ-Q-(CH₂)_{q}— in which p represents 1 or 2 and Q represents —O—;
when R^{5a} represents C₁₋₆ alkyl substituted with two substituents, then those substituents are not ═O and —OR^{8a} substituted at a terminal carbon atom of the alkyl group (so forming a —C(═O)OR^{8a} group);
when R^{5a} represents C₁₋₆ alkyl substituted with two substituents, then those substituents are not ═O and —N(R^{8b})R^{8c} substituted at a terminal carbon atom of the alkyl group (so forming a —C(═O)N(R^{8b})R^{8c} group);
when R^{8a}, R^{8b}, R^{8d}, R^{8e} and/or R^{8g} represent C₁₋₆ alkyl substituted with two substituents, then those substituents are not ═O and —OR^{11a} substituted at a terminal carbon atom of the alkyl group (so forming a —C(═O)OR^{11a} group);
when R^{8a}, R^{8b}, R^{8d}, R^{8e} and/or R^{89g} represent C₁₋₆ alkyl substituted with two substituents, then those substituents are not ═O and —N(R^{12a})R^{12b} substituted at a terminal carbon atom of the alkyl group (so forming a —C(═O)N(R^{12a})R^{12b} group);
when R^{8c}, R^{8f} and/or R^{8h} represent C₁₋₆ alkyl substituted with two substituents, then those substituents are not ═O and —OR^{13a} substituted at a terminal carbon atom of the alkyl group (so forming a —C(═O)OR^{13a} group);
when R^{8c}, R^{8f} and/or R^{8h} represent C₁₋₆ alkyl substituted with two substituents, then those substituents are not ═O and —N(R^{14a})R^{14b} substituted at a terminal carbon atom of the alkyl group (so forming a —C(═O)N(R^{14a})R^{14b} group);
when Y² and Y³ both represent a heteroaryl group, then L² and L³ do not both represent single bonds.

Further compounds of the invention that may be mentioned include those in which, for example, when D₁, D₂ and D₃ respectively represent —C(R^{1a})═, —C(R^{1b})═ and —C(R^{1c})═; ring A represents ring (I) and E^{a1}, E^{a2}, E^{a3}, E^{a4} and E^{a5} respectively represent —C(H)═, —C(R^{2b})═, —C(R^{2c})═, —C(R^{2d})═ and —C(H)═, then:

L¹ represents a single bond, —(CH₂)ₚ-Q-(CH₂)_{q}— in which Q represents —C(O)—, or, —(CH₂)ₚ-Q-(CH₂)_{q}— in which p represents 1 or 2 and Q represents —O—;
Q represents —C(O)—;
R^{5a} represents, on each occasion when used herein, C₁₋₆ alkyl optionally substituted by one or more substituents selected from halo, —CN, —N₃, —OR^{8a}, —N(R^{8b})R^{8c}, —S(O)ₙR^{8d}, —S(O)₂N(R^{8e})R^{8f} or —OS(O)₂N(R^{8g})R^{8h};
R^{5a} represents, on each occasion when used herein, C₁₋₆ alkyl optionally substituted by one or more substituents selected from halo, —CN, —N₃, ═O, —N(R^{8b})R^{8c}, —S(O)ₙR^{8d}, —S(O)₂N(R^{8e})R^{8f} or —OS(O)₂N(R^{8g})R^{8h};
one of L² and L³ represents —S(O)ᵣ— and the other independently represents a spacer group selected from —(CH₂)ₚ—C(R^{y3})(R^{y4})—(CH₂)_{q}-A¹⁶-, —C(O)A¹⁷-, —S—, —S(O)—, —SC(R^{y3})(R^{y4})—, —S(O)₂A¹⁸-, —N(R^{w})A¹⁹ or —OA²⁰-;
(e.g. one of) Y² and Y³ represent an aryl group optionally substituted as defined herein.

Further compounds of the invention that may be mentioned include those in which, for example, when D₁, D₂ and D₃ respectively represent —C(H)═, —C(R^{1b})═ and —C(H)═; ring A represents ring (I) and E^{a1}, E^{a2}, E^{a3}, E^{a4} and E^{a5} respectively represent —C(H)═, —C(R^{2b})═, —C(R^{2c})═, —C(R^{2d})═ and —C(H)═, when R^{1b} or, if present, X¹ represent —N(R^{5d})C(O)R^{6c}, and R^{6c} represents R^{5a}, then R^{5a} represents a linear or branched C₁₋₆ alkyl group optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} or —OS(O)₂N(R^{8g})R^{8h}.

Yet further compounds of the invention that may be mentioned include those in which:

when, for example, ring A represents ring (I), L² or L³ represent —N(R^{w})A¹⁶-, in which A¹⁹ represents a single bond and R^{w} represents H, then Y² or Y³ (as appropriate) do not represent a benzimidazolyl (e.g. benzimidazol-2-yl) group. Preferred compounds of the invention that may be mentioned include those in which:
M¹ and M² independently represent —CH₂CH₃, or, preferably, —CH₃, —CF₃ or —N(R^{15a})R^{15b};
R^{11a} and R^{13a} independently represent —CHF₂ or, preferably H, —CH₃, —CH₂CH₃ or —CF₃;
X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d})), aryl or heteroaryl (which latter two groups are optionally substituted by one or more substituents selected from halo, C₁₋₄ alkyl (optionally substituted by one or more substituents selected from fluoro, chloro and ═O), —N(R^{26a})R^{26b} and —OR²⁶c);
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b} and R^{26c} are independently selected from hydrogen and C₁₋₄ alkyl, which latter group is optionally substituted by one or more substituents selected from chloro or, preferably, fluoro and/or ═O;
when an alkyl group is substituted with halo, then that halo atom is preferably fluoro.

Preferred compounds of the invention include those in which:

D₂ₐ represents D₂;
D_{2b} represents —C(-L²-Y²)═;
one (e.g. D₁ or D₃) or none of D₁, D₂ and D₃ represent —N═;
D₁, D₂ and D₃ respectively represent —C(R^{1a})═, —C(R^{1b})═ and —C(R^{1c})═;
R^{1a} and R^{1c} independently represent hydrogen;
when ring A represents ring (I), then two, preferably, one or, more preferably, none of E^{a1}, E^{a2}, E^{a3}, E^{a4} and E^{a5} represent —N═;
E^{a1}, E^{a2}, E^{a3}, E^{a4} and E^{a5} respectively represent —C(H)═, —C(R^{2b})═, —C(R^{2c})═, —C(R^{2d})═ and —C(H)═;
R^{2c} represents the requisite -L³-Y³ group;
only one, of R^{2b}, R^{2c} and R^{2d} (e.g. R^{2b}) may represent -L^{1a}-Y^{1a};
one of R^{2b} and R^{2d} (e.g. R^{2b}) represents hydrogen or -L^{1a}-Y^{1a}, and the other represents hydrogen or a substituent selected from X¹;
when one of R^{2b}, R^{2c} and R^{2d} represents -L^{1a}-Y^{1a}, then it is preferably tetrazolyl or, more preferably, —COOR^{9a}, in which R^{9a} is preferably H;
R^{3c} and R^{3d} independently represent unsubstituted C₁₋₆ (e.g. C₁₋₃) alkyl, or, preferably, hydrogen;
for example when ring A represents ring (II) then, one of R^{3a} and R^{3b} represents a substituent X² or, more preferably, H or -L^{1a}-Y^{1a}, and the other represents the requisite -L³-Y³ group;
R^{4b} and R^{4c} independently represent unsubstituted C₁₋₆ (e.g. C₁₋₃) alkyl, or, preferably, hydrogen;
for example when ring A represents ring (III) then, one of R^{4a} and, if present, R^{4d} represents a substituent X³ or, more preferably, H or -L^{1a}-Y^{1a}, and the other represents the requisite -L³-Y³ group;
when any one of R^{3a}, R^{3b}, R^{3c}, R^{3d}, R^{4a}, R^{4b}, R^{4c} or R^{4d} (e.g. R^{3a}, R^{3b}, R^{4a} or R^{4d}) represents -L^{1a}-Y^{1a}, then it is preferably a 5-tetrazolyl group or —COOR^{9a}, in which R^{9a} is preferably H;
X¹, X² and X³ independently represent halo (e.g. chloro or fluoro), —R^{5a}, —CN, and —OR^{5h};
Z^{1a} and Z^{2a} independently represent —R^{5a};
when any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ are linked together, they form a 5- or 6-membered ring optionally substituted by F, —OCH₃ or, preferably, ═O or R^{5a}, and which ring optionally contains an oxygen or nitrogen heteroatom (which nitrogen heteroatom may be optionally substituted, for example with a methyl group, so forming e.g. —N(H)— or —N(CH₃)—);
R^{5c}, R^{5j} and R^{6e} independently represent R^{5a};
when R^{5a}, R^{8a}, R^{8b}, R^{8d}, R^{8e} and R^{8g} represent C₁₋₆ alkyl optionally substituted by one or more halo substituents, then those halo substituents are preferably Cl or, more preferably, F;
R^{5a} represents C₁₋₆ (e.g. C₁₋₄) alkyl optionally substituted by one or more substituents selected from Cl, ═O, —N(R^{8b})R^{8c} and, preferably, F and —OR^{8a}; m and n independently represent 2;
when any one of R^{8a}, R^{8b}, R^{8d}, R^{8e} and R^{8g} represents C₁₋₆ alkyl substituted by halo, then preferred halo groups are chloro and, preferably, fluoro;
R^{8a}, R^{8b}, R^{8d}, R^{8e} and R^{8g} independently represent H or C₁₋₃ alkyl optionally substituted by one or more fluoro atoms;
R^{8c}, R^{8f} and R^{8h} independently represent H, —S(O)₂CH₃, —S(O)₂CF₃ or C₁₋₃ alkyl optionally substituted by one or more fluoro atoms, or the relevant pairs (i.e. R^{8b} and R^{8c}, R^{8e} and R^{8f} or R^{8g} and R^{8h}) are linked together as defined herein;
when R^{8b} and R^{8c}, R^{8e} and R^{8f} or R^{8g} and R^{8h} are linked together, they form a 5- or 6-membered ring, optionally substituted by F, ═O or —CH₃;
M¹ and M² independently represent —CH₃ or —CF₃;
R^{11a}, R^{12a}, R^{12b}, R^{13a}, R^{14a}, R^{14b}, R^{15a} and R^{15b} independently represent H or —CH₃;
R^{9a} represents C₁₋₄ (e.g. C₁₋₃) alkyl optionally substituted by one or more halo (e.g. fluoro) atoms, or, R^{9a} more preferably represents hydrogen;
A represents aryl (e.g. phenyl) optionally substituted by B; C₁₋₆ alkyl optionally substituted by G¹ and/or Z¹; or G¹;
G¹ represents halo, cyano or -A¹-R^{16a};
A¹ represents —C(O)A², —N(R^{17a})A⁴- or —OA⁵-;
A² represents a single bond or —O—;
A⁴ represents —C(O)N(R^{17d})—, —C(O)O— or, more preferably, a single bond or —C(O)—;
A⁵ represents —C(O)— or, preferably, a single bond;
Z¹ represents ═NCN, preferably, ═NOR^{16b} or, more preferably, ═O;
B represents heteroaryl (e.g. oxazolyl, thiazolyl, pyridyl or, preferably, thienyl) or, more preferably, aryl (e.g. phenyl) optionally substituted by G²; C₁₋₆ alkyl optionally substituted by G² and/or Z²; or, preferably G²,
G² represents cyano or, more preferably, halo or -A⁶-R^{18a};
A⁶ represents a single bond, —N(R^{19a})A⁹- or —OA¹⁰-;
A⁹ represents —C(O)N(R^{19d})—, —C(O)O— or, more preferably, a single bond or —C(O)—;
A¹⁰ represents a single bond;
Z² represents ═NCN, preferably, ═NOR^{18b} or, more preferably, ═O;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{18f} are independently selected from hydrogen, aryl (e.g. phenyl) or heteroaryl (which latter two groups are optionally substituted by G³) or C₁₋₆ (e.g. C₁₋₄) alkyl (optionally substituted by G³ and/or Z³), or the relevant pairs are linked together as hereinbefore defined;
when any pair of R^{16a} to R^{16c} and R^{17a} to R^{17f}, or R^{18a} to R^{18c} and R^{19a} to R^{19f} are linked together, they form a 5- or 6-membered ring, optionally substituted by one or more (e.g. one or two) substituents selected from G³ and/or Z³;
G³ represents halo or -A¹¹-R^{20a};
A¹¹ represents a single bond or —O—;
Z³ represents ═O;
R^{20a}, R^{20b}, R^{20c}, R^{21a}, R^{21b}; R^{21c}, R^{21d}, R^{21e} and R^{21f} are independently selected from H, C₁₋₃ (e.g. C₁₋₂) alkyl (e.g. methyl) optionally substituted by one or more halo (e.g. fluoro) atoms, or optionally substituted aryl (e.g. phenyl), or the relevant pairs are linked together as defined herein;
when any pair of R^{20a} to R^{20c} and R^{21a} to R^{21f} are linked together, they form a 5- or 6-membered ring, optionally substituted by one or more (e.g. one or two) substituents selected from halo (e.g. fluoro) and C₁₋₂ alkyl (e.g. methyl);
R^{y1} and R^{y2} independently represent hydrogen or methyl, or, they are linked together to form a 3-membered cyclopropyl group;
either one of p and q represents 1 and the other represents 0, or, more preferably, both of p and q represent 0;
Q represents —C(R^{y1})(R^{y2})— or —C(O)—;
one of L² and L³ represents —S(O)ᵣ—, and the other independently represents —OA²⁰-, —SC(R^{y3})(R^{y4})—, —S—, —S(O)—, —S(O)₂A¹⁸- or, preferably, —N(R^{w})A¹⁹-;
A¹⁶ represents a single bond or, preferably, —C(O)—;
A¹⁸ represents —N(R^{w})— or, preferably, a single bond;
A¹⁹ represents —C(R^{y3})(R^{y4})—, —C(O)O—, —C(O)C(R^{y3})(R^{y4})— or, preferably, a single bond, —C(O)—, —C(O)N(Rw)— or —S(O)₂—;
A²⁰ represents a single bond or —C(R^{y3})(R^{y4})—;
R^{y3} and R^{y4} independently represent H or X⁶, or, are linked together to form a 3-membered cyclopropyl group;
R^{w} represents H or X⁸;
X⁴ to X⁸ independently represent C₁₋₃ alkyl (e.g. methyl) optionally substituted by fluoro, or aryl (e.g. phenyl) optionally substituted by fluoro;
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} independently represent hydrogen or C₁₋₂ alkyl optionally substituted by ═O or, more preferably, one or more fluoro atoms.

More preferred compounds of the invention include those in which:

when ring A represents ring (I), in which there is one —N═ group present, then E^{a1}, E^{a3} or E^{a5} represents such a moiety;
when ring A represents ring (II), then W^{b} may represent —N(R^{3d})— (so forming a pyrrolyl or imidazolyl ring) or, more preferably, when Y^{b} represents —C(R^{3c})═, then W^{b} preferably represents —O— or, particularly, —S— (so forming a furyl or, particularly, a thienyl ring) or when Y^{b} represents —N═, then W^{b} preferably represents —O— or —S— (so forming, for example, an oxazolyl or thiazolyl ring);
R^{3c} and R^{3d} independently represent H;
when ring A represents ring (III), then W^{c} preferably represents —N(R^{4d})—;
R^{4d} represents H;
R^{8c}, R^{8f} and R^{8h} independently represent H or C₁₋₃ alkyl optionally substituted by one or more fluoro atoms;
X¹, X² and X³ independently represent fluoro, chloro, —CN, methyl, ethyl, isopropyl, difluoromethyl, trifluoromethyl, methoxy, ethoxy, difluoromethoxy and/or trifluoromethoxy;
R^{y1} and R^{y2} independently represent hydrogen;
A represents G¹ or C₁₋₆ alkyl (e.g. C₁₋₄ alkyl) optionally substituted by G¹ and/or Z¹.
A¹ represents —N(R^{17a})A⁴- or —OA⁵-;
G² represents halo or -A⁶-R^{18a}.

Preferred rings that ring A may represents include furyl (e.g. 2-furyl), thienyl (e.g. 2-thienyl), oxazolyl (e.g. 2-oxazolyl), thiazolyl (e.g. 2-thiazolyl), pyridyl (e.g. 2- or 4-pyridyl), pyrimininyl (e.g. 2-pyrimidinyl), pyrrolyl (e.g. 3-pyrrolyl), imidazolyl (e.g. 4-imidazolyl) or, preferably, phenyl.

Preferred rings that the D₁ to D₃-containing ring may represent include 2- or 4-pyridyl (relative to the point of attachment to the —C(O)— moiety) or, preferably, phenyl.

Preferred aryl and heteroaryl groups that Y² and Y³ may independently represent include optionally substituted (i.e. by A) pheny, naphthyl (e.g. 5,6,7,8-tetrahydronaphthyl), pyrrolyl, furyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include benzothienyl (e.g. 7-benzothienyl), 1,3-benzodioxolyl, particularly, naphthyl (e.g. 5,6,7,8-tetrahydronaphthyl or, preferably, 1-naphthyl or 2-naphthyl), more particularly, 2-benzoxazolyl, 2-benzimidazolyl, 2-benzothiazolyl, thienyl, oxazolyl, thiazolyl, pyridyl (e.g. 2- or 3-pyridyl), pyrrolyl (e.g. 2-pyrrolyl), imidazolyl (e.g. 1- or 2-imidazolyl) and, most preferably, phenyl.

Preferred substituents on Y² and Y³ groups (e.g. when they represent aryl or heteroaryl) include:

halo (e.g. fluoro, chloro or bromo);
cyano;
C₁₋₆ alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl);
heterocycloalkyl, such as a 5- or 6-membered heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR²⁶;

### —C(O)R²⁶;

### —C(O)OR²⁶;

### —N(R²⁶)R²⁷; and

—S(O)ₘR²⁶ (in which m is 0, or, preferably, 1 or 2);
wherein R²⁶ and R²⁷ independently represent, on each occasion when used herein, H, C₁₋₆ alkyl, such as C₁₋₄ alkyl (e.g. ethyl, n-propyl, t-butyl or, preferably, n-butyl, methyl or isopropyl) optionally substituted by one or more halo (e.g. fluoro) groups (so forming e.g. a perfluoroethyl or, preferably, a trifluoromethyl group) or aryl (e.g. phenyl) optionally substituted by one or more halo or C₁₋₃ (e.g. C₁₋₂) alkyl groups (which alkyl group is optionally substituted by one or more halo (e.g. fluoro) atoms). Preferably, when the substituent is —S(O)R²⁶ or —S(O)₂R²⁶, then R²⁶ does not represent hydrogen.

Preferred compounds of the invention include those in which:

D₁ and D₃ respectively represent —C(R^{1a})═ and —C(R^{1c})═;
D₂ₐ represents D₂;
D_{2b} represents —C(-L²-Y²)═;
D₂ represents —C(R^{1b})═;
R^{1a}, R^{1b} and R^{1c} independently represent H;
ring A represents ring (I);
E^{a1} and E^{a5} independently represent —C(H)═, or one or both of E^{a1} and E^{a5} may alternatively and independently represent —N═ (so forming for example, a phenyl, pyridyl (e.g. 2-pyridyl) or pyrimidinyl (e.g. 2-pyrimidinyl) group);
most preferably, E^{a1} and E^{a5} independently represent —C(H)═;
E^{a2}, E^{a3} and E^{a4} respectively represent —C(R^{2b})═, —C(R^{2c})═ and —C(R^{2d})═;
R^{2b} represents -L^{1a}-Y^{1a} or, more preferably, H;
R^{2c} represents the requisite -L³-Y³ group;
R^{2d} represents H;
L¹ represents a single bond;
Y¹ represents 5-tetrazolyl (which is preferably unsubstituted) or, preferably, —C(O)OR^{9a};
when Y¹ represents 5-tetrazolyl, then R^{2b} to R^{2d} (e.g. R^{2b}) do not represent -L^{1a}-Y^{1a} (but preferably represent hydrogen);
R^{9a} represents C₁₋₆ alkyl (e.g. ethyl or methyl) or, preferably, H;
L² represents —S(O)ᵣ—, and L³ represents —S—, —S(O)—, —S(O)₂—, preferably, —OA²⁰- or, more preferably, —N(R^{w})A¹⁹-;
L² and L³ may be different (for example when R^{2b} represents H) or L² and L³ are the same (for example when R^{2b} represents -L^{1a}-Y^{1a}); however, L² and L³ are preferably different;
A¹⁹ represents a single bond, —S(O)₂—, —C(O)— or —C(O)N(R^{w})—;
A²⁰ represents a single bond;
R^{w} represents C₁₋₃ alkyl (e.g. methyl) or H;
r represents 0, 1 or 2 (however, most preferably, r represents 1);
when Y² or Y³ represent optionally substituted C₁₋₁₂ alkyl, then it is preferably optionally substituted cycloalkyl (such as C₃₋₁₂ (e.g. C₃₋₈) cycloalkyl and, preferably, C₅₋₆ alkyl);
Y² and Y³ independently represent heteroaryl (such as 6-membered monocyclic heteroaryl group in which the heteroatom is preferably nitrogen or a 9-membered bicyclic heteroaryl group in which there is one or two heteroatom(s) preferably selected from nitrogen, sulfur and oxygen; so forming an oxazolyl, thiazolyl or thienyl group) optionally substituted as defined herein or, preferably, C₁₋₁₂ alkyl or aryl (e.g. naphthyl, such as 5,6,7,8-tetrahydronaphthyl, or, preferably, phenyl) both of which are optionally substituted as defined herein (most preferably, Y² and Y³ independently represent a cyclic group, e.g. an optionally substituted aryl or heteroaryl group as defined herein);
when Y² and Y³ represent optionally substituted aryl or heteroaryl groups, then there are preferably one or two optional substituents selected from A;
at least one of Y² and Y³ represents aryl (e.g. phenyl) optionally substituted as defined herein;
Y² and Y³ may be different (for example when R^{2b} represents H) or Y² and Y³ are the same (for example when R^{2b} represents -L^{1a}-Y^{1a});
when Y² or Y³ represent C₁₋₁₂ alkyl, then it is preferably a C₁₋₆ alkyl group (e.g. an unsubstituted acyclic C₁₋₆ alkyl group (such as n-hexyl), a part-cyclic C₁₋₆ alkyl group, such as cyclopentylmethyl, or, a cyclic C₃₋₆ alkyl group, such as cyclohexyl), optionally substituted by one or more G¹ substituent(s), in which G¹ is preferably -A¹-R^{16a}, A¹ is a single bond and R^{16a} is a (preferably unsubstituted) C₁₋₆ (e.g. C₁₋₄) alkyl group (e.g. tert-butyl) (most preferably, when Y² or Y³ represent C₁₋₁₂ alkyl, then it is an unsubstituted acyclic C₁₋₆ alkyl group, such as n-hexyl);
A represents G¹ or C₁₋₆ (e.g. C₁₋₄) alkyl (e.g. butyl (such as n-butyl) or methyl) optionally substituted by one or more substituents selected from G¹;
G¹ represents halo (e.g. chloro or fluoro, when G¹ is attached to an aromatic ring, or, fluoro, when G¹ is attached to a non-aromatic group, e.g. an alkyl group) or -A¹-R^{16a};
A¹ represents a single bond or, preferably, —OA⁵-;
A⁵ represents a single bond;
R^{16a} represents hydrogen or C₁₋₆ (e.g. C₁₋₄) alkyl optionally substituted by one or more substituents selected from G³ (e.g. R^{16a} may represent ethyl or, preferably, butyl (such as tert-butyl or, preferably n-butyl), propyl (such as isopropyl) or methyl);
G³ represents halo (e.g. fluoro; and hence e.g. R^{16a} may represent trifluoromethyl or perfluoroethyl);
when Y² and/or Y³ represent an optionally substituted phenyl group, then that phenyl group may be substituted with a single substituent (e.g. at the meta or para-position) or with two substituents (e.g. with one at the para-position and the other at the meta- or ortho- (3- or 2-) position, so forming for example a 2,4-substituted, 2,5-substituted or, preferably, a 3,4-substituted phenyl group);
R²⁸ represents hydrogen or unsubstituted C₁₋₃ (e.g. C₁₋₂) alkyl (e.g. methyl).

Most preferred compounds of the invention include those in which:

Y represents —C(O)—;
none of R^{2b} to R^{2d} represents -L^{1a}-Y^{1a};
ring A represents ring I);
D₁, D₂ₐ, D₃, E^{a2} and E^{a4} independently represent —C(H)═ (hence D_{2b} represents —C(-L²-Y²)═ and E^{a3} represents —C(-L³-Y³)═);
L² represents —S(O)ᵣ—, and L³ represents —N(R^{w})A¹⁹-;
A¹⁹ represents a single bond;
R^{w} represents hydrogen or, preferably C₁₋₃ (e.g. C₁₋₂) alkyl (e.g. methyl);
r represents 1;
when L² represents —S(O)ᵣ—, then Y² represents optionally substituted C₁₋₁₂ alkyl (as defined herein) or aryl or heteroaryl (preferably aryl; but which two groups are also optionally substituted as defined herein);
when L³ represents the a single bond or a linker moiety (e.g. —OA²⁰- or, preferably, —N(R^{w})A¹⁹-), then Y³ represents aryl or heteroaryl (preferably aryl; but which two groups are also optionally substituted as defined herein);
A represents G¹;
G¹ represents halo (e.g. chloro or fluoro, for example when A represents G¹).

It is preferred than, when L² represents —S(O)ᵣ— (e.g. —S(O)—), then L³ is selected from —S(O)— or, preferably, a single bond —(CH₂)ₚ—C(R^{y3})(R^{y4})—(CH₂)_{q}-A¹⁶-, —C(O)A¹⁷-, —N(R^{w})A¹⁹- and —OA²⁰-. The most preferred compounds of the invention include those in which L² represents —S(O)—, and L³ is as hereinbefore defined (e.g. —OA²⁰- or, preferably, —N(R^{w})A¹⁹-).

Preferred substituents on Y² or Y³ groups (for instance, when they represent heteroaryl groups or, preferably, aryl group, such as phenyl) include halo (e.g. chloro and fluoro).

When Y² or Y³ represents optionally substituted C₁₋₁₂ alkyl, then that group is preferably C₁₋₈ (e.g. C₁₋₈) alkyl, especially linear alkyl, such as hexyl (e.g. n-hexyl), which group is preferably unsubstituted.

Particularly preferred compounds of the invention include those of the following formula:

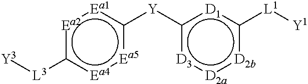

wherein:
either one of D₂ₐ and D_{2b} represents D₂, and the other represents —C(-L²-Y²)═ and hence the compound may be either one of the following two formulae:

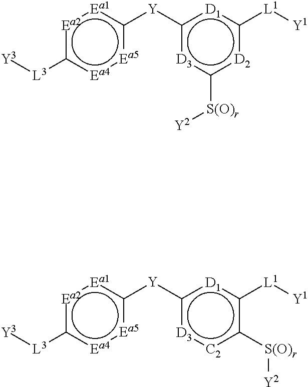

wherein:
r represents 0, 1 or 2;
Y represents —C(O)— or —C(═N—OR²⁸)—;
R²⁸ represents hydrogen or C₁₋₃ (e.g. C₁₋₂) alkyl;
each of D₁, D₂ and D₃ respectively represent —C(R^{1a})═, —C(R^{1b})═ and —C(R^{1c})═;
R^{1a}, R^{1b} and R^{1c} independently represent R^{5a}, halo, —CN or, preferably, hydrogen;
at least one (e.g. at least two, and preferably all) of R^{1a}, R^{1b} and R^{1c} represent hydrogen;
each of E^{a1}, E^{a2}, E^{a4} and E^{a5} respectively represent —C(H)═, —C(R^{2b})═, —C(R^{2d})═ and —C(H)═, or, one or two (e.g. one) of E^{a1}, E^{a2}, E^{a4} and E^{a5} (e.g. E^{a1} and/or E^{a5}; preferably either E^{a1} or E^{a5}) may alternatively and independently represent —N═ (hence, this ring is preferably phenyl, pyridyl, such as 2-pyridyl, or pyrimidinyl, such as 2-pyrimidinyl);
R^{2b} and R^{2d} independently represent a substituent selected from X¹ or, more preferably, hydrogen;
X¹, X² and X³ independently represent a group selected from R^{5a}, halo or —CN;
R^{5a} represents, on each occasion when used herein, C₁₋₆ (e.g. C₁₋₄) alkyl optionally substituted by one or more substituents selected from ═O and, preferably, halo, —CN and —N₃ (e.g. halo and —CN);
Y¹ and Y^{1a} independently represent, on each occasion when used herein, —C(O)OR^{9a};
R^{9a} represents hydrogen or C₁₋₆ (e.g. C₁₋₄) alkyl;
Y² represents: (i) 5- or, preferably, 6-membered heteroaryl (e.g. in which there is preferably one heteroatom, preferably selected from nitrogen, oxygen and sulfur); (ii) 9- or 10-membered bicyclic heteroaryl group (e.g. consisting of a benzene ring fused to a 5- or 6-membered heteroaryl or heterocycloalkyl group); (iii) or a 4- to 8-membered (e.g. 5- or 6-membered) heterocycloalkyl group; or Y² more preferably represents (iv) acyclic C₁₋₆ (e.g. C₄₋₆) alkyl (e.g. butyl or hexyl); (v) phenyl; or (vi) C₃₋₁₀ (e.g. C₅₋₆ monocyclic cycloalkyl or C₈₋₁₀ polycyclic or bridged cycloalkyl)cycloalkyl (e.g. cyclopentyl, cyclohexyl, adamantyl (e.g. 1-adamantyl) or bicyclo[2.2.1]heptyl (e.g. 1-bicyclo[2.2.2]heptyl)), all of which groups are optionally substituted by one or more substituents selected from A (or the alkyl and heterocycloalkyl groups may be optionally substituted by one or more substituents selected from G¹ and Z¹);
Y³ may represents a group as defined above for Y² (provided that at least one of Y² and Y³ represents an aromatic group), but Y³ preferably represents aryl (e.g. phenyl) optionally substituted by one or more substituents selected from A;
A represents G¹ or C₁₋₄ alkyl optionally substituted by one or more substituents selected from G¹ (e.g. fluoro; so forming e.g. a trifluoromethyl group);
when Y² or Y³ (e.g. Y²) represents acyclic C₁₋₆ alkyl substituted by A, then A may also represent aryl (e.g. phenyl) or C₄₋₈ cycloalkyl (e.g. bicycloheptyl, such as bicycle[2.2.1]-1-heptyl), both of which are optionally substituted by one or more substituents selected from G¹ and G² (as appropriate; e.g. C₁₋₄ alkyl and/or —OR^{16a} (or —OR^{18a}), such as methyl, —OH and/or —OC₁₋₃ alkyl);
G¹ represents halo (e.g. chloro, fluoro or bromo) or -A¹-R^{16a};
A¹ represents a single bond or —OA⁵-;
A⁵ represents a single bond;
G² represents halo (e.g. chloro, fluoro or bromo) or -A⁶-R^{18a};
A⁶ represents —OA¹⁰-;
A¹⁰ represents a single bond;
R^{16a} and R^{18a} independently represent hydrogen or C₁₋₄ (e.g. C₁₋₂) alkyl (e.g. methyl) optionally substituted by one or more fluoro atoms (so forming e.g. a trifluoromethyl group);
G³ represents halo (e.g. fluoro);
L¹ (and L^{1a}, if present) independently represent a single bond;
L² represents —S(O)ᵣ—CH₂— (in which r is preferably 0 or 1) or L² more preferably represents —S(O)ᵣ— (in which r may represent 0, 1 or 2);
L³ represents —N(R^{w})A¹⁹- or —OA²⁰-;
A¹⁹ represents a single bond;
A²⁰ represents a single bond or, e.g. preferably, —C(R^{y3})(R^{y4})—;
R^{y3} and R^{y4} independently represent hydrogen;
R^{w} represents, on each occasion when used herein, H or X⁸;
X⁸ represents C₁₋₆ (e.g. C₁₋₄) alkyl (e.g. methyl or cyclopropylmethyl).

For the avoidance of doubt, all individual features (e.g. preferred features) mentioned herein may be taken in isolation or in combination with any other feature (including preferred feature) mentioned herein (hence, preferred features may be taken in conjunction with other preferred features, or independently of them).

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which Y represents —C(O)—, oxidation of a compound of formula II,

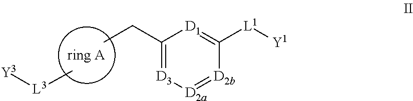

wherein ring A, D₁, D₂ₐ, D_{2b}, D₃, L¹, Y¹, L³ and Y³ are as hereinbefore defined, in the presence of a suitable oxidising agent (although, the skilled person will appreciate that certain other moieties, e.g. —S— moieties, may simultaneously be oxidised);
(ia) for compounds of formula I in which Y represents —C(O)—, oxidation of a compound of formula IIA,

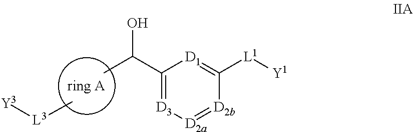

wherein ring A, D₁, D₂ₐ, D_{2b}, D₃, L¹, Y¹, L³ and Y³ are as hereinbefore defined, in the presence of a suitable oxidising agent, for example, pyridinium chlorochromate (PCC) or the like (e.g. pyridinium dichromate; PDC);
(ii) for compounds of formula I in which L³ represents —N(R^{w})A¹⁹- in which R^{w} represents H (and, preferably, Y is —C(O)— or R²⁸ is C₁₋₆ alkyl optionally substituted by one or more halo atoms), reaction of a compound of formula III,

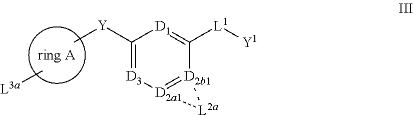

or a protected derivative thereof (e.g. an amino-protected derivative or a keto-protecting group, such as a ketal or thioketal) wherein one of D₂ₐ₁ and D_{2b1} represents D₂ and the other represents —C(-L^{2a})═ (i.e. the L^{2a} substituent is attached to either one of D₂ₐ₁ and D_{2b1}), L^{2a} represents —S(O)ᵣ—Y², L^{3a} represents —NH₂, and Y, ring A, D₁, D₂, D₃, L¹ and Y¹ are as hereinbefore defined, with:
(A) when A¹⁹ represents —C(O)N(R^{w})—, in which R^{w} represents H:
(a) a compound of formula IV,

Y^{a}—N═C═O IV;
or
(b) with CO (or a reagent that is a suitable source of CO (e.g. Mo(CO)₆ or CO₂(CO)₈)) or a reagent such as phosgene or triphosgene in the presence of a compound of formula V,

Y^{a}—NH₂ V

wherein, in both cases, Y^{a} represents Y² or Y³ (as appropriate/required; in this case, Y^{a} necessarily represents Y³) as hereinbefore defined. For example, in the case of (a) above, in the presence of a suitable solvent (e.g. THF, dioxane or diethyl ether) under reaction conditions known to those skilled in the art (e.g. at room temperature). In the case of (b), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). For the preparation of such compounds protection (e.g. at an amino group) followed by deprotection may be necessary, or the reaction may be performed with less than 2 equivalents of the compound of formula IV or V (as appropriate);
(B) when A¹⁹ represents —S(O)₂N(R^{w})—:
(a) ClSO₃H, followed by PCl₅, and then reaction with a compound of formula V as hereinbefore defined;
(b) SO₂Cl₂, followed by reaction with a compound of formula V as hereinbefore defined;
(c) a compound of formula VA,

Y^{a}—N(H)SO₂Cl VA
wherein Y^{a} is as hereinbefore defined;
(d) ClSO₂N═C═O, optionally in the presence BrCH₂CH₂OH, following by reaction in the presence of a compound of formula V as hereinbefore defined (which reaction may proceed via a 2-oxazolidinone intermediate),
   for example under standard reaction conditions, for e.g. such as those described hereinbefore in respect of process step (ii)(A) above (e.g. employing a Cu or Pd catalyst under Goldberg coupling or Buchwald-Hartwig reaction conditions), followed by standard oxidation reaction conditions (for example, reaction in the presence of an oxidising reagent such as meta-chloroperbenzoic acid in the presence of a suitable solvent such as dichloromethane e.g. as described in Journal of Organic Chemistry, (1988) 53(13), 3012-16, or, KMnO₄, e.g. as described in Journal of Organic Chemistry, (1979), 44(13), 2055-61. The skilled person will also appreciate that the compound of formula VA may need to be prepared, for example from a corresponding compound of formula V as defined above, and SO₂ (or a suitable source thereof) or SOCl₂;
   (C) when A¹⁹ represents a single bond, with a compound of formula VI,

Y^{a}-L^{a} VI

wherein L^{a} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group) and Y^{a} is as hereinbefore defined, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Y^{a} represents phenyl and L^{a} represents bromo, i.e. bromobenzene). This reaction may be carried out at room temperature or above (e.g. at a high temperature, such as the reflux temperature of the solvent system that is employed) or using microwave irradiation;
(D) when A¹⁹ represents —S(O)₂—, —C(O)—, —C(R^{y3})(R^{y4})—, —C(O)—C(R^{y3})(R^{y4})— or —C(O)O—, with a compound of formula VII,

Y^{a}-A^{19a}-L^{a} VII

wherein A^{19a} represents —S(O)₂—, —C(O)—, —C(R^{y3})(R^{y4})—, —C(O)—C(R^{y3})(R^{y4})— or —C(O)O—, and Y^{a} and L^{a} are as hereinbefore defined, and L^{a} is preferably, bromo or chloro, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potass-ium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);
(iii) for compounds of formula I in which L³ represents —N(R^{w})C(O)N(R^{w})—, in which R^{w} represents H (in both cases), and, preferably, Y is —C(O)— or R²⁸ is C₁₋₆ alkyl optionally substituted by one or more halo atoms, reaction of a compound of formula VIII,

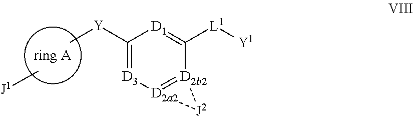

wherein one of D₂ₐ₂ and D_{2b2} represents D₂ and the other represents —C(-J²)= (i.e. the J² substituent is attached to either one of D₂ₐ₂ and D_{2b2}), one of J¹ represents —N═C═O and J² represents —S(O)ᵣ—Y², and Y, ring A, D₁, D₂ₐ, D_{2b}, D₃, L¹ and Y¹ are as hereinbefore defined, with a compound of formula V as hereinbefore defined, under reaction conditions known to those skilled in the art, such as those described hereinbefore in respect of process step (ii)(A)(b) above;
(iv) for compounds of formula I in which, preferably, Y is —C(O)— or R²⁸ is C₁₋₆ alkyl optionally substituted by one or more halo atoms (this is particularly preferred for the preparation of compounds of formula I in which either of L² and L³ represents —S—), reaction of a compound of formula IX,

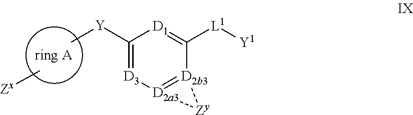

wherein one of D₂ₐ₃ and D_{2b3} represents D₂ and the other represents —C(—Z^{y})═ (i.e. the Z^{y} substituent is attached to either one of D₂ₐ₃ and D_{2b3}), wherein at least one of Z^{x} and Z^{y} represents a suitable leaving group and the other may also independently represent a suitable leaving group, or, Z^{y} may represent -L²-Y² and Z^{x} may represent -L³-Y³, in which the suitable leaving group may independently be fluoro or, preferably, chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and Y, ring A, D₁, D₂ₐ, D_{2b}, D₃, L¹, Y¹, L², Y², L³ and Y³ are as hereinbefore defined, with a (or two separate) compound(s) (as appropriate/required) of formula X,

Y^{a}-L^{x}-H X

wherein L^{x} represents L² or L³ (as appropriate/required), and Y^{a} is as hereinbefore defined, under suitable reaction conditions known to those skilled in the art, for example such as those hereinbefore described in respect of process (ii) above (e.g. process (ii)(B) or (ii)(C)), for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). Alternatively, for example, when L³ represents —O— (and hence the compound of formula X is an alcohol, e.g. a phenol) or L² and/or L³ represents —S—, (i.e. the compound of formula X is a thiol, e.g. a thiophenol), then the reaction may be performed in the presence of a mixture of KF/Al₂O₃ (e.g. in the presence of a suitable solvent such as acetonitrile, at elevated temperature, e.g. at about 100° C.; in this instance the leaving group that Z^{x} or Z^{y} may represent in the compound of formula IX is preferably fluoro). Alternatively still, when L^{x} represents —S(O)₂A¹⁸-, in which A¹⁸ represents —N(R^{w})—), Ullman reaction conditions such as those described in Tetrahedron Letters, (2006), 47(28), 4973-4978 may be employed. The skilled person will appreciate that when compounds of formula I in which L² and L³ are different are required, then reaction with different compounds of formula X (for example, first reaction with a compound of formula X in which L^{x} represents —S(O)ᵣ— (in which r is preferably 0), followed by reaction with another, separate, compound of formula X in which L^{x} represents —N(R^{w})A¹⁹- or —S(O)₂A¹⁸-) may be required;
(v) for compounds of formula I in which L² represents —S(O)ᵣ— in which r is 1 or 2, and, optionally (or alternatively), L³ represents —S(O)— or —S(O)₂— (and Y is, preferably, —C(O)— or R²⁸ is C₁₋₆ alkyl optionally substituted by one or more halo atoms), oxidation of a corresponding compound of formula I in which L² and/or L³ represents —S— (or —S(O)—, for the preparation of compounds of formula I in which there is a —S(O)₂— moiety present), under standard conditions, for example in the presence of a suitable oxidising agent, such as Oxone or meta-chloroperbenzoic acid (MCPBA), optionally in the presence of a suitable solvent);
(vi) for compounds of formula I in which L² and/or L³ represents —S—, Y² and Y³ are preferably optionally substituted aryl or heteroaryl (Y is, preferably, —C(O)— or R²⁸ is C₁₋₆ alkyl optionally substituted by one or more halo atoms), reaction of a compound of formula IX, wherein at least one of Z^{x} and Z^{y} represents a suitable leaving group (preferably iodo) and the other may also independently represent a suitable leaving group, or, Z^{y} may represent -L²-Y² and Z^{x} may represent -L³-Y³, with a (or two separate) compound(s) (as appropriate/required; only half an equivalent may be required) of formula XI

Y^{a}—S—S—Y^{a} XI

wherein Y^{a} is as hereinbefore defined (hence, for the introduction of —S—Y², the compound of formula XI is Y²—S—S—Y², in which Y² is preferably optionally substituted aryl or heteroaryl), under suitable reaction conditions known to those skilled in the art, for example in the presence of a suitable catalyst, for example a nickel-based catalyst (e.g. NiBr₂), for example as described in J. Org. Chem. (2004), 69, 6904-6906;
(vii) for compounds of formula I in which L² and/or L³ represents —S(O)₂—, reaction of a compound of formula XII,

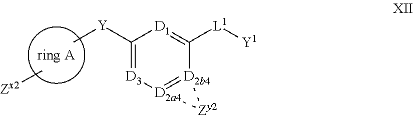

wherein one of D₂ₐ₄ and D_{2b4} represents D₂ and the other represents —C(—Z^{y2})═ (i.e. the Z^{y2} substituent is attached to either one of D₂ₐ₄ and D_{2b4}), wherein at least one of Z^{x2} and Z^{y2} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L²-Y² or -L³-Y³ (as appropriate), and Y, ring A, D₁, D₂, D₃, L¹ and Y¹ are as hereinbefore defined, with a (or two separate) compound(s) (as appropriate/required) of formula XIII,

Y^{a}—S(O)₂-L^{x1} XIII

wherein L^{x1} represents a suitable leaving group known to those skilled in the art, such as iodo, and, especially chloro or bromo, and Y^{a} is as hereinbefore defined, under suitable reaction conditions known to those skilled in the art, for example such as those hereinbefore described, e.g. in respect of process step (ii) above;
(viii) for compounds of formula I in which L² and/or L³ represents —S(O)—, reaction of a compound of formula IX with a (or two separate) compound(s) (as appropriate/required) of formula XIV,

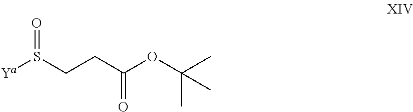

wherein Y^{a} is as hereinbefore defined, under suitable reaction conditions known to those skilled in the art, for example under reaction conditions such as those described in Org. Lett., 2006, 8, 5951-5954;
(ix) compounds of formula I in which there is a R^{w} group present that does not represent hydrogen (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen), may be prepared by reaction of a corresponding compound of formula I in which such a group is present that does represent hydrogen with a compound of formula XV,

R^{wy}-L^{b} XV

wherein R^{wy} represents either R^{w} (as appropriate) as hereinbefore defined provided that it does not represent hydrogen (or R^{w} represents a R⁵ to R²⁶ group in which those groups do not represent hydrogen), and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (ii) above (e.g. step (ii)(D)). The skilled person will appreciate that various groups (e.g. primary amino groups) may need to be mono-protected and then subsequently deprotected following reaction with the compound of formula XV;
(x) for compounds of formula I that contain only saturated alkyl groups, reduction of a corresponding compound of formula I that contains an unsaturation, such as a double or triple bond, in the presence of suitable reducing conditions, for example by catalytic (e.g. employing Pd) hydrogenation;
(xi) for compounds of formula I in which Y¹ and/or, if present, Y^{1a} represents —C(O)OR^{9a}, in which R^{9a} represents hydrogen (or, other carboxylic acid or ester protected derivatives (e.g. amide derivatives)), hydrolysis of a corresponding compound of formula I in which R^{9a} does not represent H, under standard conditions, for example in the presence of an aqueous solution of base (e.g. aqueous 2M NaOH) optionally in the presence of an (additional) organic solvent (such as dioxane or diethyl ether), which reaction mixture may be stirred at room or, preferably, elevated temperature (e.g. about 120° C.) for a period of time until hydrolysis is complete (e.g. 5 hours). Alternatively, non-hydrolytic means may be employed to convert esters to acids e.g. by hydrogentation or oxidation (e.g. for certain benzylic groups) known to those skilled in the art;
(xii) for compounds of formula I in which Y¹ and/or, if present, Y^{1a} represents —C(O)OR^{9a}, and R^{9a} does not represent H:
(A) esterification (or the like) of a corresponding compound of formula I in which R^{9a} represents H; or
(B) trans-esterification (or the like) of a corresponding compound of formula I in which R^{9a} does not represent H (and does not represent the same value of the corresponding R^{9a} group in the compound of formula I to be prepared),
   under standard conditions in the presence of the appropriate alcohol of formula XVI,

R^{9za}OH XVI

in which R^{9za} represents R^{9a} provided that it does not represent H, for example further in the presence of acid (e.g. concentrated H₂SO₄) at elevated temperature, such as at the reflux temperature of the alcohol of formula XVII;
(xiii) for compounds of formula I in which Y¹ and/or, if present, Y^{1a} represents —C(O)OR^{9a}, in which R^{9a} is other than H, and L¹ and/or, if present, L^{1a}, are as hereinbefore defined, provided that they do not represent —(CH₂)ₚ-Q-(CH₂)_{q}— in which p represents 0 and Q represents —O—, and, preferably, Y is —C(O)— or R²⁸ is C₁₋₆ alkyl optionally substituted by one or more halo atoms, reaction of a compound of formula XVII,

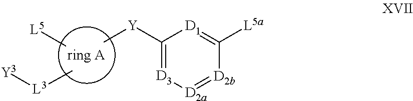

wherein at least one of L⁵ and L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L₁ₐ-Y^{1a} (as appropriate), and Y, ring A, D₁, D₂ₐ, D_{2b}, D₃, L³ and Y³ are as hereinbefore defined (the skilled person will appreciate that the compound of formula XVII in which L⁵ and/or L^{5a} represents an alkali metal (e.g. lithium), a Mg-halide or a zinc-based group may be prepared from a corresponding compound of formula XVII in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XVIII,

L⁶⁻L^{xy}-Y^{b} XVIII

wherein L^{xy} represents L¹ or L^{1a} (as appropriate; provided that it does not represent —(CH₂)ₚ-Q-(CH₂)_{q}— in which p represents 0 and Q represents —O—) and Y^{b} represents —C(O)OR^{9a}, in which R^{9a} is other than H, and L⁶ represents a suitable leaving group known to those skilled in the art, such as C₁₋₃ alkoxy or, preferably, halo (especially chloro or bromo). For example, for compounds of formula I in which L¹ represents a single bond and Y¹ represents —C(O)OR^{9a}, the compound of formula XVIII may be Cl—C(O)OR^{9a}. The reaction may be performed under standard reaction conditions, for example in the presence of a polar aprotic solvent (e.g. THF or diethyl ether);
(xiv) compounds of formula I in which L¹ and/or, if present, L^{1a} preferably represent a single bond, and Y¹ and/or, if present, Y^{1a} represents 5-tetrazolyl, may be prepared in accordance with the procedures described in international patent application WO 2006/077366;
(xv) for compounds of formula I in which L¹ and/or, if present, L^{1a} represent a single bond, and Y¹ and/or, if present, Y^{1a} represent —C(O)OR^{9a} in which R^{9a} is H, (and, preferably, Y is —C(O)— or R²⁸ is C₁₋₆ alkyl optionally substituted by one or more halo atoms), reaction of a compound of formula XVII as hereinbefore defined but in which L⁵ and/or L^{5a} (as appropriate) represents either:
(I) an alkali metal (for example, such as one defined in respect of process step (xv) above); or
(II) —Mg-halide,
   with carbon dioxide, followed by acidification under standard conditions known to those skilled in the art, for example, in the presence of aqueous hydrochloric acid;
   (xvi) for compounds of formula I in which L¹ and/or, if present, L^{1a} represent a single bond, and Y¹ and/or, if present, Y^{1a} represent —C(O)OR^{9a} (and, preferably, Y is —C(O)— or R²⁸ is C₁₋₆ alkyl optionally substituted by one or more halo atoms), reaction of a corresponding compound of formula XVII as hereinbefore defined but in which L⁵ and/or L^{5a} (as appropriate) is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈)), in the presence of a compound of formula XIX,

R^{9a}OH XIX

wherein R^{9a} is as hereinbefore defined, and an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like) under conditions known to those skilled in the art;
(xvii) for compounds of formula I in which Y represents —C(O)—, reaction of either a compound of formula XX or XXI,

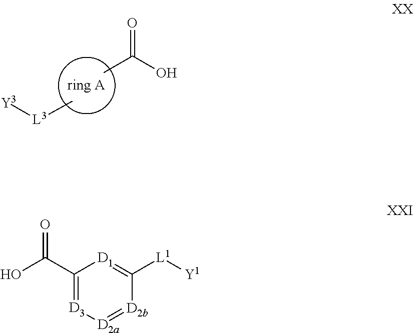

respectively with a compound of formula XXII or XXIII,

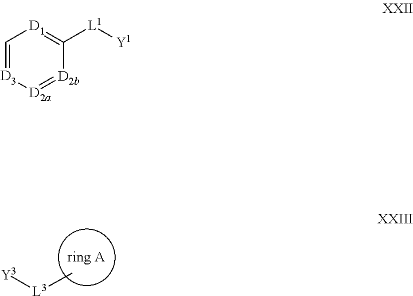

wherein (in all cases) ring A, D₁, D₂ₐ, D_{2b}, D₃, L¹, Y¹, L³ and Y³ are as hereinbefore defined, in the presence of a suitable reagent that converts the carboxylic acid group of the compound of formula XXI or XXII to a more reactive derivative (e.g. an acid chloride or acid anhydride, or the like; which reactive derivative may itself be separately prepared and/or isolated, or where such a reactive derivative may be prepared in situ) such as POCl₃, in the presence of ZnCl₂, for example as described in Organic and Biomolecular Chemistry (2007), 5(3), 494-500 or, more preferably, PCl₃, PCl₅, SOCl₂ or (COCl)₂. Alternatively, such a reaction may be performed in the presence of a suitable catalyst (for example a Lewis acid catalyst such as SnCl₄), for example as described in Journal of Molecular Catalysis A: Chemical (2006), 256(1-2), 242-246 or under alternative Friedel-crafts acylation reaction conditions (or variations thereupon) such as those described in Tetrahedron Letters (2006), 47(34), 6063-6066; Synthesis (2006), (21), 3547-3574; Tetrahedron Letters (2006), 62(50), 11675-11678; Synthesis (2006), (15), 2618-2623; Pharmazie (2006), 61(6), 505-510; and Synthetic Communications (2006), 36(10), 1405-1411. Alternatively, such a reaction between the two relevant compounds may be performed under coupling reaction conditions (e.g. Stille coupling conditions), for example as described in Bioorganic and Medicinal Chemistry Letters (2004), 14(4), 1023-1026;
(xviii) for compounds of formula I in which Y represents —C(O)—, reaction of either a compound of formula XXIV or XXV,

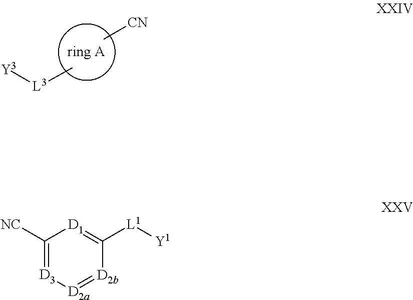

with a compound of formula XXVI or XXVII,

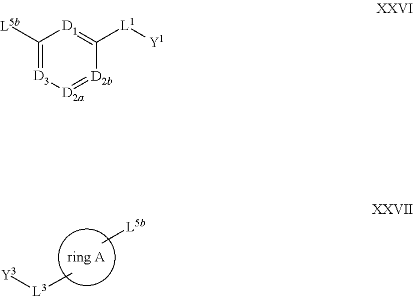

respectively, wherein L^{5b} represents L⁵ as hereinbefore defined provided that it does not represent -L¹-Y¹ or -L^{1a}-Y^{1a} (i.e. it represents a suitable leaving group as defined in respect of L⁵), and which L^{5b} group may therefore represent —B(OH)₂ (or a protected derivative thereof), an alkali metal (such as lithium) or a —Mg-halide (such as —MgI or, preferably, —MgBr), and (in all cases) ring A, D₁, D₂ₐ, D_{2b}, D₃, L¹, Y¹, L³ and Y³ are as hereinbefore defined, for example in the presence of a suitable solvent, optionally in the presence of a catalyst, for example, as described in Organic Letters (2006), 8(26), 5987-5990. Compounds of formula I may also be obtained by performing variations of such a reaction, for example by performing a reaction of a compound of formula XXIV or XXV respectively with a compound of formula XXII or XXIII as hereinbefore defined, for example under conditions described in Journal of Organic Chemistry (2006), 71(9), 3551-3558 or US patent application US 2005/256102;
(xix) for compounds of formula I in which Y represents —C(O)—, reaction of an activated derivative of a compound of formula XX or XXI as hereinbefore defined (for example an acid chloride; the preparation of which is hereinbefore described in process step (xvii) above), with a compound of formula XXVII or XXVIII (as hereinbefore defined), respectively, for example under reaction conditions such as those hereinbefore described in respect of process step (xviii) above;
(xx) for compounds of formula I in which Y represents —C(═N—OR²⁸)—, reaction of a corresponding compound of formula I, with a compound of formula XXVIII,

H₂N—O—R²⁸ XXVIII

wherein R²⁸ is represents hydrogen or C₁₋₆ alkyl optionally substituted by one or more halo atoms, under standard condensation reaction conditions, for example in the presence of an anhydrous solvent (e.g. dry pyridine, ethanol and/or another suitable solvent);
(xxi) for compounds of formula I in which Y represents —C(═N—OR²⁸)— and R²⁸ represents C₁₋₆ alkyl optionally substituted by one or more halo atoms, reaction of a corresponding compound of formula I, in which R²⁸ represents hydrogen, with a compound of formula XXVIIIB,

R^{28a}-L⁷ XXVIIIB

wherein R^{28a} represents R²⁸, provided that it does not represent hydrogen and L⁷ represents a suitable leaving group, such as one hereinbefore defined in respect of L^{a} (e.g. bromo or iodo), under standard alkylation reaction conditions, such as those hereinbefore described in respect of process step (ii) (e.g. (ii)(C)).

Compounds of formula II may be prepared by reaction of a compound of formula XXII with a compound of formula XXIII, both as hereinbefore defined, with formaldehyde (e.g. in the form of paraformaldehyde or an aqueous solution of formaldehyde such as a 3% aqueous solution), for example under acidic conditions (e.g. in the presence of aqueous HCl) at or above room temperature (e.g. at between 50° C. and 70° C.). Preferably, the formaldehyde is added (e.g. slowly) to an acidic solution of the compound of formula XXIII at about 50° C., with the reaction temperature rising to about 70° C. after addition is complete. When acidic conditions are employed, precipitation of the compound of formula II may be effected by the neutralisation (for example by the addition of a base such as ammonia). Compounds of formula I may also be prepared in accordance with such a procedure, for example under similar reaction conditions, employing similar reagents and reactants.

Compounds of formula IIA may be prepared by reaction of a compound of formula XXVIIIC or XXVIIID,

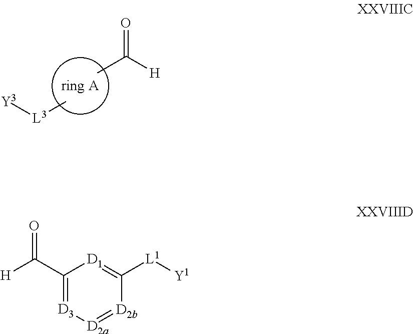

wherein ring A, D₁, D₂ₐ, D_{2b}, D₃, L¹, L³, Y¹ and Y³ are as hereinbefore defined, with a compound of formula XXVI or XXVII, respectively, for example under reaction conditions such as those hereinbefore described in respect of preparation of compounds of formula I (process step (xviii)).

Compounds of formulae III, VIII, IX and XVII in which Y represents —C(O)—, may be prepared by oxidation of a compound of formulae XXIX, XXX, XXXI and XXXII, respectively,

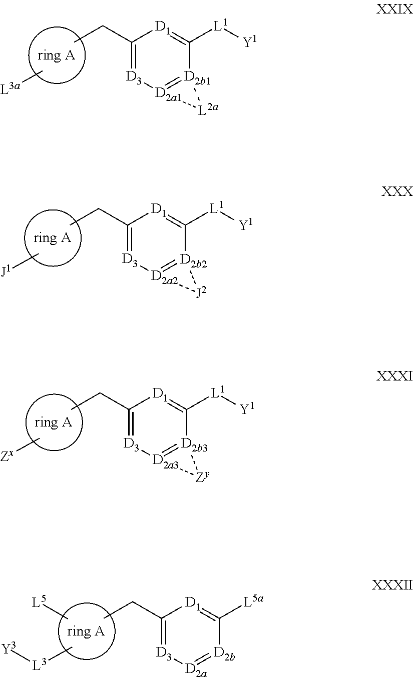

wherein ring A, D₁, D₂ₐ, D_{2b}, D₂ₐ₁, D_{2b1}, D₂ₐ₂, D_{2b2}, D₂ₐ₃, D_{2b3}, D₃, L¹, Y¹, L^{2a}, L^{3a}, Z^{x}, Z^{y}, L², Y², L³, Y³, J¹, J², L⁵ and L^{5a} are as hereinbefore defined, under standard oxidation conditions known to those skilled in the art, for example such as those hereinbefore described in respect of preparation of compounds of formula I (process step (i) above). The skilled person will appreciate that, similarly, compounds of formulae XXIX, XXX, XXXI and XXXII may be prepared by reduction of corresponding compounds of formulae III, VIII, IX and XVII, under standard reaction conditions, such as those described herein.

Compounds of formula III in which Y represents —C(O)—, or, preferably, compounds of formula XXIX (or protected, e.g. mono-protected derivatives thereof) may be prepared by reduction of a compound of formula XXXIII,

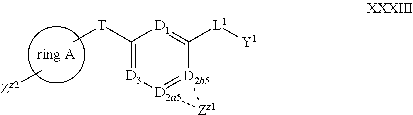

wherein one of D₂ₐ₅ and D_{2b5} represents D₂ and the other represents —C(—Z^{z1})═ (i.e. the Z^{z1} substituent is attached to either one of D₂ₐ₅ and D_{2b5}), T represents —C(O)— (in the case where compounds of formula III are to be prepared) or, preferably, —CH₂— (in the case where compounds of formula XXIX are to be prepared), one of Z^{z1} and Z^{z2} represents —N₃ or —NO₂, and the other represents —S(O)ᵣ—Y² or —S(O)ᵣ—Y³ (as appropriate), under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane). The skilled person will appreciate that where the reduction is performed in the presence of a —C(O)— group (e.g. when T represents —C(O)—), a chemoselective reducing agent may need to be employed.

Compounds of formula III in which L^{2a} or L^{3a} represent —NH₂ (or protected derivatives thereof) may also be prepared by reaction of a compound of formula IX as defined above, with ammonia, or preferably with a protected derivative thereof (e.g. benzylamine or Ph₂C═NH), under conditions such as those described hereinbefore in respect of preparation of compounds of formula I (process step (iv) above).

Compounds of formulae III, IX, XXIX or XXXI in which L¹ represents a single bond, and Y¹ represents —C(O)OR^{9a}, may be prepared by:

(I) reaction of a compound of formula XXXIV,

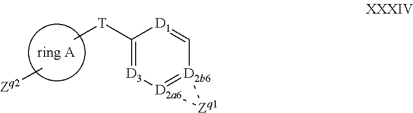

wherein one of D₂ₐ₆ and D_{2b6} represents D₂ and the other represents —C(—Z^{q1})═ (i.e. the Z^{q1} substituent is attached to either one of D₂ₐ₆ and D_{2b6}), Z^{q1} and Z^{q2} respectively represent Z^{x} and Z^{y} (in the case of preparation of compounds of formulae IX or XXXI) or L^{2a} and L^{3a} (in the case of preparation of compounds of formulae III or XXIX), and ring A, D₁, D₂, D₃, Z^{x}, Z^{y}, L^{2a}, L^{3a} and T are as hereinbefore defined, with a suitable reagent such as phosgene or triphosgene in the presence of a Lewis acid, followed by reaction in the presence of a compound of formula XIX as hereinbefore defined, hence undergoing a hydrolysis or alcoholysis reaction step;
(II) for such compounds in which R^{9a} represents hydrogen, formylation of a compound of formula XXXIV as hereinbefore defined, for example in the presence of suitable reagents such as P(O)Cl₃ and DMF, followed by oxidation under standard conditions;
(III) reaction of a compound of formula XXXV,

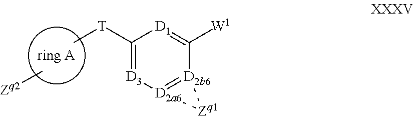

wherein W¹ represents a suitable leaving group such as one defined by Z^{x} and Z^{y} above, and ring A, D₁, D₂ₐ₆, D_{2b6}, D₃, Z^{q1}, Z^{q2} and T are as hereinbefore defined, are as hereinbefore defined, with CO (or a reagent that is a suitable source of CO (e.g. Mo(CO)₆ or CO₂(CO)₈) followed by reaction in the presence of a compound of formula XIX as hereinbefore defined, under reaction conditions known to those skilled in the art, for example such as those hereinbefore described in respect of preparation of compounds of formula I (process step (ii), e.g. (ii)(A)(b) above), e.g. the carbonylation step being performed in the presence of an appropriate precious metal (e.g. palladium) catalyst;
(IV) reaction of a compound of formula XXXVI,

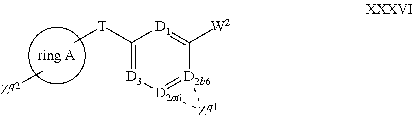

wherein W² represents a suitable group such as an appropriate alkali metal group (e.g. sodium, potassium or, especially, lithium), a —Mg-halide or a zinc-based group, and ring A, D₁, D₂ₐ₆, D_{2b6}, D₃, Z^{q1}, Z^{q2} and T are as hereinbefore defined, with e.g. CO₂ (in the case where R^{9a} in the compounds to be prepared represents hydrogen) or a compound of formula XVIII in which L^{xy} represents a single bond, Y^{b} represents —C(O)OR^{9a}, in which R^{9a} is other than hydrogen, and L⁶ represents a suitable leaving group, such as chloro or bromo or a C₁₋₁₄ (such as C₁₋₆ (e.g. C₁₋₃) alkoxy group), under reaction conditions known to those skilled in the art. The skilled person will appreciate that this reaction step may be performed directly after (i.e. in the same reaction pot) the preparation of compounds of formula XXXVI (which is described hereinafter).

Compounds of formula IX in which Z^{x} and Z^{y} represent a sulfonate group may be prepared from corresponding compounds in which the Z^{x} and Z^{y} groups represent a hydroxy group, with an appropriate reagent for the conversion of the hydroxy group to the sulfonate group (e.g. tosyl chloride, mesyl chloride, triflic anhydride and the like) under conditions known to those skilled in the art, for example in the presence of a suitable base and solvent (such as those described above in respect of process step (i), e.g. an aqueous solution of K₃PO₄ in toluene) preferably at or below room temperature (e.g. at about 10° C.).

Compounds of formulae XXIV and XXV may be prepared, for example, by reaction of a corresponding compound of formula XXVII or XXVI, respectively (all of which are as hereinbefore defined, e.g. in which L^{5b} represents bromo or, preferably, iodo), for example, in the presence of a nucleophile that is a source of cyano ions, e.g. potassium or, preferably, copper cyanide.

Compounds of formulae XXVI and XXVII in which L^{5b} represents a —Mg-halide may be prepared by reaction of a compound corresponding to a compound of formula XXVI or XXVII but in which L^{5b} represents a halo group (e.g. bromo or iodo), under standard Grignard formation conditions, for example in the presence of i-PrMgCl (or the like) in the presence of a polar aprotic solvent (such as THF) under inert reaction condition, and preferably at low temperature (such as at below 0° C., e.g. at about 30° C.). The skilled person will appreciate that these compounds may be prepared in situ (see e.g. the process for the preparation of compounds of formula I (process steps (xxii) and (xxiii)).

Compounds of formulae XXVIIIC or XXVIIID may be prepared by reaction of a corresponding compound of formula XXVI or XXVII, as hereinbefore defined (and preferably one in which L^{5b} is a —Mg-halide, such as —Mg—I), with dimethylformamide (or a similar reagent for the introduction of the aldehyde group), under standard Grignard reaction conditions known to those skilled in the art (for example those described herein).

Compounds of formulae XXXIV or XXXV in which T represents —CH₂— may be prepared by reduction of a corresponding compound of formulae XXXIV or XXXV in which T represents —C(O)— (or from compounds corresponding to compounds of formulae XXXIV or XXXV but in which T represents —CH(OH)—), for example under standard reaction conditions known to those skilled in the art, for example reduction in the presence of a suitable reducing reagent such as LiAlH₄, NaBH₄ or trialkylsilane (e.g. triethylsilane) or reduction by hydrogenation (e.g. in the presence of Pd/C).

Alternatively, compounds of formulae XXXIV or XXXV in which T represents —CH₂— may be prepared by reaction of a compound of formula XXXVII,

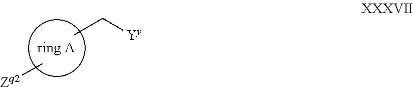

wherein Y^{y} represents a suitable group such as —OH, bromo, chloro or iodo, and ring A and Z^{q2} are as hereinbefore defined, with a compound of formula XXXVIII,

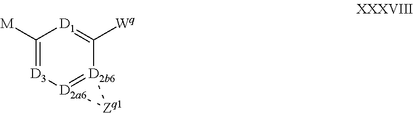

wherein M represents hydrogen and W^{q} represents hydrogen (for compounds of formula XXXIV) or W¹ (for compounds of formula XXXV) and D₁, D₂ₐ₆, D_{2b6}, D₃ and Z^{q1} are as hereinbefore defined, under standard conditions, for example in the presence of a Lewis or Brønsted acid. Alternatively, such compounds may be prepared from reaction of a compound of formula XXXVII in which Y^{y} represents bromo or chloro with a compound corresponding to a compound of formula XXXVIII but in which M represents —BF₃K (or the like), for example in accordance with the procedures described in Molander et al, J. Org. Chem. 71, 9198 (2006).

Compounds of formulae XXXIV or XXXV in which T represents —C(O)— may be prepared by reaction of a compound of formula XXXIX,

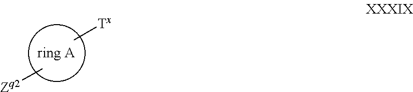

wherein T^{x} represents —C(O)Cl or —C═N—NH(t-butyl) (or the like) and ring A and Z^{q2} are as hereinbefore defined, with a compound of formula XXXVIII in which M represents hydrogen or an appropriate alkali metal group (e.g. sodium, potassium or, especially, lithium), a —Mg-halide or a zinc-based group, or, a bromo group, and D₁, D₂ₐ₆, D_{2b6}, D₃, Z^{q1} and W^{q} are as hereinbefore defined, under reaction conditions known to those skilled in the art. For example in the case of reaction of a compound of formula XXXIX in which T^{x} represents —C(O)Cl with a compound of formula XXXVIII in which M represents hydrogen, in the presence of an appropriate Lewis acid. In the case where M represents an appropriate alkali metal group, a —Mg-halide or a zinc-based group, under reaction conditions such as those hereinbefore described in respect of preparation of compounds of formulae III, IX, XXIX or XXXI (process step (IV) above) and preparation of compounds of formula XXXVI (see below). In the case of a reaction of a compound of formula XXXIX in which T^{x} represents —C═N—NH(t-butyl) (or the like) with a compound of formula XXXVIII in which M represents bromo, under reaction conditions such as those described in Takemiya et al, J. Am. Chem. Soc. 128, 14800 (2006).

For compounds corresponding to compounds of formula XXXIV or XXXV in which T represents —CH(OH)—, reaction of a compound corresponding to a compound of formula XXXIX, but in which T^{x} represents —C(O)H, with a compound of formula XXXVIII as defined above, under reaction conditions such as those hereinbefore described in respect of preparation of compounds of formulae XXXIV or XXXV in which T represents —C(O)—.

Compounds of formula XXXVI may be prepared in several ways. For example, compounds of formula XXXVI in which W² represents an alkali metal such as lithium, may be prepared from a corresponding compound of formula XXXIV (in particular those in which Z^{q1} and/or Z^{q2} represents a chloro or sulfonate group or, especially, a protected —NH₂ group, wherein the protecting group is preferably a lithiation-directing group, e.g. an amido group, such as a pivaloylamido group, or a sulfonamido group, such as an arylsulfonamido group, e.g. phenylsulfonamide), by reaction with an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. Alternatively, such compounds of formula XXXVI may be prepared by reaction of a compound of formula XXXV in which W¹ represents chloro, bromo or iodo by a halogen-lithium reaction in the presence of an organolithium base such as t- or n-butyllithium under reaction conditions such as those described above. Compounds of formula XXXVI in which W² represents —Mg-halide may be prepared from a corresponding compound of formula XXXV in which W¹ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XXXVI in which W² represents a zinc-based group (e.g. using ZnCl₂).

Compounds mentioned herein (e.g. those of formulae IV, V, VA, VI, VII, X, XI, XII, XIII, XIV, XV, XVI, XVII, XVIII, XIX, XX, XXI, XXII, XXIII, XXIV, XXV, XXVI, XXVII, XXVIII, XXVIIIB, XXIX, XXX, XXXII, XXXIII, XXXVII, XXXVIII and XXXIX) are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, the compounds described herein may also be prepared in accordance with synthetic routes and techniques described in international patent application WO 2006/077366.

The substituents D₁, D₂ₐ, D_{2b}, D₃, L¹, Y¹, L², Y², L³ and Y³ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications (e.g. from a carboxylic acid, e.g. in the presence of H₂SO₄ and appropriate alcohol or in the presence of K₂CO₃ and alkyl iodide), etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where Y¹ (or, if present, Y^{1a}) represents —C(O)OR^{9a} in which R^{9a} does not initially represent hydrogen (so providing at least one ester functional group), the skilled person will appreciate that at any stage during the synthesis (e.g. the final step), the relevant R^{9a}-containing group may be hydrolysed to form a carboxylic acid functional group (i.e. a group in which R^{9a} represents hydrogen). In this respect, the skilled person may also refer to “Comprehensive Organic Functional Group Transformations” by A. R. Katritzky, O. Meth-Cohn and C. W. Rees, Pergamon Press, 1995. Other specific transformation steps include the reduction of a nitro group to an amino group, the hydrolysis of a nitrile group to a carboxylic acid group, and standard nucleophilic aromatic substitution reactions, for example in which an iodo-, preferably, fluoro- or bromo-phenyl group is converted into a cyanophenyl group by employing a source of cyanide ions (e.g. by reaction with a compound which is a source of cyano anions, e.g. sodium, copper (I), zinc or, preferably, potassium cyanide) as a reagent (alternatively, in this case, palladium catalysed cyanation reaction conditions may also be employed).

Other transformations that may be mentioned include: the conversion of a halo group (preferably iodo or bromo) to a 1-alkynyl group (e.g. by reaction with a 1-alkyne), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate reagent/nucleophile e.g. a source of the relevant reagent/anion, for example by reaction in the presence of a reagent that is a source of halogen (e.g. CuCl, CuBr or NaI), or a reagent that is a source of azido or cyanide anions, such as NaN₃, CuCN or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Further, the skilled person will appreciate that the D₁ to D₃-containing ring, as well as the A ring may be heterocycles, which moieties may be prepared with reference to a standard heterocyclic chemistry textbook (e.g. “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 or “Science of Synthesis”, Volumes 9-17 (Hetarenes and Related Ring Systems), Georg Thieme Verlag, 2006). Hence, the reactions disclosed herein that relate to compounds containing hetereocycles may also be performed with compounds that are pre-cursors to heterocycles, and which pre-cursors may be converted to those heterocycles at a later stage in the synthesis.

Compounds of the invention may be isolated (or purified) from their reaction mixtures using conventional techniques (e.g. crystallisations, recrystallisations or chromatographic techniques).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein. Protecting groups that may be mentioned include lactone protecting groups (or derivatives thereof), which may serve to protect both a hydroxy group and an α-carboxy group (i.e. such that the cyclic moiety is formed between the two functional groups.

The type of chemistry involved will dictate the need, and type, of protecting groups as well as the sequence for accomplishing the synthesis.

The use of protecting groups is described in e.g. “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined, for use as a pharmaceutical and/or in isolated (i.e. ex vivo) form.

Hence, in an embodiment of the invention, there is provided a compound of the invention as hereinbefore defined, in an isolated (or ex vivo) form. Such forms are particularly preferred for compounds of formula I in which: (i) L² and, optionally, L³ represents —S(O)—; and/or (ii) when L² represents —S(O)ᵣ— (in which r is preferably 1) and L³ represents —S(O)ᵣ— (in which r is preferably 1, i.e. —S(O)—).

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention. Preferably, for compounds of the invention in which L² and/or L³ represent(s) —S(O)—, the term “prodrug” preferably does not include corresponding compounds of the invention in which either L² and/or L³ represent(s) —S— or —S(O)₂—.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention, including, but not limited to:
(a) compounds of formula I in which Y¹ (or, if present, Y^{1a}) represents —C(O)OR^{9a} in which R^{9a} is/are other than hydrogen, so forming an ester group; and/or
(b) compounds of formula I in which Y represents —C(═N—OR²⁸)—, i.e. the following compound of formula Ia,

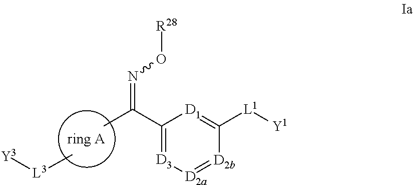

in which the integers are as hereinbefore defined (and the squiggly line indicates that the oxime may exist as a cis or trans isomer, as is apparent to the skilled person),
   may possess no or minimal pharmacological activity as such, but may be administered parenterally or orally, and thereafter be metabolised in the body to form compounds of the invention that possess pharmacological activity as such, including, but not limited to:
(A) corresponding compounds of formula I, in which Y¹ (or, if present, Y^{1a}) represents —C(O)OR^{9a} in which R^{9a} represent hydrogen (see (a) above); and/or
(B) corresponding compounds of formula I in which Y represents —C(O)—, for example in the case where the oxime or oxime ether of the compound of formula Ia (see (b) above) is hydrolysed to the corresponding carbonyl moiety.

Further compounds of the invention that may be metabolised include those in which there is a —S(O)— moiety present, which may be converted (by appropriate oxidation or reduction in vivo), to a corresponding compound in which there is either a —S— or —S(O)₂— moiety present.

Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III (preferably, MGST-II), thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTC₄ and/or LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. leukotriene receptor antagonists (LTRas), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activting protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined; and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of a respiratory disorder and/or inflammation, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Aqueous solubility is a fundamental molecular property that governs a large range of physical phenomena related to the specific chemical compound including e.g. environmental fate, human intestinal absorption, effectiveness of in vitro screening assays, and product qualities of water-soluble chemicals. By definition, the solubility of a compound is the maximum quantity of compound that can dissolve in a certain quantity of solvent at a specified temperature. Knowledge of a compound's aqueous solubility can lead to an understanding of its pharmacokinetics, as well as an appropriate means of formulation.

Compounds of the invention may exhibit improved solubility properties. Greater aqueous solubility (or greater aqueous thermodynamic solubility) may have advantages related to the effectiveness of the compounds of the invention, for instance improved absorption in vivo (e.g. in the human intestine) or the compounds may have other advantages associated with the physical phenomena related to improved aqueous stability (see above). Good (e.g. improved) aqueous solubility may aid the formulation of compounds of the invention, i.e. it may be easier and/or less expensive to manufacture tablets which will dissolve more readily in the stomach as potentially one can avoid esoteric and/or expensive additives and be less dependent on particle-size (e.g. micronization or grinding may be avoided) of the crystals, etc, and it may be easier to prepare formulations intended for intravenous administration.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

### In Vitro Assay

In the assay, LTC₄ synthase catalyses the reaction where the substrate LTA₄ is converted to LTC₄. Recombinant human LTC₄ synthase is expressed in Piccia pastoralis and the purified enzyme is dissolved in 25 mM tris-buffer pH 7.8 supplemented with 0.1 mM glutathione (GSH) and stored at −80° C. The assay is performed in phosphate buffered saline (PBS) pH 7.4 and 5 mM GSH in 384-well plates.

The following is added chronologically to each well:

1. 48 μL LTC₄ synthase in PBS with 5 mM GSH. The total protein concentration in this solution is 0.5 μg/mL.
2. 1 μL inhibitor in DMSO (final concentration 10 μM).
3. Incubation of the plate at room temperature for 10 min.
4. 1 μL LTA₄ (final concentration 2.5 μM).
5. Incubation of the plate at room temperature for 5 min.
6. 10 μL of the incubation mixture is analysed using homogenous time resolved fluorescent (HTRF) detection.

### EXAMPLES

In the event that there is a discrepancy between nomenclature and any compounds depicted graphically, then it is the latter that presides (unless contradicted by any experimental details that may be given or unless it is clear from the context).

The invention is illustrated by way of the following examples, in which the following abbreviations may be employed:

| | |
|---|---|
| aq | aqueous |
| BINAP | 2,2′-bis(diphenylphosphino)-1,1′-binaphthyl |
| brine | saturated aqueous solution of NaCl |
| DMF | dimethylformamide |
| EtOAc | ethyl acetate |
| MeCN | acetonitrile |
| MeOH | methanol |
| NMR | nuclear magnetic resonance |
| Oxone | potassium peroxymonosulfate (2KHSO₅•KHSO₄•K₂SO₄) |
| rt | room temperature |
| rx | reflux |
| sat | saturated |
| THF | tetrahydrofuran |
| TLC | thin layer chromatography |
| xantphos | 4,5-bis(diphenylphosphino)-9,9-dimethylxanthene |

Chemicals specified in the synthesis of the compounds in the examples were commercially available from, e.g. Sigma-Aldrich Fine Chemicals or Acros Int.

### Example 1:1

### 5-{4-[(4-Chlorophenyl)(methyl)amino]benzoyl}-2-(1-hexylsulfinyl)benzoic acid

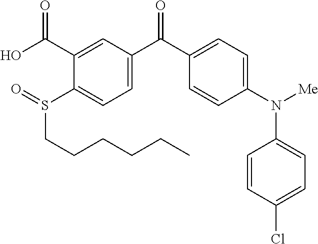

### (a) 5-(4-Bromobenzoyl)-2-fluorobenzoic acid methyl ester

i-PrMgCl.LiCl in THF (1.1 M, 4.63 μL, 5.0 mmol) was added to 2-fluoro-5-iodo-benzoic acid methyl ester (1.02 g, 3.64 mmol) in THF (15 mL) at −30° C. After 2 h at that temperature, the mixture was cooled to −65° C. and 4-bromobenzoyl chloride (1.75 g, 8.02 mmol) in THF (10 mL) was added. The mixture was stirred at −65° C. for 1 h and at 5° C. overnight. NH₄Cl (aq, sat) was added. Extractive workup (EtOAc, H₂O, brine) and purification by chromatography gave the sub-title compound. Yield: 0.58 g (48%).

### (b) 5-{4-[4-Chlorophenyl)amino]benzoyl}-2-fluorobenzoic acid methyl ester

A mixture of 5-(4-bromobenzoyl)-2-fluorobenzoic acid methyl ester (0.52 g, 1.54 mmol), 4-chloro-N-methylaniline (0.26 g, 1.85 mmol), Pd(OAc)₂ (35 mg, 0.16 mmol), BINAP (50 mg, 0.155 mmol), Cs₂CO₃ (1.5 g, 4.6 mmol) and toluene (10 mL) was heated at 80° C. for 16 h. The mixture was diluted with EtOAc and filtered through Celite. The combined filtrates were concentrated and the residue purified by chromatography to give the sub-title compound. Yield: 0.30 g (49%).

### (c) 5-{4-[(4-Chlorophenyl)(methyl)amino]benzoyl}-2-(1-hexylsulfanyl)benzoic acid methyl ester

A mixture of 5-{5-[(4-chlorophenyl)amino]benzoyl}-2-fluorobenzoic acid methyl ester (0.3 g, 0.75 mmol), 1-hexanethiol (98.7 mg, 0.83 mmol), KF/Al₂O₃ (0.2 g), 18-crown-6 (16 mg, 0.06 mmol) and MeCN (15 mL) was heated at rx for 16 h. The mixture was diluted with EtOAc, followed by extractive workup (EtOAc, H₂O, brine) and purification by chromatography to give the sub-title compound. Yield: 0.29 g (78%).

### (d) 5-{4-[(4-Chlorophenyl)(methyl)amino]benzoyl}-2-(1-hexylsulfinyl)benzoic acid methyl ester

Oxone (0.56 g, 0.9 mmol) in H₂O (5 mL) was added to 5-{5-[(4-chlorophenyl)-(methyl)amino]benzoyl}-2-(1-hexylsulfanyl)benzoic acid methyl ester (0.14 g, 0.28 mmol) in THF (5 mL) at 0° C. The mixture was stirred at 0° C. for 15 min and at rt overnight. Extractive workup (EtOAc, H₂O, brine) and purification by chromatography gave the sub-title compound. Yield: 100 mg (70%).

### (e) 5-{4-[(4-Chlorophenyl)(methyl)amino]benzoyl}-2-(1-hexylsulfinyl)benzoic acid

A mixture of 5-{5-[(4-chlorophenyl)(methyl)amino]benzoyl}-2-(1-hexylsulfinyl)-benzoic acid methyl ester, NaOH (2 M, 2 mL) and dioxane (5 mL) was stirred at rt for 30 min. Neutralisation with HCl (2 M), extractive workup (EtOAc, H₂O, brine), drying (Na₂SO₄) and concentration gave the title product. Yield: 85 mg (85%). ¹H NMR (DMSO-d₆) δ: 8.36-8.00 (3H, m) 7.81-7.59 (2H, m) 7.58-7.41 (2H, m) 7.40-7.18 (2H, m) 6.96-6.71 (2H, m) 3.3 (3H, s) 3.2-3.0 (1H, m) 2.7-2.5 (1H, m) 1.89-1.66 (1H, m) 1.62-1.12 (7H, m) 0.93-0.72 (3H, m).

### Examples 1:2-1:10

The title compounds were synthesized from 5-{4-[(4-chlorophenyl)amino]-benzoyl}-2-fluorobenzoic acid methyl ester (see Example 1:1, step (b)) and the appropriate thiol, followed by oxidation and hydrolysis in accordance with Example 1:1, steps (c), (d) and (e), see Table 1.

### Example 1:11

### 5-{4-[(4-Chlorophenyl)(cyclopropylmethyl)amino]benzoyl}-2-phenylsulfinylbenzoic acid

### (a) 5-[4-(4-Chlorophenylaminobenzoyl]-2-fluoro-benzoic acid methyl ester

The sub-title compound was prepared from 5-(4-bromobenzoyl)-2-fluorobenzoic acid methyl ester and 4-chloroaniline in accordance with Example 1:1, step (b). Yield: 82%.

### (b) 5-{4-([4-Chlorophenyncyclopropylmethylamino]benzoyl}-2-fluorobenzoic acid methyl ester

A mixture of 5-[4-(4-chlorophenylamino)benzoyl]-2-fluorobenzoic acid methyl ester (2.86 g, 7.71 mmol), cyclopropylmethylbromide (5.12 g, 23.13 mmol) and DMF (58 mL) was cooled to 0° C. Sodium hydride (60% in mineral oil, 0.329 g, 8.25 mmol) was added and the mixture stirred at rt until full conversion was achieved as judged by TLC. Extractive workup (EtOAc, water, brine), drying (Na₂SO₄) and purification by chromatography gave the sub-title compound. Yield: 2.32 g (69%).

### (c) 5-{4-[(4-Chlorophenyl)(cyclopropylmethyl)amino]benzoyl}-2-phenylsulfinyl-benzoic acid

The title compound was prepared from 5-{4-[(4-chlorophenyl)cyclopropylmethyl-amino]benzoyl}-2-fluorobenzoic acid methyl ester and thiophenol followed by oxidation and hydrolysis in accordance with Example 1:1, steps (c), (d) and (e), see Table 1.

### Example 1:12

### 5-{4-[(4-Chlorophenyl)cyclopropylmethylamino]benzoyl}-2-(1-hexylsulfinyl)-20 benzoic acid

The title compound was prepared in accordance with Example 1:11 from 5-{4-[(4-chlorophenyl)cyclopropylmethylamino]benzoyl}-2-fluorobenzoic acid methyl ester and 1-hexanethiol, see Table 1.

### Example 1:13

### 5-{5-[(4-Chlorophenyl)methylamino]pyridine-2-carbonyl]-2-(3,4-difluorobenzene-sulfinyl)benzoic acid

### (a) 2-Fluoro-5-formylbenzoic acid methyl ester

i-PrMgCl.LiCl complex in THF (1.0 M, 70 mL, 70.0 mmol) was added to 2-fluoro-5-iodobenzoic acid methyl ester (13.0 g, 46.4 mmol) in THF (80 mL) at −45° C. After stirring at −40° C. for 1 h, DMF (2.7 mL, 35.7 mmol) was added. The temperature was allowed to reach rt over 1 h and HCl (1 M, aq, 300 mL) was added. Extractive workup (EtOAc, water, brine) and concentration gave the sub-title product. Yield: 8.95 g (98%).

### (b) 5-[(5-Bromo-2-pyridyl)hydroxymethyl]-2-fluorobenzoic acid methyl ester

i-PrMgCl in THF (2.0M, 24 mL, 48.9 mmol) was added to 5-bromo-2-iodopyridine (13.2 g, 46.6 mmol) in THF (50 mL) at −15° C. After stirring at −15° C. for 1 h, 2-fluoro-5-formylbenzoic acid methyl ester (8.50 g, 48.9 mmol) in THF (50 mL) was added at −45° C. The mixture was stirred at rt for 6 h and quenched with NH₄Cl (aq, sat). Extractive workup (EtOAc, water, brine) and purification by chromatography gave the sub-title compound. Yield: 13.4 g (85%).

### (c) 5-(5-Bromopyridine-2-carbonyl)-2-fluoro-benzoic acid methyl ester

Pyridinium chlorochromate (8.94 g, 41.5 mmol) was added to 5-[(5-bromo-2-pyridinyl)hydroxymethyl]-2-fluorobenzoic acid methyl ester (13.4 g, 39.5 mmol) in CH₂Cl₂ (400 mL) at rt. After 1 h the mixture was filtered though Celite and concentrated. The residue was treated with EtOAc and hexane (1:2) and filtered through silica gel. Concentration of the combined filtrates gave the sub-title compound. Yield: 10.7 g (80%).

### (d) 5-{5-[(4-Chlorophenyl)methylamino]pyridine-2-carbonyl}-2-fluorobenzoic acid methyl ester

The sub-title compound was prepared from 5-(5-bromopyridine-2-carbonyl)-2-fluorobenzoic acid methyl ester and 4-chloro-N-methylaniline in accordance with Example 1:1, step (b).

### (e) 5-{5-[(4-Chlorophenyl)methylamino]pyridine-2-carbonyl}-2-(3,4-difluoro-benzenesulfinyl)benzoic acid

The title compound was prepared from 5-{5-[(4-chlorophenyl)methylamino]-pyridine-2-carbonyl}-2-fluorobenzoic acid methyl ester and 3,4-difluorothiophenol followed by oxidation and hydrolysis in accordance with Example 1:1, steps (c), (d) and (e), see Table 1.

### Example 1:14

### 5-{5-[(4-Chlorophenyl)cyclopropylmethylamino]pyridine-2-carbonyl}-2-(4-trifluoro-methylphenylsulfinyl)benzoic acid

### (a) 5-{5-[(4-Chlorophenyl)cyclopropylmethylamino]pyridine-2-carbonyl}-2-fluorobenzoic acid methyl ester

The sub-title compound was prepared from 5-(5-bromopyridine-2-carbonyl)-2-fluorobenzoic acid methyl ester (see Example 1:13, step (d)) and 4-chloro-N-cyclopropylmethylaniline in accordance with Example 1:1, step (b).

### (b) 5-{5-[(4-Chlorophenyl)cyclopropylmethylamino]pyridine-2-carbonyl}-2-(4-trifluoromethylphenylsulfinyl)benzoic acid

The title compound was prepared from 5-{5-[(4-chlorophenyl)cyclopropylmethyl-amino]pyridine-2-carbonyl}-2-fluorobenzoic acid methyl ester and the appropriate thiol, followed by oxidation and hydrolysis in accordance with Example 1:1, steps (c), (d) and (e), see Table 1.

### Examples 1:15-1:18

The title compounds were prepared in accordance with Example 1:14, using the appropriate thiol, see Table 1.

### Example 1:19

### 3-{4-[(4-Chlorophenyl)(methyl)amino]benzoyl}-5-(3,4-difluorophenylsulfinyl)-benzoic acid

### (a) 3-Bromo-5-iodobenzoic acid methyl ester

Na₂CO₃ (9.7 g, 92 mmol) and MeI (5.7 mL, 92 mmol) was added to a mixture of 3-bromo-5-iodobenzoic acid (15 g, 45.9 mmol), THF (20 mL) and DMF (75 mL). The mixture was stirred at rt for 20 h and concentrated. Extractive workup (EtOAc, H₂O, NaHCO₃ (aq, sat), brine) and concentration gave the sub-title compound. Yield: 15 g (99%).

### (b) 4-[(4-Chlorophenyl)(methyl)amino]benzaldehyde

Toluene (100 mL), followed by 4-chloro-N-methylaniline (4.58 mL, 37.8 mmol) were added to a mixture of Cs₂CO₃ (17.26 g, 53 mmol), Pd(OAc)₂ (0.42 g, 1.9 mmol), BINAP (1.77 g, 2.8 mmol) and 4-bromobenzaldehyde (7 g, 37.8 mmol). The mixture was stirred at 85° C. for 20 h and filtered through Celite. The solids were washed with EtOAc. The combined filtrates were concentrated and the residue purified by chromatography to give the sub-title compound. Yield: 7.7 g (82%).

### (c) 3-Bromo-5-[4-(4-chlorophenyl(methyl)amino)benzoyl]benzoic acid methyl ester

i-PrMgCl in THF (22.5 mL, 29 mmol, 1.3 M) was added dropwise to a mixture of 3-bromo-5-iodobenzoic acid methyl ester (8.54 g, 25 mmol) and THF (150 mL) at −15° C. The mixture was stirred at −15° C. for 80 min and cooled to −45° C. 4-[(4-Chlorophenyl)(methyl)amino]benzaldehyde (4.3 g, 17.5 mmol) in THF (30 mL) was added dropwise and the mixture was stirred for 20 min at −45° C. and at rt for 20 h. NH₄Cl (aq, sat) was added. Extractive workup (EtOAc, H₂O, brine) and concentration gave a residue (10 g).

A mixture of the residue (8 g, 17.4 mmol), DMF (150 mL) and MnO₂ (32 g, 368 mmol) was stirred at rt for 24 h. Filtration, concentration, crystallization from EtOAc, washing with isohexane and drying gave the sub-title compound. Yield: 6 g (75%).

### (d) 3-{4-[(4-Chlorophenyl-methylamino]benzoyl}-5-(3,4-difluorophenyl-sulfanyl)benzoic acid methyl ester

A mixture of 3-bromo-5-[4-(4-chlorophenyl(methyl)amino)benzoyl]benzoic acid methyl ester (0.20 g, 0.44 mmol), i-PrNEt₂ (113 mg, 0.88 mmol), dioxane (2 mL), Pd₂(dba)₃ (10.1 mg, 0.011 mmol), xantphos (12.7 mg, 0.022 mmol) and 3,4-di-fluorothiophenol (64 mg, 0.44 mmol) was heated at reflux for 20 h. The mixture was filtered through Celite and the solids washed with EtOAc. Concentration of the combined filtrates and purification of the residue by chromatography gave the sub-title compound. Yield: 152 mg (67%).

### (e) 5-{4-[(4-Chlorophenyl)(methyl)amino]benzoyl}-3-(3,4-difluororophenyl-sulfinyl)benzoic acid methyl ester

Oxone (528 mg, 0.86 mmol) in H₂O (7 mL) was added to 3-{-[(4(4-chlorophenyl)-methylamino]benzoyl}-5-(3,4-difluorophenylsulfanyl)benzoic acid methyl ester (150 mg 0.29 mmol) in THF (7 mL) at 0° C. The mixture was stirred at 0° C. for 10 min and at rt for 5 h. Extractive workup (EtOAc, H₂O, brine) and purification by chromatography gave the sub-title compound. Yield: 100 mg (65%).

### (f) 5-{4-[(4-Chlorophenyl)(methyl)amino]benzoyl}-3-(3,4-difluororophenyl-sulfinyl)benzoic acid

A mixture of 5-{4-[(4-chlorophenyl)(methyl)amino]benzoyl}-3-(3,4-difluororophenyl-sulfinyl)benzoic acid methyl ester (170 mg, 0.33 mmol), NaOH (1 M, aq, 16.4 mL, 1.64 mmol) and EtOH (50 mL) was stirred at 70° C. for 40 min. Acidification with 1 M HCl, extractive workup (EtOAc, H₂O, brine), drying (Na₂SO₄) and concen-tration gave the title product. Yield: 70 mg (42%). See Table 1.

### Example 1:20

### 3-{5-[(4-Chlorophenyl)methylamino]pyridine-2-carbonyl}-5-(1-hexylsulfinyl)-benzoic acid

### (a) 5-[(4-Chlorophenyl)methylamino]pyridine-2-carbaldehyde

The sub-title compound was prepared from 5-bromo-2-formylpyridine and 4-chloro-N-methylaniline in accordance with Example 1:1, step (b). Yield 2.5 g (96%).

### (b) 3-{5-[(4-Chlorophenyl)methylamino]pyridine-2-carbonyl}-5-iodobenzoic acid methyl ester

The sub-title compound was prepared from 3,5-diiodobenzoic acid methyl ester and 5-[(4-chlorophenyl)methylamino]pyridine-2-carbaldehyde in accordance with Example 1:19, step (c), (Yield: 40%) followed by oxidation in accordance with Example 1:13, step (c). Yield: (50%).

### (c) 3-{5-[(4-Chlorophenyl)methylamino]pyridine-2-carbonyl}-5-(1-hexyl-sulfanyl)benzoic acid methyl ester

A mixture of 3-{5-[(4-chlorophenyl)methylamino]pyridine-2-carbonyl}-5-iodo-benzoic acid methyl ester (0.285 g, 0.56 mmol), 1-hexanethiol (0.072 g, 0.61 mmol), Pd₂(dba)₃ (0.018 g, 0.02 mmol), bis(2-diphenylphosphinophenyl)ether (0.018 g, 0.034 mmol), potassium tert-butoxide (0.126 g, 1.12 mmol) and toluene (10 mL) was stirred at Tx for 6 h. Filtration through Celite, washing with EtOAc, concentration of the combined filtrates and purification of the residue by chromatography gave the sub-title compound. Yield 0.08 g (28%).

### (d) 3-{5-[(4-Chlorophenyl)methylamino]pyridine-2-carbonyl}-5-(1-hexyl-sulfinyl)benzoic acid

The title compound was prepared from 3-{5-[(4-chlorophenyl)methylamino]-pyridine-2-carbonyl}-5-(1-hexylsulfanyl)benzoic acid methyl ester by oxidation and hydrolysis in accordance with Example 1:19, steps (e) and (f), see Table 1.

### Example 1:21

### 5-(5-((4-Chlorophenyl)(methyl)amino)pyrimidine-2-carbonyl)-2-(1-hexylsulfinyl)-benzoic acid

### (a) 5-(5-Bromopyrimidine-2-carbonyl)-2-fluorobenzoic acid methyl ester

The sub-title compound was prepared from 2-fluoro-5-iodobenzoic acid methyl ester and 5-bromo-2-cyanopyrimidine in accordance with Example 1:1, step (a).

### (b) 5-(5-((4-Chlorophenyl)(methyl)amino)pyrimidine-2-carbonyl)-2-(1-hexyl-sulfinyl)benzoic acid

The title compound was synthesized from 5-(5-bromopyrimidine-2-carbonyl)-2-fluorobenzoic acid methyl ester in accordance with Example 1:1, steps (b), (c), (d) and (e), see Table 1.

### Example 1:22

### 5-{5-[(4-Chlorophenyl)methylamino]pyridine-2-carbonyl}-2-(1-hexylsulfinyl)-benzoic acid

The title compound was prepared in accordance with Example 1:13 from 5-{5-[(4-chlorophenyl)methylamino]pyridine-2-carbonyl}-2-fluorobenzoic acid methyl ester (see Example 1:13, step (d)) and 1-hexanethiol, followed by oxidation and hydrolysis in accordance with Example 1:1, steps (c), (d) and (e), see Table 1.

TABLE 1

| | Chemical structure |
|---|---|
| | Name |
| Ex. | ¹H-NMR (DMSO-d₆, δ) |
| 1:2 | 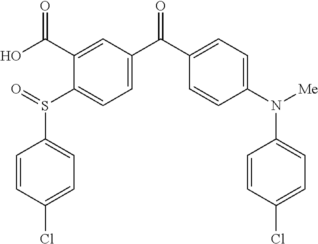 |
| | 5-{4-[(4-Chlorophenyl)(methyl)amino]benzoyl}-2-(4-chlorophenylsulfinyl)benzoic |
| | acid |
| | 8.44 (1H, d, J = 8.5 Hz) 8.24-8.15 (2H, m) 7.77-7.63 (4H, m) 7.63-6.48 (4H, m) |
| | 7.40-7.30 (2H, m) 6.94-6.82 (2H, m) 3.40 (3H, s) |
| 1:3 | 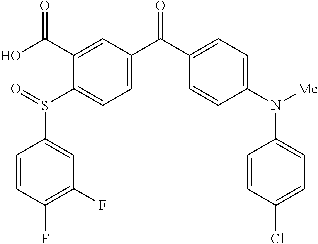 |
| | 5-{4-[(4-Chlorophenyl)(methyl)amino]benzoyl}2-(3,4-difluorophenylsulfinyl)- |
| | benzoic acid |
| | 8.44 (1H, d, J = 8.2 Hz) 8.27-8.13 (2H, m) 7.88-7.47 (7H, m) 7.43-7.29 (2H, m) |
| | 6.95-6.81 (2H, m) 3.3 (3H, s) |
| 1:4 | 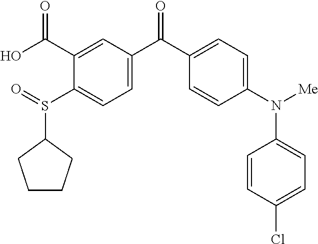 |
| | 5-{4-[(4-Chlorophenyl)(methyl)amino]benzoyl}-2-(cyclopentylsulfinyl)benzoic |
| | acid |
| | 8.25 (1H, d, J = 1.3 Hz) 8.17-8.06 (2H, m) 7.74-7.65 (2H, m) 7.57-7.48 (2H, m) |
| | 7.40-7.31 (2H, m) 6.94-6.83 (2H, m) 3.58-3.48 (1H, m) 3.38 (3H, s) 2.18-2.07 |
| | (1H, m) 2.02-1.91 (1 H, m) 1.88-1.76 (1 H, m) 1.69-1.54 (3H, m) 1.52-1.41 (1H, |
| | m) 1.17-1.05 (1H, m) |
| 1:5 | 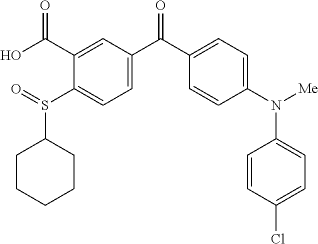 |
| | 5-{4-[(4-Chlorophenyl)(methyl)amino]benzoyl}-2-(cyclohexylsulfinyl)benzoic acid |
| | 8.33-8.22 (1H, m) 8.17-8.02 (2H, m) 7.76-7.63 (2H, m) 7.59-7.46 (2H, m) 7.42- |
| | 7.28 (2H, m) 6.98-6.81 (2H, m) 3.38 (3H, m) 3.05-2.94 (1 H, m) 2.21-2.11 (1 H, |
| | m) 1.91-1.81 (1H, m) 1.77-1.54 (3H, m) 1.50-1.25 (2H, m) 1.23-1.04 (3H, m) |
| 1:6 | 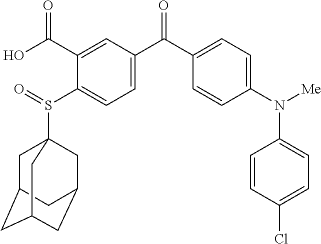 |
| | 2-(1-Adamantylsulfinyl)-5-{4-[(4-chlorophenyl)methylamino]benzoyl}benzoic |
| | acid |
| | 8.27-8.17 (1H, m) 8.13-7.95 (2H, m) 7.77-7.61 (2H, m) 7.60-7.44 (2H, m) 7.43- |
| | 7.25 (2H, m) 7.00-6.78 (2H, m) 3.38 (3H, s) 2.14-1.97 (3H, m) 1.78-1.45 (12H, m) |
| 1:7 | 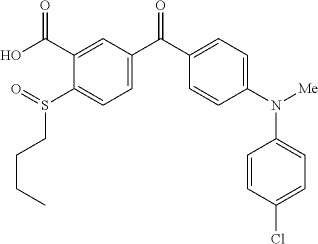 |
| | 2-(1-Butylsulfinyl)-5-{4-[(4-chlorophenyl)(methyl)amino]benzoyl}benzoic acid |
| | 8.24 (1H, d, J = 1.5 Hz) 8.19 (1H, d, J = 8.1 Hz) 8.09 (1H, dd, J = 8.1; 1.5 Hz) 7.75- |
| | 7.63 (2H, m) 7.58-7.45 (2H, m) 7.41-7.29 (2H, m) 6.96-6.84 (2H, m) 3.38 (3H, s) |
| | 3.27-3.17 (1H, m) 2.71-2.61 (1H, m) 1.85-1.75 (1H, m) 1.59-1.33 (3H, m) 0.96- |
| | 0.85 (3H, t, J = 7.1 Hz) |
| 1:8 | 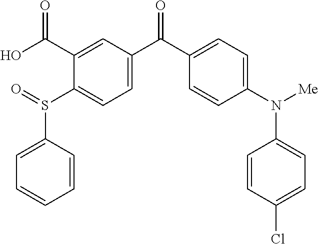 |
| | 5-{4-[(4-Chlorophenyl)(methyl)amino]benzoyl}-2-(phenylsulfinyl)benzoic acid |
| | 8.39 (1H, d, J = 8.1 Hz) 8.17 (1H, d, J = 1.5 Hz) 8.14-8.06 (1H, m) 7.78-7.70 (2H, |
| | m) 7.70-7.64 (2H, m) 7.57-7.41 (5H, m) 7.40-7.29 (2H, m) 6.95-6.83 (2H, m) |
| | 3.37 (3H, s) |
| 1:9 | 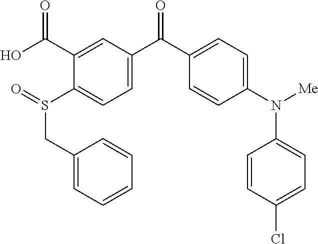 |
| | 2-(Benzylsulfinyl)-5-{4-[(4-chlorophenyl)(methyl)amino]benzoyl}benzoic acid |
| | 8.26 (1H, d, J = 1.6 Hz) 7.96 (1H, dd, J = 8.1; 1.6 Hz) 7.88 (1H, d, J = 8.1 Hz) 7.75- |
| | 7.61 (2H, m) 7.59-7.44 (2H, m) 7.43-7.24 (5H, m) 7.24-7.11 (2H, m) 6.98-6.83 |
| | (2H, m) 4.54 (1H, d, J = 12.7 Hz) 3.90 (1H, d, J = 12.7 Hz) 3.39 (3H, s) |
| 1:10 | 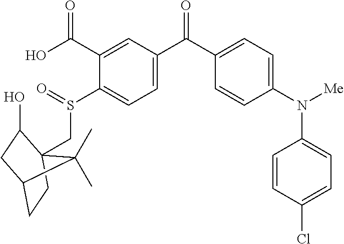 |
| | 5-{4-[(4-Chlorophenyl)(methyl)amino]benzoyl}-2-[(2-hydroxy-7,7-dimethyl- |
| | bicyclo[2.2.1]-1-heptyl)methylsulfinyl]benzoic acid |
| | 8.31-8.26 (1H, m) 8.24 (1H, d, J = 1.6 Hz) 8.12 (1H, dd, J = 8.3; 1.6 Hz) 7.73-7.66 |
| | (2H, m) 7.55-7.46 (2H, m) 7.38-7.30 (2H, m) 6.91-6.85 (2H, m) 4.37 (1H, s) |
| | 4.06-3.98 (1H, m) 3.36 (3H, s) 3.22-3.16 (1H, m) 2.95-2.88 (1H, m) 2.00-1.90 |
| | (1H, m) 1.80-1.65 (4H, m) 1.58-1.49 (1H, m) 1.19-1.12 (1H, m) 0.98 (3H, s) 0.79 |
| | (3H, s) |
| 1:11 | 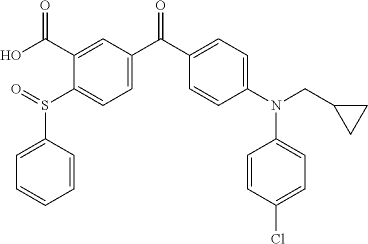 |
| | 5-{4-[(4-Chlorophenyl)(cyclopropylmethyl)amino]benzoyl}-2-phenylsulfinyl- |
| | benzoic acid |
| | 8.38 (1H, d, J = 8.2 Hz) 8.15 (1H, d, J = 1.6 Hz) 8.09 (1H, dd, J = 8.2; 1.6 Hz) 7.75- |
| | 7.67 (2H, m) 7.67-7.59 (2H, m) 7.55-7.48 (2H, m) 7.48-7.40 (3H, m) 7.36-7.28 |
| | (2H, m) 6.85-6.75 (2H, m) 3.65 (2H, d, J = 6.6 Hz) 1.13-1.03 (1H, m) 0.48-0.38 |
| | (2H, m) 0.17-0.07 (2H, m) |
| 1:12 | 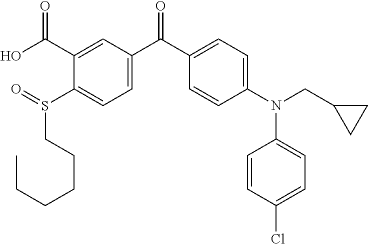 |
| | 5-{4-[(4-Chlorophenyl)cyclopropylmethylamino]benzoyl}-2-(1-hexylsulfinyl)- |
| | benzoic acid |
| | 8.21 (1H, d, J = 1.6 Hz) 8.16 (1H, d, J = 8.2 Hz) 8.05 (1H, dd, J = 8.2; 1.6 Hz) 7.70- |
| | 7.60 (2H, m) 7.57-7.47 (2H, m) 7.38-7.28 (2H, m) 6.87-6.77 (2H, m) 3.66 (2H, d, |
| | J = 6.6 Hz) 3.25-3.15 (1 H, m) 2.69-2.59 (1H, m) 1.85-1.72 (1H, m) 1.60-1.47 (1H, |
| | m) 1.47-1.31 (2H, m) 1.31-1.17 (4H, m) 1.14-1.03 (1H, m) 0.92-0.75 (3H, m) |
| | 0.50-0.36 (2H, m) 0.19-0.06 (2H, m) |
| 1:13 | 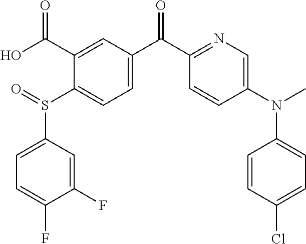 |
| | 5-{5-[(4-Chlorophenyl)methylamino]pyridine-2-carbonyl}-2-(3,4-difluorophenyl- |
| | sulfinyl)benzoic acid |
| | 8.41-8.34 (1H, m) 8.19-8.08 (3H, m) 7.99-7.92 (2H, m) 7.76-7.69 (1H, m) 7.55- |
| | 7.45 (3H, m) 7.40-7.35 (2H, m) 7.29 (1 H, dd, J = 9.0; 2.8 Hz) 3.39 (3H, s) |
| 1:14 | 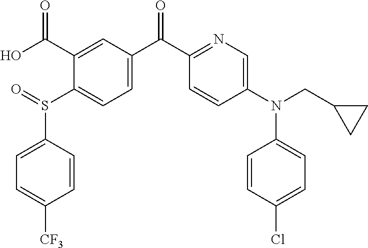 |
| | 5-{5-[(4-Chlorophenyl)cyclopropylmethylamino]pyridine-2-carbonyl}-2-(4-tri- |
| | fluoromethylphenylsulfinyl)benzoic acid |
| | 8.38 (1 H, d, J = 1.7 Hz) 8.32 (1 H, dd, J = 8.2; 1.7 Hz) 8.25 (1 H, d, J = 8.2 Hz) 7.97 |
| | (1H, d, J = 2.8 Hz) 7.88 (1H, d, J = 8.9 Hz) 7.81-7.76 (2H, m) 7.76-7.71 (2H, m) |
| | 7.44-7.40 (2H, m) 7.27-7.22 (2H, m) 7.11 (1H, dd, J = 8.9; 2.8 Hz) 3.58 (2H, d, |
| | J = 6.5 Hz) 0.99-0.89 (1H, m) 0.34-0.27 (2H, m) 0.04-(-0.01) (2H, m) |
| 1:15 | 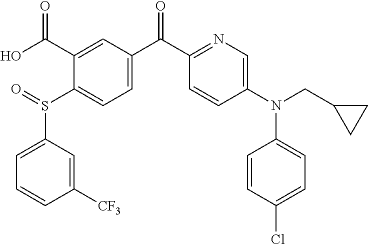 |
| | 5-{5-[(4-Chlorophenyl)cyclopropylmethylamino]pyridine-2-carbonyl}-2-(3-tri- |
| | fluoromethylphenylsulfinyl)benzoic acid |
| | 8.39 (1H, d, J = 1.7 Hz) 8.33 (1 H, dd, J = 8.2; 1.7 Hz) 8.28 (1 H, d, J = 8.2 Hz) 7.99- |
| | 7.96 (2H, m) 7.88 (1 H, d, J = 8.9 Hz) 7.85-7.81 (1H, m) 7.76-7.71 (1H, m) 7.63- |
| | 7.57 (1H, m) 7.44-7.39 (2H, m) 7.27-7.22 (2H, m) 7.11 (1 H, dd, J = 8.9; 2.8 Hz) |
| | 3.58 (2H, d, J = 6.5 Hz) 0.99-0.89 (1H, m) 0.34-0.27 (2H, m) 0.04-(-0.01) (2H, m) |
| 1:16 | 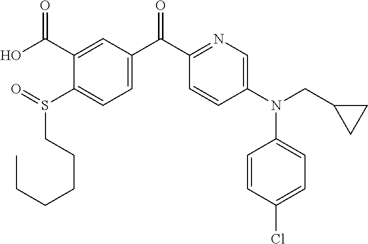 |
| | 5-{5-[(4-Chlorophenyl)cyclopropylmethylamino]pyridine-2-carbonyl}-2-(1-hexyl- |
| | sulfinyl)benzoic acid |
| | 8.58 (1H, d, J = 1.6 Hz) 8.40 (1H, dd, J = 8.2; 1.6 Hz) 8.15 (1 H, d, J = 8.2 Hz) |
| | 8.13 (1H, d, J = 2.9 Hz) 8.02 (1H, d, J = 8.9 Hz) 7.58-7.52 (2H, m) 7.42-7.36 (2H, |
| | m) 7.25 (1H, dd, J = 8.9; 2.9 Hz) 3.72 (2H, d, J = 6.5 Hz) 3.21-3.13 (1H, m) 2.69- |
| | 2.58 (1H, m) 1.86-1.73 (1H, m) 1.59-1.48 (1H, m) 1.46-1.33 (2H, m) 1.30-1.19 |
| | (4H, m) 1.13-1.04 (1H, m) 0.90-0.78 (3H, m) 0.49-0.40 (2H, m) 0.19-0.12 (2H, m) |
| 1:17 | 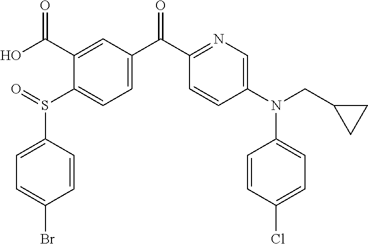 |
| | 2-(4-Bromophenylsulfinyl)-5-{5-[(4-chlorophenyl)cyclopropylmethylamino]- |
| | pyridine-2-carbonyl}benzoic acid |
| | 8.51 (1H, d, J = 1.1 Hz) 8.44 (1H, dd, J = 8.1; 1.1 Hz) 8.37 (1H, d, J = 8.1 Hz) 8.11 |
| | (1H, d, J = 2.6 Hz) 8.01 (1 H, d, J = 8.9 Hz) 7.73-7.66 (2H, m) 7.65-7.59 (2H, m) |
| | 7.58-7.52 (2H, m) 7.41-7.35 (2H, m) 7.24 (1H, dd, J = 8.9; 2.6 Hz) 3.71 (2H, d, |
| | J = 6.5 Hz) 1.12-1.03 (1H, m) 0.48-0.39 (2H, m) 0.19-0.10 (2H, m) |
| 1:18 | 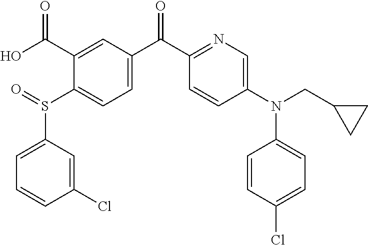 |
| | 2-(3-Chlorophenylsulfinyl)-5-{5-[(4-chlorophenyl)cyclopropylmethylamino]- |
| | pyridine-2-carbonyl}benzoic acid |
| | 8.51 (1H, d, J = 1.5 Hz) 8.45 (1 H, dd, J = 8.2; 1.5 Hz) 8.38 (1 H, d, J = 8.2 Hz) 8.11 |
| | (1H, d, J = 2.9 Hz) 8.01 (1 H, d, J = 8.9 Hz) 7.75-7.72 (1 H, m) 7.66-7.62 (1 H, m) |
| | 7.58-7.49 (4H, m) 7.40-7.35 (2H, m) 7.24 (1H, dd, J = 8.9; 2.9 Hz) 3.71 (2H, d, |
| | J = 6.5 Hz) 1.13-1.04 (1H, m) 0.47-0.40 (2H, m) 0.17-0.12 (2H, m) |
| 1:19 | 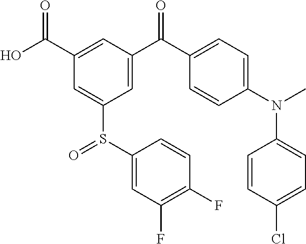 |
| | 3-{4-[(4-Chlorophenyl)(methyl)amino]benzoyl}-5-(3,4-difluorophenylsulfinyl)- |
| | benzoic acid |
| | 13.70 (1H, s) 8.46-8.45 (1H, m) 8.23-8.23 (1H, m) 8.20-8.19 (1H, m) 8.02-7.99 |
| | (1 H, m) 7.75-7.74 (1 H, m) 7.69-7.64 (1 H, m) 7.60-7.59 (2H, m) 7.52-7.50 (2H, |
| | m) 7.34-7.33 (2H, m) 6.85-6.83 (2H, m) 3.36 (3H, s) |
| 1:20 | 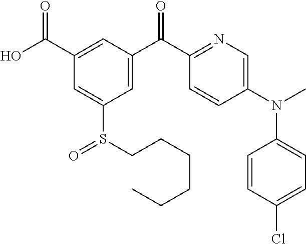 |
| | 3-{5-[(4-Chlorophenyl)methylamino]pyridine-2-carbonyl}-5-(1-hexylsulfinyl)- |
| | benzoic acid |
| | 8.62-8.58 (1H, m) 8.39-8.34 (2H, m) 8.19 (1H, d, J = 3.0 Hz) 8.05 (1H, d, J = 9.0 |
| | Hz) 7.59-7.52 (2H, m) 7.45-7.38 (2H, m) 7.33 (1H, dd, J = 9.0; 3.0 Hz) 3.43 (3H, |
| | s) 3.10-3.02 (1H, m) 2.87-2.80 (1H, m) 1.71-1.62 (1H, m) 1.49-1.40 (1H, m) |
| | 1.39-1.30 (2H, m) 1.27-1.18 (4H, m) 0.86-0.79 (3H, m) |
| 1:21 | 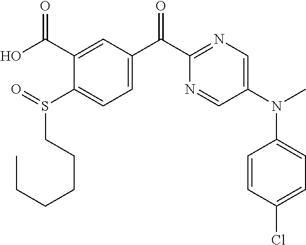 |
| | 5-(5-((4-Chlorophenyl)(methyl)amino)pyrimidine-2-carbonyl)-2-(1-hexylsulfinyl)- |
| | benzoic acid |
| | 8.51-8.43 (3H, m) 8.19-8.12 (1H, m) 8.06 (1H, d, J = 8.0 Hz) 7.59-7.52 (2H, m) |
| | 7.49-7.43 (2H, m) 3.45 (3H, s) 3.32-3.26 (1 H, m, overlapped with water) 2.67- |
| | 2.59 (1H, m, overlapped with DMSO) 1.83-1.71 (1H, m) 1.55-1.44 (1H, m) 1.43- |
| | 1.18 (6H, m) 0.89-0.80 (3H, m) |
| 1:22 | 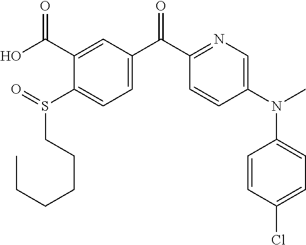 |
| | 5-{5-[(4-Chlorophenyl)methylamino]pyridine-2-carbonyl}-2-(1-hexylsulfinyl)- |
| | benzoic acid |
| | 8.54-8.50 (1H, m) 8.32-8.26 (1H, m) 8.21 (1H, d, J = 2.9 Hz) 8.10 (1H, d, J = 8.0 |
| | Hz) 8.00 (1H, d, J = 8.9 Hz) 7.56-7.50 (2H, m) 7.42-7.36 (2H, m) 7.30 (1 H, dd, |
| | J = 8.9; 2.9 Hz) 3.41 (3H, s) 3.25-3.18 (1H, m) 2.65-2.58 (1H, m) 1.82-1.72 (1H, |
| | m) 1.55-1.47 (1 H, m) 1.39-1.21 (6H, m) 0.86-0.81 (3H, m) |

### Example 2:1

5-{4-[(4-Chlorophenyl)(methyl)amino]benzoyl}-2-(1-hexylsulfanyl)benzoic acid

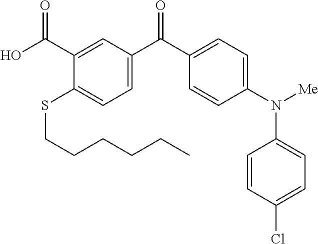

The title compound was prepared from 5-{4-[(4-chlorophenyl)(methyl)amino]-benzoyl}-2-(1-hexylsulfanyl)benzoic acid methyl ester (see Example 1:1, step (c)) in accordance with Example 1:1, step (e). ¹H NMR (DMSO-d₆) δ: 8.12 (1H, d, J=2.0 Hz) 7.76 (1H, dd, J=8.8; 2.0 Hz) 7.62-7.60 (2H, m) 7.51-7.45 (3H, m) 7.30-7.27 (2H, m) 6.86-6.84 (2H, m) 3.32 (3H, s, overlapped with water) 2.95 (2H, t, J=7.6 Hz) 1.66-1.58 (2H, m) 1.45-1.37 (2H, m) 1.27-1.25 (4H, m) 0.86-0.82 (3H, m)

### Examples 2:2-2:4

The title compounds were prepared from 5-{4-[(4-chlorophenyl)(methyl)amino]-benzoyl}-2-fluorobenzoic acid methyl ester (see Example 1:1, step (b)) and the appropriate thiol, followed by hydrolysis in accordance with Example 1:1, steps (c) and (e), see Table 2.

### Examples 2:5-2:6

The title compounds were prepared from 5-{5-[(4-chlorophenyl)methylamino]-pyridine-2-carbonyl}-2-fluorobenzoic acid methyl ester (see Example 1:13, step (d)) and the appropriate thiol followed by hydrolysis in accordance with Example 1:1, steps (c) and (e), see Table 2.

### Examples 2:7-2:8

The title compounds were prepared from 5-{4-[(4-chlorophenyl)cyclopropyl-methylamino]benzoyl}-2-fluorobenzoic acid methyl ester (see Example 1:11, step (c)) and the appropriate thiol followed by hydrolysis in accordance with Example 1:1, steps (c) and (e), see Table 2.

### Examples 2:9-2:20

The title compounds were prepared from 5-{5-[(4-chlorophenyl)cyclopropyl-methylamino]pyridine-2-carbonyl}-2-fluorobenzoic acid methyl ester (see Example 1:14, step (a)) and the appropriate thiol, followed by hydrolysis in accordance with Example 1:1, steps (c) and (e), see Table 2.

### Example 2:21

### 5-[5-(3-Chlorobenzyloxy)pyridine-2-carbonyl]-2-(3,4-difluorophenylsulfanyl)-benzoic acid

### (a) 2-Fluoro-5-(5-methoxypicolinoyl)benzoic acid methyl ester

The sub-title compound was prepared from 2-fluoro-5-iodobenzoic acid methyl ester and 2-cyano-5-methoxypyridine 5-bromo-2-cyanopyrimidine in accordance with Example 1:1, step (a).

### (b) 2-Fluoro-5-(5-hydroxypicolinoyl)benzoic acid methyl ester

AlCl₃ (2.28 g, 16.6 mmol) was added to 2-fluoro-5-(5-methoxypicolinoyl)benzoic acid methyl ester (0.8 g, 2.76 mmol) in CH₂Cl₂ (70 mL). The mixture was stirred for 2.5 h at rt, for 2 days at 40° C. and for 3 days at rt. Extractive workup (CH₂Cl₂, water, brine), drying (Na₂SO₄), concentration and re-crystallization from EtOAc and petroleum ether gave the sub-title compound. Yield: 0.7 g (92%).

### (c) 5-(5-(3-Chlorobenzyloxy)picolinoyl)-2-fluorobenzoic acid methyl ester

NaH (60% in mineral oil, 116 mg, 2.8 mmol) was added to 2-fluoro-5-(5-hydroxy-picolinoyl)benzoic acid methyl ester (0.70 g, 2.55 mmol) in DMF at 0° C. The mixture was stirred at 0° C. for 20 min and 1-chloro-3-(chloromethyl)benzene (0.355 g, 2.8 mmol) was added. After 20 h at rt, NaI (57 mg, 0.38 mmol) and an additional portion of 1-chloro-3-(chloromethyl)benzene (0.355 g, 2.8 mmol) was added. The mixture was stirred at rt for 1 day. Extractive workup (EtOAc, water, brine), drying (Na₂SO₄), concentration and purification by chromatography gave the sub-title compound. Yield: 0.65 g (64%).

### (d) 5-[5-(3-Chlorobenzyloxy)pyridine-2-carbonyl]-2-(3,4-difluorophenyl-sulfanyl)benzoic acid

The title compound was prepared from 5-(5-(3-chlorobenzyloxy)picolinoyl)-2-fluorobenzoic acid methyl ester and 3,4-difluorothiophenol, followed by hydrolysis, in accordance with Example 1:1, steps (c) and (e), see Table 2.

### Example 2:22

### 5-[5-(3-Chlorobenzyloxy)pyridine-2-carbonyl]-2-(4-methoxyphenylsulfanyl)benzoic acid

The title compound was prepared from 5-(5-(3-chlorobenzyloxy)picolinoyl)-2-fluorobenzoic acid methyl ester and 4-methoxythiophenol in accordance with Example 2: 21, see Table 2.

### Examples 2:23-2:24

The title compounds were prepared from 3-{5-[(4-chlorophenyl)methylamino]pyridine-2-carbonyl}-5-iodobenzoic acid methyl ester and the appropriate thiol in accordance with Example 1:20, step (c) and hydrolysis in accordance with Example 1:19 step (f), see Table 2.

### Examples 2:25-2:30

The title compounds were prepared in accordance with Example 1:19, steps (d) and (f) from 3-bromo-5-[4-(4-chlorophenyl(methyl)amino)benzoyl]benzoic acid methyl ester and the appropriate thiol, see Table 2.

TABLE 2

| | Chemical structure |
|---|---|
| | Name |
| Ex. | ¹H-NMR (DMSO-d₆, δ) |
| 2:2 | 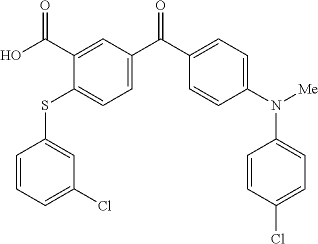 |
| | 5-{4-[(4-Chlorophenyl)(methyl)amino]benzoyl}-2- |
| | (3-chlorophenylsulfanyl)-benzoic acid |
| | 8.17 (1H, d, J = 2.1 Hz) 7.68-7.67 (1H, m) 7.65 (1H, d, |
| | J = 2.1 Hz) 7.60-7.54 (5H, m) 7.46-7.44 (2H, m) 7.29-7.26 |
| | (2H, m) 6.84-6.82 (3H, m) 3.31 (3H, s) |
| 2:3 | 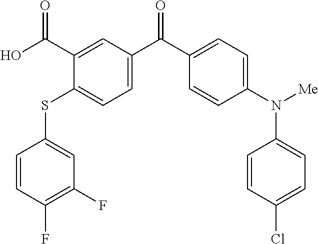 |
| | 5-{4-[(4-Chlorophenyl)(methyl)amino]benzoyl}-2 |
| | -(3,4-difluorophenyl-sulfanyl)benzoic acid |
| | 13.8-13.3 (1 H, br s) 8.24 (1H, d, J = 1.8 Hz) 7.83 |
| | (1 H, ddd, J = 9.9; 7.8; 1.8 Hz) 7.76-7.58 (4H, m) 7.57-7.46 |
| | (3H, m) 7.39-7.28 (2H, m) 6.94-6.82 (3H, m) 3.4 (3H, s) |
| 2:4 | 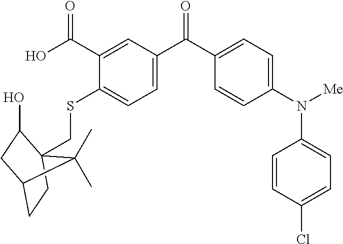 |
| | 5-{4-[(4-Chlorophenyl)methylamino]benzoyl}-2-(2-hydroxy-7,7- |
| | dimethyl-bicyclo[2.2.1]-1-heptylmethylsulfanyl)benzoic acid |
| | 8.10 (1H, d, J = 1.9 Hz) 7.74 (1H, dd, J = 8.5; 1.9 Hz) |
| | 7.67-7.62 (2H, m) 7.59 (1H, d, J = 8.5 Hz) 7.51-7.46 |
| | (2H, m) 7.35-7.29 (2H, m) 6.92-6.86 (2H, m) 5.4-4.8 |
| | (1H, br s) 3.78-3.72 (1H, m) 3.35 (3H, s) 3.17 (1H, d, |
| | J = 10.3 Hz) 2.80 (1H, d, J = 10.3 Hz) 1.70-1.55 (5H, m) |
| | 1.22-1.15 (1H, m) 1.09 (3H, s) 1.05-0.98 (1 H, m) 0.90 (3H, s) |
| 2:5 | 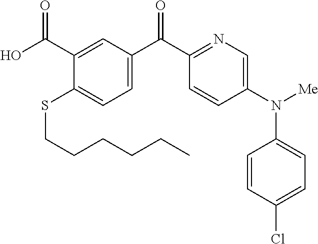 |
| | 5-{5-[(4-Chlorophenyl)methylamino]pyridine-2-carbonyl}2- |
| | (1-hexyl-sulfanyl)benzoic acid |
| | 8.51-8.44 (1H, m) 8.20 (1H, d, J = 2.7 Hz) 8.03-8.97 (1H, m) 7.92 |
| | (1H, d, J = 8.8 Hz) 7.55-7.49 (2H, m) 7.41-7.33 (3H, m) 7.31 |
| | (1 H, dd, J = 9.0; 2.8 Hz) 3.40 (3H, s) 2.92-2.85 (2H, m) 1.67-1.59 |
| | (2H, m) 1.48-1.41 (2H, m) 1.33-1.27 (4H, m) 0.90-0.85 (3H, m) |
| 2:6 | 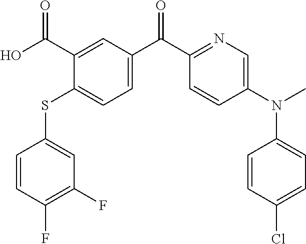 |
| | 5-{5-[(4-Chlorophenyl)methylamino]pyridine-2-carbonyl}-2- |
| | (3,4-difluoro-phenylsulfanyl)benzoic acid |
| | 8.53-8.50 (1H, m) 8.18 (1H, d, J = 2.7 Hz) 7.91 (1H, d, J = |
| | 8.8 Hz) 7.88-7.82 (1H, m) 7.74-7.66 (1H, m) 7.63-7.55 (1H, m) |
| | 7.54-7.48 (2H, m) 7.46-7.40 (1H, m) 7.39-7.34 (2H, m) 7.28 |
| | (1 H, dd, J = 8.8; 2.7 Hz) 6.7 (1H, d, J = 8.4 Hz) 3.38 (3H, s) |
| 2:7 | 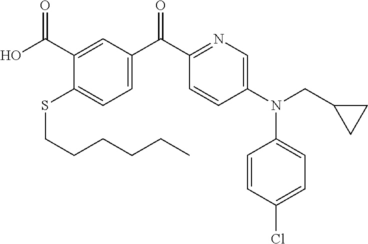 |
| | 5-{4-[(4-Chlorophenyl)cyclopropylmethylamino]benzoyl}- |
| | 2-(1-hexyl-sulfanyl)benzoic acid |
| | 8.15 (1H, d, J = 1.9 Hz) 7.78 (1H, dd, J = 8.2; 1.9 Hz) |
| | 7.68-7.57 (2H, m) 7.57-7.44 (3H, m) 7.39-7.24 (2H, m) |
| | 6.89-6.75 (2H, m) 3.65 (2H, d, J = 6.6 Hz) 2.97 (2H, t, J = 7.2 Hz) |
| | 1.72-1.57 (2H, m) 1.52-1.37 (2H, m) 1.37-1.21 (4H, m) 1.14-1.00 |
| | (1H, m) 0.95-0.79 (3H, m) 0.51-0.35 (2H, m) 0.19-0.06 (2H, m) |
| 2:8 | 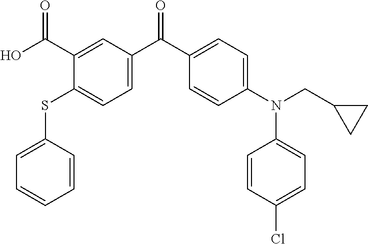 |
| | 5-{4-[(4-Chlorophenyl)cyclopropylmethylamino]benzoyl}- |
| | 2-phenylsulfanyl-benzoic acid |
| | 8.19 (1H, d, J = 1.9 Hz) 7.68-7.52 (8H, m) 7.52-7.45 (2H, m) |
| | 7.35-7.25 (2H, m) 6.86-6.72 (3H, m) 3.64 (2H, d, J = 6.6 Hz) |
| | 1.13-1.01 (1H, m) 0.48-0.35 (2H, m) 0.18-0.05 (2H, m) |
| 2:9 | 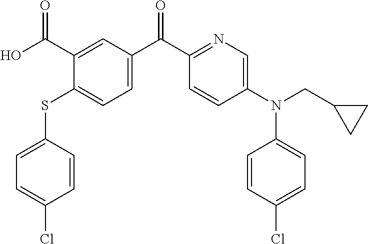 |
| | 5-{5-[(4-Chlorophenyl)cyclopropylmethylamino]pyridine-2- |
| | carbonyl}-2-(4-chlorophenylsulfanyl)benzoic acid |
| | 13.5-13.3 (1H, br s) 8.48 (1H, d, J = 1.9 Hz) 7.97 |
| | (1H, d, J = 2.8 Hz) 7.88 (1H, dd, J = 8.6; 1.9 Hz) |
| | 7.81 (1H, d, J = 8.9 Hz) 7.52-7.46 (4H, m) 7.44- |
| | 7.39 (2H, m) 7.27-7.21 (2H, m) 7.11 (1H, |
| | dd, J = 8.9; 2.8 Hz) 6.67 (1H, d, J = 8.6 Hz) 3.57 (2H, d, |
| | J = 6.5 Hz) 1.00-0.91 (1H, m) 0.34-0.28 (2H, m) |
| | 0.04-(-0.01) (2H, m) |
| 2:10 | 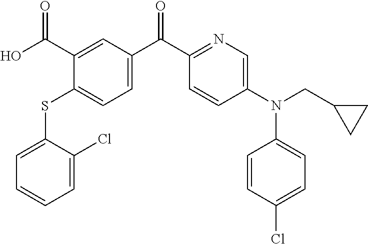 |
| | 5-{5-[(4-Chlorophenyl)cyclopropylmethylamino]pyridine-2- |
| | carbonyl}-2-(2-chlorophenylsulfanyl)benzoic acid |
| | 13.5-13.3 (1 H, br s) 8.51 (1H, d, J = 1.9 Hz) 7.98 |
| | (1 H, d, J = 2.8 Hz) 7.89 (1H, dd, J = 8.6; 1.9 Hz) 7.81 |
| | (1H, d, J = 8.9 Hz) 7.68-7.64 (1H, m) 7.63-7.59 (1H, m) |
| | 7.50-7.44 (1H, m) 7.43-7.36 (3H, m) 7.26-7.21 (2H, m) 7.10 |
| | (1H, dd, J = 8.9; 2.8 Hz) 6.51 (1H, d, J = 8.6 Hz) 3.57 (2H, d, |
| | J = 6.5 Hz) 0.99-0.89 (1H, m) 0.34-0.27 (2H, m) |
| | 0.04-(-0.01) (2H, m) |
| 2:11 | 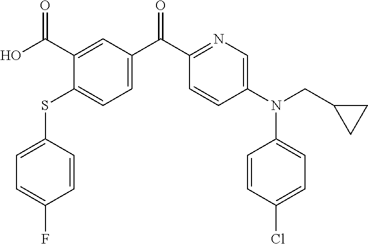 |
| | 5-{5-[(4-Chlorophenyl)cyclopropylmethylamino]pyridine-2- |
| | carbonyl}-2-(4-fluorophenylsulfanyl)benzoic acid |
| | 8.49 (1H, d, J = 1.9 Hz) 7.97 (1H, d, J = 2.8 Hz) 7.88 (1H, dd, |
| | J = 8.6 1.9 Hz) 7.81 (1H, d, J = 8.9 Hz) |
| | 7.57-7.51 (2H, m) 7.43-7.39 (2H, m) 7.31-7.21 |
| | (4H, m) 7.10 (1H, dd, J = 8.9 2.8 Hz) 6.61 |
| | (1H, d, J = 8.6 Hz) 3.57 (2H, d, J = 6.5 Hz) 0.99-0.89 (1H, m) |
| | 0.34-0.28 (2H, m) 0.04-(-0.01) (2H, m) |
| 2:12 | 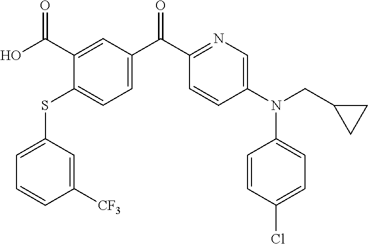 |
| | 5-{5-[(4-Chlorophenyl)cyclopropylmethylamino]pyridine-2- |
| | carbonyl}-2-(3-trifluoromethylphenylsulfanyl)benzoic acid |
| | 13.5-13.3 (1 H, br s) 8.48 (1H, d, J = 1.9 Hz) 7.96 (1 H, d, |
| | J = 2.8 Hz) 7.89 (1H, dd, J = 8.6; 1.9 Hz) 7.83-7.74 (4H, m) |
| | 7.68-7.61 (1 H, m) 7.43-7.37 (2H, m) 7.26-7.19 (2H, m) |
| | 7.09 (1H, dd, J = 8.9; 2.8 Hz) 6.65 (1H, d, J = 8.6 Hz) |
| | 3.56 (2H, d, J = 6.5 Hz) 0.99-0.89 (1 H, m) 0.34-0.25 |
| | (2H, m) 0.04-(-0.03) (2H, m) |
| 2:13 | 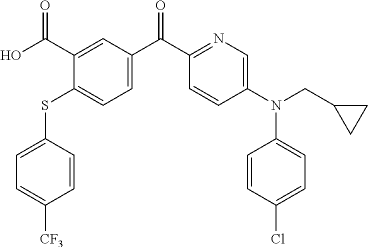 |
| | 5-{5-[(4-Chlorophenyl)cyclopropylmethylamino]pyridine-2- |
| | carbonyl}-2-(4-trifluoromethylphenylsulfanyl)benzoic acid |
| | 8.48-8.44 (1H, m) 7.98-7.94 (1H, m) 7.91-7.85 (1H, m) |
| | 7.80 (1H, d, J = 8.9 Hz) 7.75-7.70 (2H, m) 7.68-7.62 (2H, m) |
| | 7.43-7.37 (2H, m) 7.25-7.20 (2H, m) 7.09 (1H, dd, |
| | J = 8.9; 2.8 Hz) 6.75 (1H, d, J = 8.6 Hz) 3.56 (2H, d, J = 6.5 |
| | Hz) 0.99-0.89 (1H, m) 0.34-0.25 (2H, m) 0.04-(-0.03) (2H, m) |
| 2:14 | 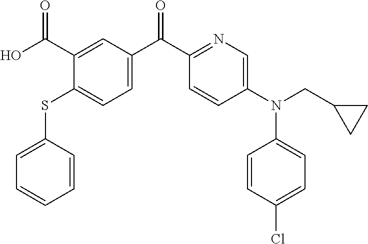 |
| | 5-{5-[(4-Chlorophenyl)cyclopropylmethylamino]pyridine-2-carbonyl}- |
| | 2-phenylsulfanylbenzoic acid |
| | 13.7-13.3 (1H, br s) 8.61 (1H, d, J = 1.8 Hz) 8.10 |
| | (1H, d, J = 2.7 Hz) 7.97 (1H, dd, J = 8.5; 1.8 Hz) |
| | 7.93 (1H, d, J = 8.9 Hz) 7.64-7.50 (7H, m) 7.39- |
| | 7.33 (2H, m) 7.23 (1 H, dd, J = 8.9; 2.7 Hz) 6.75 |
| | (1 H, d, J = 8.5 Hz) 3.70 (2H, d, J = 6.5 Hz) 1.13-1.02 (1H, m) |
| | 0.46-0.39 (2H, m) 0.17-0.11 (2H, m) |
| 2:15 | 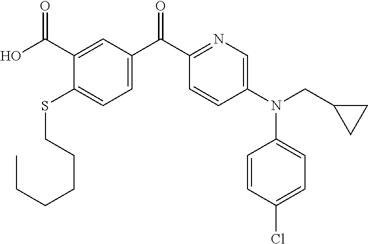 |
| | 5-{5-[(4-Chlorophenyl)cyclopropylmethylamino]pyridine-2- |
| | carbonyl}-2-(1-hexylsulfanyl)benzoic acid |
| | 13.2-13.1 (1H, br s) 8.58 (1H, d, J = 1.9 Hz) 8.16 |
| | (1H, dd, J = 8.5; 1.7 Hz) 8.13 (1H, d, J = 2.9 Hz) 7.95 (1H, d, |
| | J = 9.0 Hz) 7.57-7.52 (2H, m) 7.50 (1H, d, J = 8.5 Hz) |
| | 7.40-7.35 (2H, m) 7.25 (1H, dd, J = 9.0; 2.9 Hz) 3.71 (2H, d, |
| | J = 6.5 Hz) 2.98 (2H, t, J = 7.2 Hz) 1.70-1.60 (2H, m) |
| | 1.50-1.39 (2H, m) 1.34-1.22 (4H, m) 1.14-1.04 (1H, m) |
| | 0.91-0.83 (3H, m) 0.47-0.41 (2H, m) 0.18-0.12 (2H, m) |
| 2:16 | 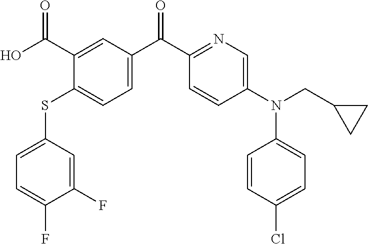 |
| | 5-{5-[(4-Chlorophenyl)cyclopropylmethylamino]pyridine-2- |
| | carbonyl}-2-(3,4-difluorophenylsulfanyl)benzoic acid |
| | 13.6-13.4 (1H, br s) 8.62 (1H, d, J = 1.9 Hz) 8.10 |
| | (1H, d, J = 2.9 Hz) 8.03 (1H, dd, J = 8.5; 1.9 Hz) |
| | 7.94 (1H, d, J = 8.9 Hz) 7.82-7.75 (1H, m) 7.67- |
| | 7.59 (1H, m) 7.56-7.52 (2H, m) 7.51-7.46 (1H, m) 7.39-7.34 |
| | (2H, m) 7.24 (1H, dd, J = 8.9; 2.9 Hz) 6.82 (1H, d, |
| | J = 8.5 Hz) 3.70 (2H, d, J = 6.5 Hz) |
| | 1.12-1.03 (1H, m) 0.47-0.40 (2H, m) 0.18-0.10 (2H, m) |
| 2:17 | 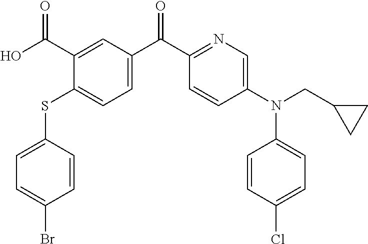 |
| | 2-(4-Bromophenylsulfanyl)-5-{5-[(4-chlorophenyl) |
| | cyclopropylmethyl-amino]pyridine-2-carbonyl}benzoic acid |
| | 13.5-13.4 (1H, br s) 8.61 (1H, d, J = 1.7 Hz) 8.10 (1H, d, |
| | J = 2.7 Hz) 8.02 (1H, dd, J = 8.6 1.7 Hz) 7.94 (1 H, d, J = 9.0 Hz) |
| | 7.77-7.71 (2H, m) 7.58-7.51 (4H, m) 7.39-7.34 (2H, m) |
| | 7.23 (1 H, dd, J = 9.0 2.7 Hz) 6.81 (1 H, |
| | d, J = 8.6 Hz) 3.70 (2H, d, J = 6.5 Hz) |
| | 1.13-1.03 (1H, m) 0.47-0.39 (2H, m) 0.17-0.11 (2H, m) |
| 2:18 | 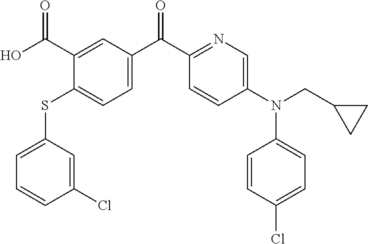 |
| | 5-{5-[(4-Chlorophenyl)cyclopropylmethylamino]pyridine-2- |
| | carbonyl}-2-(3-chlorophenylsulfanyl)benzoic acid |
| | 13.6-13.4 (1H, br s) 8.61 (1H, d, J = 1.9 Hz) 8.11 |
| | (1H, d, J = 2.8 Hz) 8.03 (1H, dd, J = 8.5; 1.9 Hz) 7.94 |
| | (1H, d, J = 9.0 Hz) 7.70-7.67 (1H, m) 7.65-7.60 (1H, m) 7.59-7.51 |
| | (4H, m) 7.39-7.33 (2H, m) 7.23 (1H, dd, J = 9.0 2.8 Hz) 6.82 |
| | (1H, d, J = 8.5 Hz) 3.70 (2H, d, J = 6.5 Hz) 1.13-1.02 (1H, m) |
| | 0.47-0.40 (2H, m) 0.17-0.11 (2H, m) |
| 2:19 | 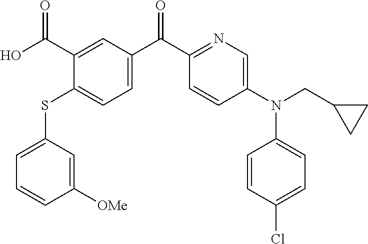 |
| | 5-{5-[(4-Chlorophenyl)cyclopropylmethylamino]pyridine-2- |
| | carbonyl}-2-(3-methoxy-phenylsulfanyl)-benzoic acid |
| | 13.6-13.2 (1H, br s) 8.60 (1H, d, J = 2.0 Hz) 8.10 (1H, d, J = |
| | 2.9 Hz) 7.99 (1H, dd, J = 8.5; 2.0 Hz) 7.93 (1H, d, |
| | J = 9.0 Hz) 7.56-7.50 (2H, m) 7.49-7.43 (1H, m) 7.39-7.33 |
| | (2H, m) 7.23 (1H, dd, J = 9.0; 2.9 Hz) 7.18-7.14 (2H, m) |
| | 7.13-7.09(1H, m) 6.81 (1H, d, J = 8.5 Hz) 3.78 (3H, s) |
| | 3.69 (2H, d, J = 6.5 Hz) 1.13-1.02 (1H, m) 0.47-0.39 |
| | (2H, m) 0.18-0.11 (2H, m) |
| 2:20 | 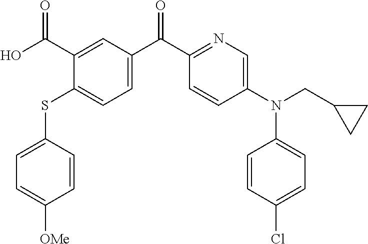 |
| | 5-{5-[(4-Chlorophenyl)cyclopropylmethylamino]pyridine-2- |
| | carbonyl}-2-(4-methoxyphenylsulfanyl)benzoic acid |
| | 13.4-13.3 (1H br s) 8.61 (1H, d, J = 2.0 Hz) 8.10 |
| | (1H, d, J = 2.9 Hz) 7.99 (1H, dd, J = 8.6; 2.0 Hz) |
| | 7.93 (1H, d, J = 9.0 Hz) 7.56-7.50(4H, m) 7.39-7.35 |
| | (2H, m) 7.24 (1H, dd, J = 9.0 2.9 Hz) 7.14-7.09 |
| | (2H, m) 6.73 (1H, d, J = 8.6 Hz) 3.84 (3H, s) 3.70 (2H, d, |
| | J = 6.5 Hz) 1.14-1.04 (1H, m) 0.47-0.40 (2H, m) |
| | 0.18-0.11 (2H, m) |
| 2:21 | 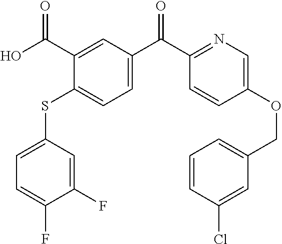 |
| | 5-[5-(3-Chlorobenzyloxy)pyridine-2-carbonyl]-2-(3,4- |
| | difluorophenyl-sulfanyl)benzoic acid |
| | 13.56 (1H, s) 8.61 (1H, d, J = 1.9 Hz) 8.48 (1H, d, J = 2.9 Hz) |
| | 8.08 (1H, d, J = 8.8 Hz) 8.02 (1H, dd, J = 8.6; 2.0 Hz) |
| | 7.85-7.76 (1H, m) 7.70 (1H, dd, J = 8.8; 2.9 Hz) |
| | 7.68-7.56 (2H, m) 7.53-7.39 (4H, m) 6.86 (1H, d, J = 8.6 Hz) |
| | 5.32 (2H, s) |
| 2:22 | 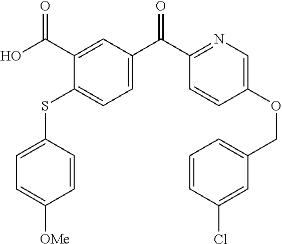 |
| | 5-[5-(3-Chlorobenzyloxy)pyridine-2-carbonyl]-2- |
| | (4-methoxyphenyl-sulfanyl)benzoic acid |
| | 13.6-13.3 (1 H, br s) 8.58 (1H, d, J = 2.0 Hz) 8.47 |
| | (1 H, d, J = 2.8 Hz) 8.07(1H, d, J = 8.9 Hz) 7.96 |
| | (1 H, dd, J = 8.6; 2.0 Hz) 7.69 (1 H, dd, J = 8.9; 2.8 Hz) 7.61- |
| | 7.56 (1H, m) 7.55-7.50 (2H, m) 7.48-7.41 (3H, m) 7.15-7.08 |
| | (2H, m) 6.76 (1H, d, J = 8.6 Hz) 5.32 (2H, s), 3.84 (3H, s) |
| 2:23 | 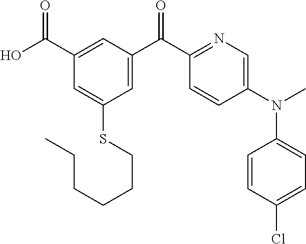 |
| | 3-{5-[(4-Chlorophenyl)methylamino]pyridine-2-carbonyl}-5- |
| | (1-hexyl)-sulfanylbenzoic acid |
| | 8.29-8.25 (1H, m) 8.20 (1H, d, J = 3.0 Hz) 8.10-8.07 |
| | (1H, m) 8.02 (1H, d, J = 9.0 Hz) 8.00-7.97 (1H, m) 7.58-7.52 |
| | (2H, m) 7.44-7.38 (2H, m) 7.32 (1H, dd, J = 9.0; 3.0 Hz) |
| | 3.43 (3H, s) 3.07-3.02 (2H, m) 1.67-1.59 (2H, m) |
| | 1.45-1.36 (2H, m) 1.27-1.23 (4H, m) 0.88-0.83 (3H, m) |
| 2:24 | 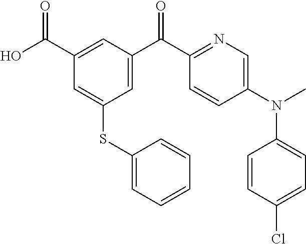 |
| | 3-{5-[(4-Chlorophenyl)methylamino]pyridine-2-carbonyl}-5- |
| | phenylsulfanyl-benzoic acid |
| | 8.39-8.36 (1H, m) 8.12 (1H, d, J = 3.0 Hz) 8.10-8.08 |
| | (1H, m) 7.99 (1H, d, J = 9.0 Hz) 7.95-7.92 (1H, m) 7.60-7.55 |
| | (2H, m) 7.53-7.49 (2H, m) 7.48-7.43 (2H, m) 7.42-7.38 |
| | (3H, m) 7.29 (1H, dd, J = 9.0; 3.0 Hz) 3.42 (3H, s) |
| 2:25 | 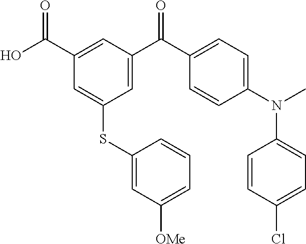 |
| | 3-(4-((4-Chlorophenyl)(methyl)amino)benzoyl)-5-(3- |
| | methoxyphenylsulfanyl)benzoic acid |
| | 13.42 (1H, s) 7.98-7.98 (1H, m) 7.95-7.94 (1H, m) 7.65-7.74 |
| | (1H, m) 7.60-7.59 (2H, m) 7.52-7.50 (2H, m) 7.38-7.31 (3H, m) |
| | 7.05-7.03 (2H, m) 6.98-6.96 (1 H, m) 6.83-6.81 |
| | (2H, m) 3.73 (3H, s) 3.34 (3H, s) |
| 2:26 | 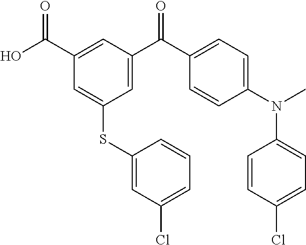 |
| | 3-(4-((4-Chlorophenyl)(methyl)amino)benzoyl)-5-(3- |
| | chlorophenylsulfanyl)benzoic acid |
| | 13.47 (1H, s) 8.04-8.03 (1H, m) 8.00-8.00 (1H, m) 7.70-7.69 |
| | (1H, m) 7.61-7.59 (2H, m) 7.55-7.54 (1H, m) 7.52-7-50 |
| | (2H, m) 7.46-7.41 (3H, m) |
| | 7.33-7.31 (2H, m) 6.84-6-82 (2H, m) 3.35 (3H,s) |
| 2:27 | 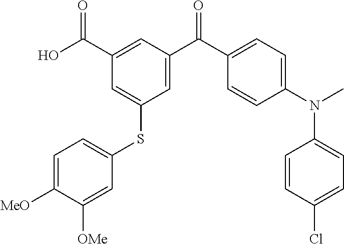 |
| | 3-(4-((4-Chlorophenyl)(methyl)amino)benzoyl)-5-(3,4- |
| | dimethoxyphenylsulfanyl)benzoic acid |
| | 13.36 (1H, m) 7.79-7.88 (1H, m) 7.83-7.82 (1H, m) 7.57-7.56 |
| | (2H, m) 7.54-7.48 (3H, m) 7.32-7-30 (2H, m) 7.16-7.14 (2H, m) |
| | 7.06-7.05 (1H, m) 6.82-6.80 (2H, m) 3.78 (3H, s) |
| | 3.73 (3H, s) 3.34 (3H, s) |
| 2:28 | 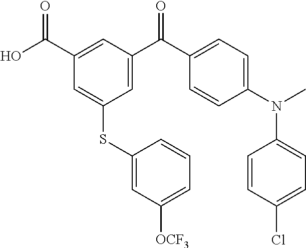 |
| | 3-(4-((4-Chlorophenyl)(methyl)amino)benzoyl)-5-(3- |
| | (trifluoromethoxy)phenylsulfanyl)benzoic acid |
| | 13.48 (1H, s) 8.05-8.04 (1H, m) 8.03-8.02 (1H, m) 7.73-7.73 |
| | (1H, m) 7.62-7.59 (2H, m) 7.58-7.54 (1H, m) 7.51-7.49 |
| | (2H, m) 7.45-7.44 (2H, m) 7.38-7.36 (1H, m) 7.33-7.30 |
| | (2H, m) 6.84-6.81 (2H, m) 3.34 (3H, m) |
| 2:29 | 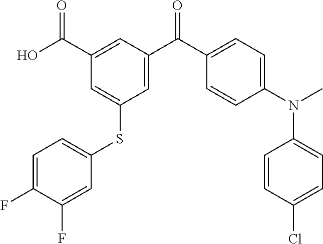 |
| | 3-(4-((4-Chlorophenyl)(methyl)amino)benzoyl)-5-(3,4- |
| | difluorophenylsulfanyl)benzoic acid |
| | 13.47 (1H, s) 8.01-8.00 (1H, m) 7.97-7.96 (1H, m) 7.71-7.67 |
| | (1H, m) 7.66-7.65 (1H, m) 7.61-7.58 (2H, m) 7.56-7.48 (3H, m) |
| | 7.38-7.35 (1H, m) 7.33-7.30 (2H, m) 6.84-6.81 |
| | (2H, m) 3.34 (3H, s) |
| 2:30 | 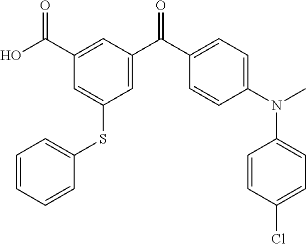 |
| | 3-(4-((4-Chlorophenyl)(methyl)amino)benzoyl)-5- |
| | (phenysulfanyl)benzoic acid |
| | 13.32 (1H, s) 8.43-8.40 (1H, m) 7.70-7.64 (3H, m) 7.59-7.57 |
| | (1H, m) 7.51-7.47 (2H, m) 7.39-7.23 (5H, m) 7.18-7.13 |
| | (1H, m) 7.09-7.03 (1H, m) 6.91-6.85 (2H, m) 3.35 (3H, s) |

### Example 3:1

### 5-{4-[(4-Chlorophenyl)(methyl)amino]benzoyl}-2-(1-hexylsulfonyl)benzoic acid

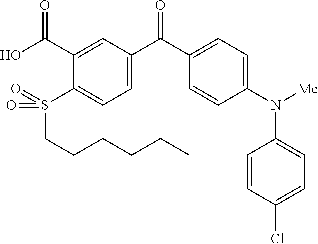

The title compound was prepared from 5-{4-[(4-chlorophenyl)(methyl)amino]-benzoyl}-2-(1-hexylthio)benzoic acid methyl ester (see Example 1:1, step (c)) in accordande with Example 1:1, step (d) using an excess (8 equivalents) of Oxone, followed by hydrolysis in accordance with Example 1:1, step (e).

¹H NMR (DMSO-d₆) δ 8.10 (1H, d, J=8.2 Hz) 7.94 (1H, dd, J=8.2; 1.5 Hz) 7.89 (1H, d, J=1.5 Hz) 7.75-7.61 (2H, m) 7.60-7.46 (2H, m) 7.42-7.28 (2H, m) 6.95-6.82 (2H, m) 3.67-3.58 (2H, m) 3.38 (3H, s) 1.72-1.60 (2H, m) 1.43-1.32 (2H, m) 1.30-1.19 (4H, m) 0.91-0.79 (3H, m).

### Example 3:2

### 5-{4-[(4-Chlorophenyl)(methyl)amino]benzoyl}-2-(4-chlorophenylsulfonyl)benzoic acid

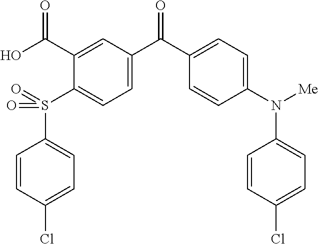

The title compound was synthesized from 5-{4-[(4-chlorophenyl)amino]benzoyl}-2-fluorobenzoic acid methyl ester and 4-chlorothiophenol in accordance with Example 1:1, step (b), followed by oxidation in accordance with Example 3: 1 and hydrolysis in accordance with Example 1:1, step (e). ¹H NMR (DMSO-d₆) δ: 14.1-13.7 (1H, br s) 8.34 (1H, d, J=8.2 Hz) 8.08-7.98 (2H, m) 7.94 (1H, dd, J=8.2; 1.5 Hz) 7.82 (1H, d, J=1.5 Hz) 7.79-7.71 (2H, m) 7.70-7.61 (2H, m) 7.57-7.47 (2H, m) 7.40-7.28 (2H, m) 6.94-6.79 (2H, m) 3.37 (3H, s).

### Example 4:1

### 3-{4-[(4-Chlorophenyl)(methyl)amino]benzoyl}-5-(3,4-difluorophenylsulfinyl)-benzoic acid

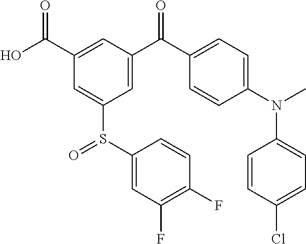

### (a) 3-Bromo-5-{4-[4-chlorophenyl(methyl)amino]benzoyl}benzoic acid methyl ester

i-PrMgCl in THF (22.5 mL, 29 mmol, 1.3 M) was added dropwise to a mixture of 3-bromo-5-iodobenzoic acid methyl ester (8.54 g, 25 mmol) and THF (150 mL) at −15° C. The mixture was stirred at −15° C. for 80 min and cooled to −45° C. 4-[(4-Chlorophenyl)(methyl)amino]benzaldehyde (4.3 g, 17.5 mmol) in THF (30 mL) was added dropwise and the mixture was stirred for 20 min at −45° C. and at rt for 20 h. NH₄Cl (aq, sat) was added. Extractive workup (EtOAc, H₂O, brine) and concentration gave a residue (10 g). A mixture of the residue (8 g, 17.4 mmol), DMF (150 mL) and MnO₂ (32 g, 368 mmol) was stirred at rt for 24 h. Filtration, concentration, crystallization from EtOAc, washing with isohexane and drying gave the sub-title compound. Yield: 6 g (75%).

### (b) 3-{4-[(4-Chlorophenylmethylamino]benzoyl}-5-(3,4-difluoroqhenyl-sulfanyl)benzoic acid methyl ester

A mixture of 3-bromo-5-[4-(4-chlorophenyl(methyl)amino)benzoyl]benzoic acid methyl ester (0.20 g, 0.44 mmol, see step (a) above), i-PrNEt₂ (113 mg, 0.88 mmol), dioxane (2 mL), Pd₂(dba)₃ (10.1 mg, 0.011 mmol), xantphos (12.7 mg, 0.022 mmol) and 3,4-difluorothiophenol (64 mg, 0.44 mmol) was heated at Tx for 20 h. The mixture was filtered through Celite and the solids washed with EtOAc. Concentration of the combined filtrates and purification of the residue by chromatography gave the sub-title compound. Yield: 152 mg (67%).

### (c) 5-{4-[(4-Chlorophenyl)(methyl)amino]benzoyl}-3-(3,4-difluororophenyl-sulfinyl)benzoic acid methyl ester

Oxone (528 mg, 0.86 mmol) in H₂O (7 mL) was added to 3-{4-[(4-chlorophenyl)-methylamino]benzoyl}-5-(3,4-difluorophenylsulfanyl)benzoic acid methyl ester (150 mg 0.29 mmol) in THF (7 mL) at 0° C. The mixture was stirred at 0° C. for 10 min and at rt for 5 h. Extractive workup (EtOAc, H₂O, brine) and purification by chromatography gave the sub-title compound. Yield: 100 mg (65%).

### (d) 5-{4-[(4-Chlorophenyl)(methyl)amino]benzoyl}-3-(3,4-difluororoqhenyl-sulfinyl)benzoic acid

A mixture of 5-{4-[(4-chlorophenyl)(methyl)amino]benzoyl}-3-(3,4-difluororophenyl-sulfinyl)benzoic acid methyl ester (170 mg, 0.33 mmol), NaOH (1 M, aq, 16.4 mL, 1.64 mmol) and EtOH (50 mL) was stirred at 70° C. for 40 min. Acidification with 1 M HCl, extractive workup (EtOAc, H₂O, brine), drying (Na₂SO₄) and concen-tration gave the title product. Yield: 70 mg (42%). 13.70 (1H, s) 8.46-8.45 (1H, m) 8.23-8.23 (1H, m) 8.20-8.19 (1H, m) 8.02-7.99 (1H, m) 7.75-7.74 (1H, m) 7.69-7.64 (1H, m) 7.60-7.59 (2H, m) 7.52-7.50 (2H, m) 7.34-7.33 (2H, m) 6.85-6.83 (2H, m) 3.36 (3H, s). IC₅₀=212 nM.

### Example 4:2

### 3-{5-[(4-Chlorophenyl)(methyl)amino]picolinoyl}-5-(1-hexylsulfinyl)benzoic acid

### (a) 5-[(4-Chlorophenyl)methylamino]pyridine-2-carbaldehyde

The sub-title compound was prepared from 5-bromo-2-formylpyridine and 4-chloro-N-methylaniline. Toluene (e.g. 100 mL) and 4-chloro-N-methylaniline (e.g. 37.8 mmol) may be added to a mixture of Cs₂CO₃ (e.g. 53 mmol), Pd(OAc)₂ (e.g. 1.9 mmol), BINAP (e.g. 2.8 mmol) and 5-bromo-2-formylpyridine (e.g. 37.8 mmol). The mixture may be stirred at 85° C. for 20 h and filtered through Celite. The solids may be washed with EtOAc. The combined filtrates may be concentrated and the residue purified by chromatography to give the sub-title compound.

### (b) 3-{5-[(4-Chlorophenyl)(methyl)amino]picolinoyl-5-iodobenzoic acid methyl ester

The sub-title compound was prepared from 3,5-diiodobenzoic acid methyl ester and 5-[(4-chlorophenyl)methylamino]pyridine-2-carbaldehyde in accordance with Example 4: 1, step (a), followed by oxidation in accordance with Example 4: 1, step (a).

### (c) 3-{5-[(4-Chlorophenyl)(methyl)amino]picolinoyl}-5-(1-hexylsulfanyl)benzoic acid methyl ester

A mixture of 3-{5-[(4-chlorophenyl)(methyl)amino]picolinoyl}-5-iodobenzoic acid methyl ester (0.285 g, 0.56 mmol, see step (b) above), 1-hexanethiol (0.072 g, 0.61 mmol), Pd₂(dba)₃ (0.018 g, 0.02 mmol), bis(2-diphenylphosphinophenyl)ether (0.018 g, 0.034 mmol), potassium tert-butoxide (0.126 g, 1.12 mmol) and toluene (10 mL) was stirred at Tx for 6 h. Filtration through Celite, washing with EtOAc, concentration of the combined filtrates and purification of the residue by chromatography gave the sub-title compound. Yield 0.08 g (28%).

### (d) 3-{5-[(4-Chlorophenyl)(methyl)amino]picolinoyl}-5-(1-hexylsulfinyl)benzoic acid

The title compound was prepared from 3-{5-[(4-chlorophenyl)(methyl)amino]-picolinoyl}-5-(1-hexylsulfanyl)benzoic acid methyl ester by oxidation and hydrolysis in accordance with Example 4: 1, steps (c) and (d), see Table 4.

### Examples 4:3 and 4:4

The title compounds were prepared from 3-{5-[(4-chlorophenyl)(methyl)amino]-picolinoyl}-5-iodobenzoic acid methyl ester and the appropriate thiol in accordance with Example 4: 2, step (c) and hydrolysis in accordance with Example 1:1 (e) (or employed other methods, e.g. EtOH as the solvent, or another suitable base), see Table 4.

### Examples 4:5-4:10

The title compounds were prepared in accordance with Example 4:1, steps (b) and (d) from 3-bromo-5-[4-(4-chlorophenyl(methyl)amino)benzoyl]benzoic acid methyl ester and the appropriate thiol, see Table 4.

TABLE 4

| | Chemical structure | IC₅₀ (nM) |
|---|---|---|
| | Name | |
| Ex. | 1H-NMR (DMSO-d₆, δ) | |
| 4:2 | 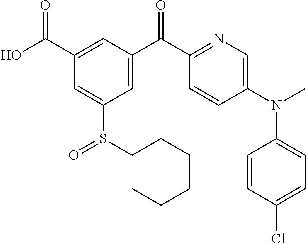 | 404 |
| | 3-{5-[(4-Chlorophenyl)(methyl)amino]picolinoyl}-5-(1-hexylsulfinyl)-benzoic acid | |
| | 8.62-8.58 (1H, m) 8.39-8.34 (2H, m) 8.19 (1H, d, J = 3.0 Hz) 8.05 (1H, d, | |
| | J = 9.0 Hz) 7.59-7.52 (2H, m) 7.45-7.38 (2H, m) 7.33 (1H, dd, J = 9.0; 3.0 | |
| | Hz) 3.43 (3H, s) 3.10-3.02 (1H, m) 2.87-2.80 (1H, m) 1.71-1.62 (1H, m) | |
| | 1.49-1.40 (1H, m) 1.39-1.30 (2H, m) 1.27-1.18 (4H, m) 0.86-0.79 (3H, m) | |
| 4:3 | 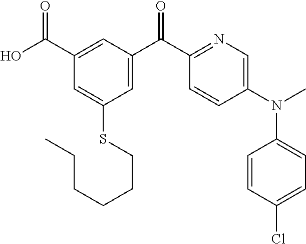 | 215 |
| | 3-{5-[(4-Chlorophenyl)(methyl)amino]picolinoyl}-5-(1-hexyl)-sulfanylbenzoic acid | |
| | 8.29-8.25 (1H, m) 8.20 (1H, d, J = 3.0 Hz) 8.10-8.07 (1H, m) 8.02 (1H, d, | |
| | J = 9.0 Hz) 8.00-7.97 (1 H, m) 7.58-7.52 (2H, m) 7.44-7.38 (2H, m) 7.32 | |
| | (1 H, dd, J = 9.0; 3.0 Hz) 3.43 (3H, s) 3.07-3.02 (2H, m) 1.67-1.59 (2H, m) | |
| | 1.45-1.36 (2H, m) 1.27-1.23 (4H, m) 0.88-0.83 (3H, m) | |
| 4:4 | 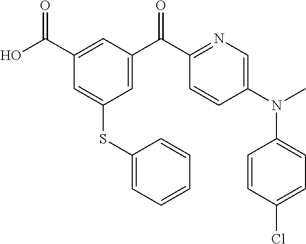 | 135 |
| | 3-{5-[(4-Chlorophenyl)(methyl)amino]picolinoyl}-5-phenylsulfanylbenzoic acid | |
| | 8.39-8.36 (1H, m) 8.12 (1H, d, J = 3.0 Hz) 8.10-8.08 (1H, m) 7.99 (1H, d, | |
| | J = 9.0 Hz) 7.95-7.92 (1 H, m) 7.60-7.55 (2H, m) 7.53-7.49 (2H, m) 7.48- | |
| | 7.43 (2H, m) 7.42-7.38 (3H, m) 7.29 (1H, dd, J = 9.0; 3.0 Hz) 3.42 (3H, s) | |
| 4:5 | 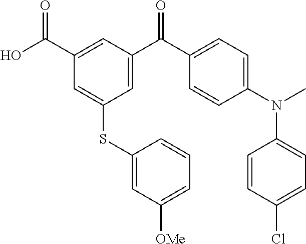 | 143 |
| | 3-{4-[(4-Chlorophenyl)(methyl)amino]benzoyl}-5-(3-methoxyphenyl-sulfanyl)benzoic acid | |
| | 13.42 (1H, s) 7.98-7.98 (1H, m) 7.95-7.94 (1H, m) 7.65-7.74 (1H, m) | |
| | 7.60-7.59 (2H, m) 7.52-7.50 (2H, m) 7.38-7.31 (3H, m) 7.05-7.03 (2H, m) | |
| | 6.98-6.96 (1H, m) 6.83-6.81 (2H, m) 3.73 (3H, s) 3.34 (3H, s) | |
| 4:6 | 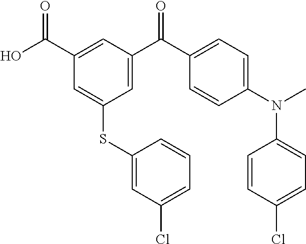 | 165 |
| | 3-{4-[(4-Chlorophenyl)(methyl)amino]benzoyl}-5-(3-chlorophenyl-sulfanyl)benzoic acid | |
| | 13.47 (1H, s) 8.04-8.03 (1H, m) 8.00-8.00 (1H, m) 7.70-7.69 (1H, m) | |
| | 7.61-7.59 (2H, m) 7.55-7.54 (1H, m) 7.52-7-50 (2H, m) 7.46-7.41 (3H, m) | |
| | 7.33-7.31 (2H, m) 6.84-6.82 (2H, m) 3.35 (3H, s) | |
| 4:7 | 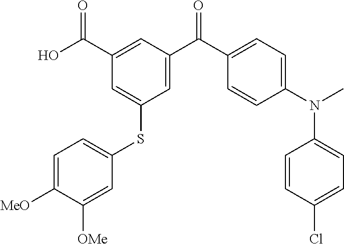 | 328 |
| | 3-{4-[(4-Chlorophenyl)(methyl)amino]benzoyl}-5-(3,4-dimethoxyphenyl-sulfanyl)benzoic acid | |
| | 13.36 (1H, m) 7.79-7.88 (1H, m) 7.83-7.82 (1H, m) 7.57-7.56 (2H, m) | |
| | 7.54-7.48 (3H, m) 7.32-7-30 (2H, m) 7.16-7.14 (2H, m) 7.06-7.05 (1H, m) | |
| | 6.82-6.80 (2H, m) 3.78 (3H, s) 3.73 (3H, s) 3.34 (3H, s) | |
| 4:8 | 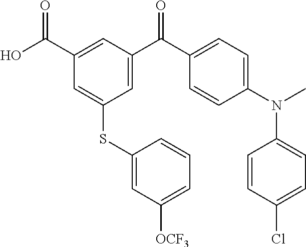 | 148 |
| | 3-{4-[(4-Chlorophenyl)(methyl)amino]benzoyl}-5-(3-(trifluoromethoxy)-phenylsulfanyl)benzoic acid | |
| | 13.48 (1H, s) 8.05-8.04 (1H, m) 8.03-8.02 (1H, m) 7.73-7.73 (1H, m) | |
| | 7.62-7.59 (2H, m) 7.58-7.54 (1H, m) 7.51-7.49 (2H, m) 7.45-7.44 (2H, m) | |
| | 7.38-7.36 (1H, m) 7.33-7.30 (2H, m) 6.84-6.81 (2H, m) 3.34 (3H, m) | |
| 4:9 | 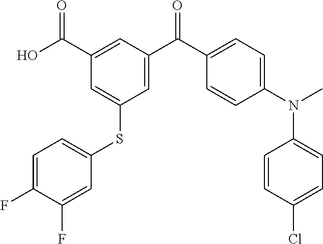 | 88 |
| | 3-{4-[(4-Chlorophenyl)(methyl)amino]benzoyl}-5-(3,4-difluorophenyl-sulfanyl)benzoic acid | |
| | 13.47 (1H, s) 8.01-8.00 (1H, m) 7.97-7.96 (1H, m) 7.71-7.67 (1H, m) | |
| | 7.66-7.65 (1H, m) 7.61-7.58 (2H, m) 7.56-7.48 (3H, m) 7.38-7.35 (1H, m) | |
| | 7.33-7.30 (2H, m) 6.84-6.81 (2H, m) 3.34 (3H, s) | |
| 4:10 | 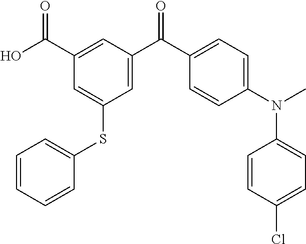 | 175 |

3-{4-[(4-Chlorophenyl)(methyl)amino]benzoyl}-5-(phenysulfanyl)benzoic acid
13.32 (1H, s) 8.43-8.40 (1H, m) 7.70-7.64 (3H, m) 7.59-7.57 (1H, m)
7.51-7.47 (2H, m) 7.39-7.23 (5H, m) 7.18-7.13 (1H, m) 7.09-7.03 (1H,
m) 6.91-6.85 (2H, m) 3.35 (3H, s)

### Example 5

Title compounds of Example 4 were tested in the biological in vitro assay described above and were found to inhibit LTC₄ synthase. Title compounds of the examples exhibit a certain IC₅₀ value, which shows that they inhibit LTC₄ synthase. IC₅₀ values for title compounds of Example 4 are depicted in the Table 4 above.

Title compounds of the Examples were tested in the biological in vitro assay described above and were found to inhibit LTC₄ synthase. Thus, when the total concentration of title compounds in the assay was 10 μM, the following %-inhibition values where obtained.

| Ex. | % inhibition |
|---|---|
| 1:1 | 100 |
| 1:2 | 100 |
| 1:3 | 100 |
| 1:13 | 99 |
| 1:21 | 99 |
| 2:1 | 99 |
| 2:2 | 98 |
| 2:3 | 96 |
| 3:1 | 97 |
| 3:2 | 98 |

Title compounds of the Examples were also tested in the biological in vitro assay described above and were found to inhibit LTC₄ synthase. The IC₅₀ values are depicted below.

| | IC₅₀ |
|---|---|
| Ex. | (nM) |
| 1:1 | 56 |
| 1:2 | 66 |
| 1:3 | 107 |
| 1:4 | 161 |
| 1:5 | 134 |
| 1:6 | 96 |
| 1:7 | 160 |
| 1:8 | 82 |
| 1:9 | 109 |
| 1:10 | 86 |
| 1:11 | 26 |
| 1:12 | 29 |
| 1:13 | 87 |
| 1:14 | 48 |
| 1:15 | 28 |
| 1:16 | 35 |
| 1:17 | 34 |
| 1:18 | 31 |
| 1:19 | 212 |
| 1:20 | 404 |
| 1:21 | 54 |
| 1:22 | 41 |
| 2:1 | 172 |
| 2:2 | 73 |
| 2:3 | 51 |
| 2:4 | 130 |
| 2:5 | 129 |
| 2:6 | 47 |
| 2:7 | 99 |
| 2:8 | 74 |
| 2:9 | 74 |
| 2:10 | 22 |
| 2:11 | 46 |
| 2:12 | 76 |
| 2:13 | 129 |
| 2:14 | 47 |
| 2:15 | 96 |
| 2:16 | 41 |
| 2:17 | 102 |
| 2:18 | 46 |
| 2:19 | 81 |
| 2:20 | 85 |
| 2:21 | 119 |
| 2:22 | 252 |
| 2:23 | 215 |
| 2:24 | 135 |
| 2:25 | 143 |
| 2:26 | 165 |
| 2:27 | 328 |
| 2:28 | 148 |
| 2:29 | 88 |
| 2:30 | 175 |
| 3:1 | 398 |
| 3:2 | 635 |

1. A compound of formula I, 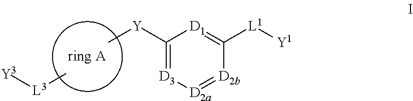
wherein
Y represents —C(O)— or —C(═N—OR²⁸)—;
either one of D₂ₐ and D_{2b} represents D₂, and the other represents —C(-L²-Y²)═;
R²⁸ represents hydrogen or C₁₋₆ alkyl optionally substituted by one or more halo atoms;
each of D₁, D₂ and D₃ respectively represent —C(R^{1a})═, —C(R^{1b})═ and —C(R^{1c})═, or, each of D₁, D₂ and D₃ may alternatively and independently represent —N═;
ring A represents: 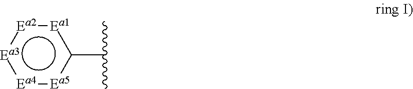
each of E^{a1}, E^{a2}, E^{a3}, E^{a4} and E^{a5} respectively represent —C(H)═, —C(R^{2b})═, —C(R^{2c})═, —C(R^{2d})═ and —C(H)═, or, each of E^{a1}, E^{a2}, E^{a3}, E^{a4} and E^{a5} may alternatively and independently represent —N═;
one of R^{2b}, R^{2c} and R^{2d} represents the requisite -L³-Y³ group, and the others independently represent hydrogen, -L^{1a}-Y^{1a} or a substituent selected from X¹; 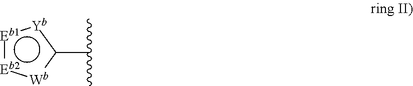
E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or 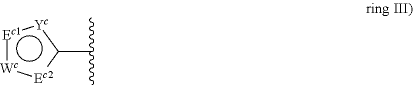
E^{c1} and E^{c2} respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c} and R^{4d} represents the requisite -L³-Y³ group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b}, R^{1c} independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂ R^{5m}, —OC(O)R⁵ⁿ, —OC(O)OR^{5p} or —OS(O)₂N(R⁶ⁱ)R⁷ⁱ;
Z^{1a} and Z^{2a} independently represent —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O, —OR^{5h} and/or R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and/or —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and/or —S(O)₂-M¹;
R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and/or —S(O)₂-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O and/or C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, R^{15a} and R^{15b} independently represent H, —CH₃ or —CH₂CH₃,
Y¹ and Y^{1a} independently represent, on each occasion when used herein, —C(O)OR^{9a} or 5-tetrazolyl;
R^{9a} represents:
(i) hydrogen; or
(ii) C₁₋₈ alkyl or a heterocycloalkyl group, both of which are optionally substituted by one or more substituents selected from G¹ and/or Z¹;
one of Y² and Y³ represents an aryl group or a heteroaryl group (both of which groups are optionally substituted by one or more substituents selected from A) and the other represents either:
(a) an aryl group or a heteroaryl group (both of which groups are optionally substituted by one or more substituents selected from A); or
(b) C₁₋₁₂ alkyl optionally substituted by one or more substituents selected from G¹ and/or Z¹;
A represents, on each occasion when used herein:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or -A¹-R^{16a};
wherein A¹ represents a single bond or a spacer group selected from —C(O)A²-, —S—, —S(O)ₘ₁A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents, on each occasion when used herein, ═O, ═S, ═NOR^{16b}, ═NS(O)₂N(R^{17f})R^{16c}, ═NCN or ═C(H)NO₂;
B represents, on each occasion when used herein:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —S(O)ₘ₁A⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂— or —S(O)₂N(R^{19e})—;
Z² represents, on each occasion when used herein, ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹—R^{20a};
wherein A¹¹ represents a single bond or a spacer group selected from —C(O)A¹²-, —S—, —S(O)ₘ₁A¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents, on each occasion when used herein, ═O, ═S, ═N0R^{20b}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L¹ and L^{1a} independently represent a single bond or —(CH₂)ₚ-Q-(CH₂)_{q}—;
Q represents —C(R^{y1})(R^{y2})—, —C(O)— or —O—;
R^{y1} and R^{y2} independently represent H, F or X⁴; or
R^{y1} and R^{y2} may be linked together to form a 3- to 6-membered ring, which ring optionally contains a heteroatom, and which ring is optionally substituted by one or more substituents selected from F, Cl, ═O and X⁵;
L² represents —S(O)ᵣ-A²¹-;
L³ represents a single bond or a spacer group selected from —(CH₂)ₚ—C(R^{y3})(R^{y4})—(CH₂)_{q}-A¹⁶-, —C(O)A¹⁷-, —S—, —S(O)—, —SC(R^{y3})(R^{y4})—, —S(O)₂A¹⁸-, —N(R^{w})A¹⁹- or —OA²⁰-, in which:
A¹⁶ represents a single bond, —O—, —N(R^{w})—, —C(O)— or —S(O)ₘ—;
A¹⁷ and A¹⁸ independently represent a single bond, —C(R^{y3})(R^{y4})—, —O— or —N(R^{w});
A¹⁹ and A²⁰ independently represent a single bond, —C(R^{y3})(R^{y4})—, —C(O)—, —C(O)C(R^{y3})(R^{y4})—, —C(O)N(R^{w})—, —C(O)O—, —S(O)₂— or —S(O)₂N(R^{w})—;
A²¹ represents a single bond or —C(R^{y3})(R^{y4})—;
p, q and r independently represent, on each occasion when used herein, 0, 1 or 2;
m represents 0, 1 or 2;
m1 represents 1 or 2;
R^{y3} and R^{y4} independently represent, on each occasion when used herein, H, F or X⁶; or
R^{y3} and R^{y4} may be linked together to form a 3- to 6-membered ring, which ring optionally contains a heteroatom, and which ring is optionally substituted by one or more substituents selected from F, Cl, ═O and X⁷;
R^{w} represents, on each occasion when used herein, H or X⁸;
X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, —CN, C₁₋₄ alkyl (optionally substituted by one or more substituents selected from fluoro, chloro and ═O), —N(R^{24c})R^{25b} and —OR^{24d})), aryl or heteroaryl (which latter two groups are optionally substituted by one or more substituents selected from halo, —CN, C₁₋₄ alkyl (optionally substituted by one or more substituents selected from fluoro, chloro and ═O), —N(R^{26a})R^{26b} and —OR^{26c});
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b} and R^{26c} are independently selected from hydrogen and C₁₋₄ alkyl, which latter group is optionally substituted by one or more substituents selected from fluoro, —OH, —OCH₃, —OCH₂CH₃ and/or ═O,
or a pharmaceutically-acceptable salt or prodrug thereof.

2. A compound as claimed in claim 1, wherein the compound of formula I represents either one of the following two formulae: 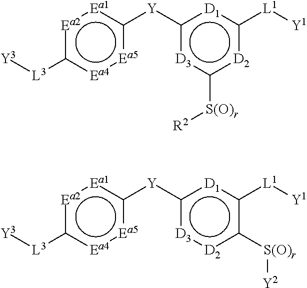
wherein:
r represents 0, 1 or 2;
Y represents —C(O)— or —C(═N—OR²⁸)—;
R²⁸ represents hydrogen or C₁₋₃ (e.g. C₁₋₂) alkyl;
each of D₁, D₂ and D₃ respectively represent —C(R^{1a})═, —C(R^{1b})═ and —C(R^{1c})═;
R^{1a}, R^{1b} and R^{1c} independently represent hydrogen, R^{5a}, halo or —CN;
each of E^{a1}, E^{a2}, E^{a4} and E^{a5} respectively represent —C(H)═, —C(R^{2b})═, —C(R^{2d})═ and —C(H)═, or, one or two of E^{a1}, E^{a2}, E^{a4} and E^{a5} may alternatively and independently represent —N═;
R^{2b} and R^{2d} independently represent hydrogen or a substituent selected from X¹;
X¹ represents a group selected from R^{5a}, halo or —CN;
R^{5a} represents C₁₋₆ alkyl optionally substituted by one or more substituents selected from ═O, halo, —CN and —N₃;
Y¹ represents —C(O)OR^{9a};
Y² represents: 5- or 6-membered heteroaryl; 9- or 10-membered bicyclic heteroaryl group; a 4- to 8-membered heterocycloalkyl group; acyclic C₁₋₆ alkyl; phenyl; or C₃₋₁₀ cycloalkyl, all of which groups are optionally substituted by one or more substituents selected from A, G¹ and/or Z¹ (as appropriate);
Y³ represents aryl optionally substituted by one or more substituents selected from A;
A represents G¹ or C₁₋₄ alkyl optionally substituted by one or more substituents selected from G¹;
G¹ represents halo or -A¹-R^{16a};
A¹ represents a single bond or —OA⁵-;
A⁵ represents a single bond;
G² represents halo or -A⁶-R^{18a};
A⁶ represents —OA¹⁰-;
A¹⁰ represents a single bond;
R^{16a} and R^{18a} independently represent hydrogen or C₁₋₄ alkyl optionally substituted by one or more fluoro atoms;
G³ represents halo;
L¹ independently represent a single bond;
L³ represents —N(R^{w})A¹⁹- or —OA²⁰-;
A¹⁹ represents a single bond;
A²⁰ represents a single bond or —C(R^{y3})(R^{y4})—;
R^{y3} and R^{y4} independently represent hydrogen;
R^{w} represents H or X⁸; and/or
X⁸ represents C₁₋₆ alkyl.

3. A compound as claimed in claim 1, wherein:
R^{1a}, R^{1b} and R^{1c} independently represent H;
ring A represents ring (I) (as depicted in claim 2);
E^{a1} and E^{a5} independently represent —C(H)═, or one or both of E^{a1} and E^{a5} may alternatively and independently represent —N═;
E^{a2}, E^{a3} and E^{a4} respectively represent —C(R^{2b})═, —C(R^{2c})═ and —C(R^{2d})═;
R^{2b} represents H;
R^{2c} represents the requisite -L³-Y³ group (as depicted in claim 2);
R^{2d} represents H;
L¹ represents a single bond;
Y¹ represents —C(O)OR^{9a};
L² represents —S(O)ᵣ—, and L³ represents —OA²⁰- or —N(R^{w})A¹⁹-; and/or
R^{w} represents C₁₋₃ alkyl or H.

4. A compound as claimed in claim 1, wherein L² represents —S(O)ᵣ— and r represents 1.

5. A compound as claimed in claim 1, wherein Y² and Y³ independently represent optionally substituted aryl, heteroaryl or C₁₋₁₂ alkyl.

6. A compound as claimed in claim 5, wherein Y² and Y³ independently represent optionally substituted phenyl or C₁₋₆ alkyl.

7. A compound of formula I as defined in claim 1, or a pharmaceutically acceptable salt thereof, for use as a pharmaceutical.

8. A pharmaceutical formulation including a compound of formula I, as defined in claim 1, or a pharmaceutically acceptable salt thereof, in admixture with a pharmaceutically acceptable adjuvant, diluent or carrier.

9. A compound, as defined in claim 1, or a pharmaceutically acceptable salt thereof, for use in the treatment of a disease in which inhibition of the synthesis of leukotriene C₄ is desired and/or required.

10. (canceled)

11. A compound as claimed in claim 9, wherein the disease is a respiratory disease, inflammation and/or has an inflammatory component.

12. A compound as claimed in claim 11 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anaemia, hypereosinofilic syndrome, or a malignancy.

13. A compound or use as claimed in claim 12, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

14. A method of treatment of a disease in which inhibition of the synthesis of leukotriene C₄ is desired and/or required, which method comprises administration of a therapeutically effective amount of a compound of formula I as defined in claim 1, or a pharmaceutically-acceptable salt thereof, to a patient suffering from, or susceptible to, such a condition.

15. A combination product comprising:
(A) a compound of formula I as defined in claim 1, or a pharmaceutically-acceptable salt thereof; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

16. A combination product as claimed in claim 15 which comprises a pharmaceutical formulation including a compound of formula I as defined in claim 1, or a pharmaceutically-acceptable salt thereof, another therapeutic agent that is useful in the treatment of a respiratory disorder and/or inflammation, and a pharmaceutically-acceptable adjuvant, diluent or carrier.

17. A combination product as claimed in claim 15 which comprises a kit of parts comprising components:
(a) a pharmaceutical formulation including a compound of formula I as defined in claim 1, or a pharmaceutically-acceptable salt thereof, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
which components (a) and (b) are each provided in a form that is suitable for administration in conjunction with the other.

18. A process for the preparation of a compound of formula I as defined in claim 1, which process comprises:
(i) for compounds of formula I in which Y represents —C(O)—, oxidation of a compound of formula II, 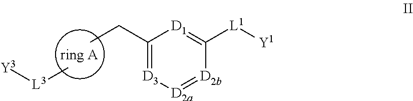
wherein ring A, D₁, D₂ₐ, D_{2b}, D₃, L¹, Y¹, L³ and Y³ are as defined in claim 1;
(ii) for compounds of formula I in which L³ represents —N(R^{w})A¹⁹- in which R^{w} represents H, reaction of a compound of formula III, 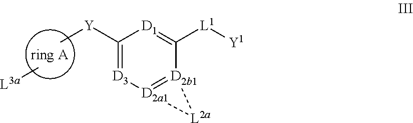
or a protected derivative thereof, wherein one of D₂ₐ₁ and D_{2b1} represents D₂ and the other represents —C(-L^{2a})= (i.e. the L^{2a} substituent is attached to either one of D₂ₐ₁ and D_{2b1}), L^{2a} represents —S(O)ᵣ—Y², L^{3a} represents —NH₂, and Y, ring A, D₁, D₂, D₃, L¹ and Y¹ are as defined in claim 1, with:
(A) when A¹⁹ represents —C(O)N(R^{w})—, in which R^{w} represents H:
(a) a compound of formula IV,
Y^{a}—N═C═O IV;
or
(b) with CO (or a reagent that is a suitable source of CO) or phosgene or triphosgene in the presence of a compound of formula V,
Y^{a}—NH₂ V
wherein, in both cases, Y^{a} represents Y² or Y³ (as appropriate/required; i.e. Y³, in this instance) as defined in claim 1;
(B) when A¹⁹ represents —S(O)₂N(R^{w})—:
(a) ClSO₃H, followed by PCl₅, and then reaction with a compound of formula V as defined above;
(b) SO₂Cl₂, followed by reaction with a compound of formula V as defined above;
(c) a compound of formula VA,
Y^{a}—N(H)SO₂Cl VA
wherein Y^{a} is as defined above;
(d) ClSO₂N═C═O, optionally in the presence BrCH₂CH₂OH, following by reaction in the presence of a compound of formula V as defined above;
(C) when A¹⁹ represents a single bond, with a compound of formula V¹,
Y^{a}-L^{a} VI
wherein L^{a} represents a suitable leaving group and Y^{a} is as defined above;
(D) when A¹⁹ represents —S(O)₂—, —C(O)—, —C(R^{y3})(R^{y4})—, —C(O)—C(R^{y3})(R^{y4})— or —C(O)O—, with a compound of formula VII,
Y^{a}-A^{19a}-L^{a} VII
wherein A^{19a} represents —S(O)₂—, —C(O)—, —C(R^{y3})(R^{y4})—, —C(O)—C(R^{y3})(R^{y4})— or —C(O)O—, and Y^{a} and L^{a} are as defined above;
(iii) for compounds of formula I in which L³ represents —N(R^{w})C(O)N(R^{w})—, in which R^{w} represents H (in both cases), reaction of a compound of formula VIII, 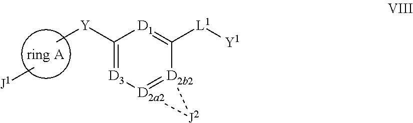
wherein one of D₂ₐ₂ and D_{2b2} represents D₂ and the other represents —C(-J²)= (i.e. the J² substituent is attached to either one of D₂ₐ₂ and D_{2b2}), one of J¹ represents —N═C═O and J² represents —S(O)ᵣ—Y², and Y, ring A, D₁, D₂ₐ, D_{2b}, D₃, L¹ and Y¹ are as defined in claim 1;
(iv) reaction of a compound of formula IX, 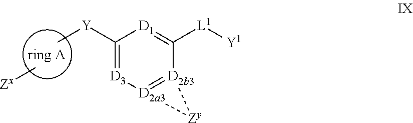
wherein one of D₂ₐ₃ and D_{2b3} represents D₂ and the other represents —C(—Z^{y})═ (i.e. the Z^{y} substituent is attached to either one of D₂ₐ₃ and D_{2b3}), and at least one of Z^{x} and Z^{y} represents a suitable leaving group and the other may also independently represent a suitable leaving group, or, Z^{y} may represent -L²-Y² and Z^{x} may represent -L³-Y³, and Y, ring A, D₁, D₂ₐ, D_{2b}, D₃, L¹, Y¹, L², Y², L³ and Y³ are as defined in claim 1, with a (or two separate) compound(s) (as appropriate/required) of formula X,
Y^{a}-L^{x}-H X
wherein L^{x} represents L² or L³ (as appropriate/required), and Y^{a} is as defined above;
(v) for compounds of formula I in which L² represents —S(O)ᵣ— in which r is 1 or 2 and, optionally, L³ represents —S(O)— or —S(O)₂— (and Y is, preferably, —C(O)— or R²⁸ is C₁₋₆ alkyl optionally substituted by one or more halo atoms), oxidation of a corresponding compound of formula I in which L² and/or L³ represents —S— (or —S(O)—, for the preparation of compounds of formula I in which there is a —S(O)₂— moiety present);
(vi) for compounds of formula I in which L² and/or L³ represents —S—, reaction of a compound of formula IX, wherein at least one of Z^{x} and Z^{y} represents a suitable leaving group and the other may also independently represent a suitable leaving group, or, Z^{y} may represent -L²-Y² and Z^{x} may represent -L³-Y³, with a (or two separate) compound(s) (as appropriate/required) of formula XI
Y^{a}—S—S—Y^{a} XI
wherein Y^{a} is as hereinbefore defined;
(vii) for compounds of formula I in which L² and/or L³ represents —S(O)₂—, reaction of a compound of formula XII, 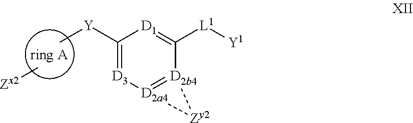
wherein one of D₂ₐ₄ and D_{2b4} represents D₂ and the other represents —C(—Z^{Y2})═ (i.e. the Z^{y2} substituent is attached to either one of D₂ₐ₄ and D_{2b4}), wherein at least one of Z^{x2} and Z^{y2} represents an appropriate alkali metal group, a —Mg-halide, a zinc-based group or a suitable leaving group, and the other may represent -L²-Y² or -L³-Y³ (as appropriate), and Y, ring A, D₁, D₂, D₃, L¹ and Y¹ are as defined in claim 1, with a (or two separate) compound(s) (as appropriate/required) of formula XIII,
Y^{a}—S(O)₂-L^{x1} XIII
wherein L^{x1} represents a suitable leaving group, and Y^{a} is as defined above;
(viii) for compounds of formula I in which L² and/or L³ represents —S(O)—, reaction of a compound of formula IX with a (or two separate) compound(s) (as appropriate/required) of formula XIV, 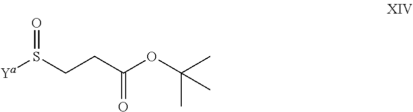
wherein Y^{a} is as defined above;
(ix) for compounds of formula I in which there is a R^{w} group present that does not represent hydrogen (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen), reaction of a corresponding compound of formula I in which such a group is present that does represent hydrogen with a compound of formula XV,
R^{wy}-L^{b} XV
wherein R^{wy} represents either R^{w} (as appropriate) as hereinbefore defined provided that it does not represent hydrogen (or R^{w} represents a R⁵ to R²⁶ group in which those groups do not represent hydrogen), and L^{b} represents a suitable leaving group;
(x) for compounds of formula I that contain only saturated alkyl groups, reduction of a corresponding compound of formula I that contains an unsaturation;
(xi) for compounds of formula I in which Y¹ and/or, if present, Y^{1a} represents —C(O)OR^{9a}, in which R^{9a} represents hydrogen (or, other carboxylic acid or ester protected derivatives), hydrolysis of a corresponding compound of formula I in which R^{9a} does not represent H;
(xii) for compounds of formula I in which Y¹ and/or, if present, Y^{1a} represents —C(O)OR^{9a}, and R^{9a} does not represent H:
(A) esterification (or the like) of a corresponding compound of formula I in which R^{9a} represents H; or
(B) trans-esterification (or the like) of a corresponding compound of formula I in which R^{9a} does not represent H (and does not represent the same value of the corresponding R^{9a} group in the compound of formula Ito be prepared),
in the presence of the appropriate alcohol of formula XVI,
R^{9za}OH XVI
in which R^{9za} represents R^{9a} provided that it does not represent H;
(xiii) for compounds of formula I in which Y¹ and/or, if present, Y^{1a} represents —C(O)OR^{9a}, in which R^{9a} is other than H, and L¹ and/or, if present, L^{1a}, are as hereinbefore defined, provided that they do not represent —(CH₂)ₚ-Q-(CH₂)_{q}— in which p represents 0 and Q represents —O—, reaction of a compound of formula XVII, 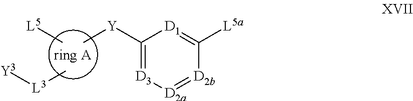
wherein at least one of L⁵ and L^{5a} represents an appropriate alkali metal group, a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L^{1a}-Y^{1a} (as appropriate), and Y, ring A, D₁, D₂ₐ, D_{2b}, D₃, L³ and Y³ are as defined in claim 1, with a compound of formula XVIII,
L⁶⁻L^{xy}-Y^{b} XVIII
wherein L^{xy} represents L¹ or L^{1a} (as appropriate; provided that it does not represent —(CH₂)ₚ-Q-(CH₂)_{q}— in which p represents 0 and Q represents —O—) and Y^{b} represents —C(O)OR^{9a}, in which R^{9a} is other than H, and L⁶ represents a suitable leaving group;
(xiv) for compounds of formula I in which L¹ and/or, if present, L^{1a} represent a single bond, and Y¹ and/or, if present, Y^{1a} represents 5-tetrazolyl, in accordance with the procedures described in international patent application WO 2006/077366;
(xv) for compounds of formula I in which L¹ and/or, if present, L^{1a} represent a single bond, and Y¹ and/or, if present, Y^{1a} represent —C(O)OR^{9a} in which R^{9a} is H, reaction of a compound of formula XVII as defined above but in which L⁵ and/or L^{5a} (as appropriate) represents either:
(I) an alkali metal; or
(II) —Mg-halide,
with carbon dioxide, followed by acidification;
(xvi) for compounds of formula I in which L¹ and/or, if present, L^{1a} represent a single bond, and Y¹ and/or, if present, Y^{1a} represent —C(O)OR^{9a}, reaction of a corresponding compound of formula XVII as defined above but in which L⁵ and/or L^{5a} (as appropriate) is a suitable leaving group with CO (or a reagent that is a suitable source of CO), in the presence of a compound of formula XIX,
R^{9a}OH XIX
wherein R^{9a} is as hereinbefore defined;
(xvii) for compounds of formula I in which Y represents —C(O)—, reaction of either a compound of formula XX or XXI, 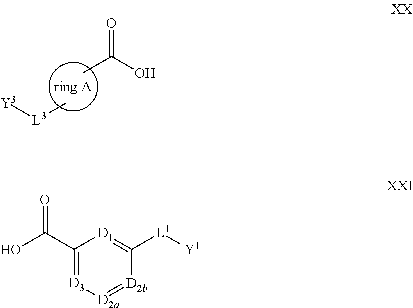
respectively with a compound of formula XXII or XXIII, 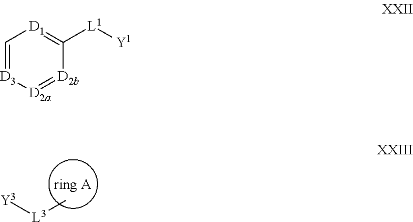
wherein (in all cases) ring A, D₁, D₂ₐ, D_{2b}, D₃, L¹, Y¹, L³ and Y³ are as defined in claim 1;
(xviii) for compounds of formula I in which Y represents —C(O)—, reaction of either a compound of formula XXIV or XXV, 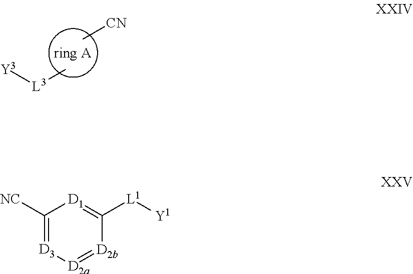
with a compound of formula XXVI or XXVII, 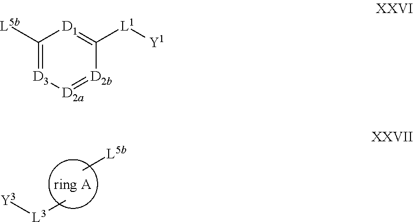
respectively, wherein L^{5b} represents L⁵ as defined above provided that it does not represent -L¹-Y¹ or -L^{1a}-Y^{1a} (i.e. it represents a suitable leaving group as defined in respect of L⁵), and (in all cases) ring A, D₁, D₂ₐ, D_{2b}, D₃, L¹, Y¹, L³ and Y³ are as defined in claim 1;
(xix) for compounds of formula I in which Y represents —C(O)—, reaction of an activated derivative of a compound of formula XX or XXI as defined above, with a compound of formula XXVII or XXVIII (as defined above), respectively;
(xx) for compounds of formula I in which Y represents —C(═N—OR²⁸)—, reaction of a corresponding compound of formula I, with a compound of formula XXVIII,
H₂N—O—R²⁸ XXVIII
wherein R²⁸ is represents hydrogen or C₁₋₆ alkyl optionally substituted by one or more halo atoms;
(xxi) for compounds of formula I in which Y represents —C(═N—OR²⁸)— and R²⁸ represents C₁₋₆ alkyl optionally substituted by one or more halo atoms, reaction of a corresponding compound of formula I, in which R²⁸ represents hydrogen, with a compound of formula XXVIIIB,
R^{28a}-L⁷ XXVIIIB
wherein R^{28a} represents R²⁸, provided that it does not represent hydrogen and L⁷ represents a suitable leaving group.

19. A process for the preparation of a pharmaceutical formulation as defined in claim 8, which process comprises bringing into association a compound of formula I, as defined in claim 1, or a pharmaceutically acceptable salt thereof with a pharmaceutically-acceptable adjuvant, diluent or carrier.

20. A process for the preparation of a combination product as defined in claim 15, which process comprises bringing into association a compound of formula I, as defined in claim 1, or a pharmaceutically acceptable salt thereof with the other therapeutic agent that is useful in the treatment of a respiratory disorder and/or inflammation, and at least one pharmaceutically-acceptable adjuvant, diluent or carrier.
